(19) United States
(12) Patent Application Publication
(10) Pub. No.: US 2010/0074857 A1
(43) Pub. Date: Mar. 25, 2010

(54) COMPOSITIONS AND METHODS TO TREAT EPITHELIAL-RELATED CONDITIONS

(76) Inventors: Pamela Lipkin, New York, NY (US); Beverly Lubit, Kinnelon, NJ (US)

Correspondence Address: GREENBERG TRAURIG, LLP, 200 PARK AVE., P.O. BOX 677, FLORHAM PARK, NJ, 07932, US

(21) Appl. No.: 12/565,335

(22) Filed: Sep. 23, 2009

Related U.S. Application Data
(60) Provisional application No. 61/099,226, filed on Sep. 23, 2008.

(51) Int. Cl. A61K 8/49; A61K 31/4164; A61K 31/496; A61K 38/02; A61Q 1/10; A61P 17/00

(52) U.S. Cl. 424/70.7; 514/399; 514/254.02; 514/2

(57) ABSTRACT

The described invention relates to the formulation and delivery of compositions to treat an epithelial-related condition selected from the group consisting of sparse hair growth, short hair growth, thin hair growth, alopecia and hair depigmentation and methods for their use. In some embodiments, the composition contains a first component and a second component wherein the first component is at least one prostaglandin analog and the second component is at least one imidazole analog, such that the at least one imidazole analog improves the efficacy of the at least one prostaglandin analog when delivered to a subject refractory to the effect of the prostaglandin analog alone.

### CROSS-REFERENCE TO RELATED APPLICATIONS

This application claims the benefit of priority to U.S. application No. 61/099,226, filed on Sep. 23, 2008, which is incorporated by reference herein in its entirety.

### FIELD OF THE INVENTION

The described invention relates to compositions comprising synthetic analogs of prostaglandin and optionally imidazole analogs.

### BACKGROUND OF THE INVENTION

### Hair

Hair, a filamentous outgrowth of protein found only on mammals, is integral to our body image and can have a profound influence on our self-esteem and self-confidence. The hair of non-human mammal species is commonly referred to as fur. In some species, hair is absent at certain stages of life.

Hair grows from hair follicles deep in the dermis and projects from the epidermis of the skin. Human skin has two types of hair: vellus hair and terminal hair. Much of human hair is short, underpigmented vellus hair rather than terminal hair. The most noticeable part of human hair is the hair on the head, which can grow longer than on most mammals and is more dense than most hair found elsewhere on the body. The term “scalp” refers to the integument of the upper part of the head, usually including the associated subcutaneous structures. The scalp is the anatomical area bordered by the face anteriorly and by the neck to the sides and posteriorly.

Vellus hair is short, fine, “peach fuzz” body hair. It is a very soft, generally pale, and short hair that grows in most places on the human body in both sexes. It usually is less than two cm long, and the follicles are not connected to sebaceous glands. It is observed most easily in women and children, as they have less terminal hair to obscure it. It also is found in pre-adolescents and in male pattern baldness.

Terminal hair is developed hair, which is generally longer, coarser, thicker and darker than the shorter and finer vellus hair. Phases of growth in terminal hair are more apparent than in vellus hair; it generally has a longer anagen phase. It has associated sebaceous glands, whereas a vellus hair may not. Under certain conditions, such as puberty, some vellus hair can become androgenic hair. Under other conditions, such as male pattern baldness, it may revert to a vellus-like state.

Each hair comprises two structures: the follicle in the skin and the shaft we see. The follicle contains several layers. At the base of the follicle is a projection called a papilla, which contains capillaries, or tiny blood vessels, that feed the cells. The living part of the hair, the area surrounding the papilla called the bulb, is the only part fed by the capillaries. The cells in the bulb divide every 23 to 72 hours, faster than any other cells in the body. The follicle is surrounded by two sheaths—an inner root sheath and an outer root sheath. These sheaths protect and mold the growing hair shaft. The inner root sheath follows the hair shaft and ends below the opening of a sebaceous (oil) gland, which produces sebum, a natural conditioner and sometimes an apocrine (scent) gland. The outer root sheath continues all the way up to the sebaceous gland. An erector pili muscle attaches below the sebaceous gland to a fibrous layer around the outer sheath. When this muscle contracts, it causes the hair to stand up.

The primary component of the hair fiber is keratin. Keratins are proteins, long chains (polymers) of amino acids. The hair shaft contains three layers of keratin. The inner layer, which is called the medulla, may not be present. The next layer is the cortex, which makes up the majority of the hair shaft. The outer layer is the cuticle, which is formed by tightly packed scales in an overlapping structure similar to roof shingles. Most hair conditioning products attempt to affect the cuticle. Pigment cells are distributed throughout the cortex and medulla giving the hair its characteristic color.

The term “eyebrow” refers to an area of coarse skin hairs above the eye that follows the shape of the brow ridges. The main function of the eyebrow is to prevent moisture, mostly salty sweat and rain, from flowing into the eye, an organ critical to sight. The typical curved shape of the eyebrow (with a slant on the side) and the direction in which eyebrow hairs are pointed, make sure that moisture has a tendency to flow sideways around the eyes, along the side of the head and along the nose. Eyebrows also prevent debris, such as dandruff and other small objects, from falling into the eyes, and provide a more sensitive sense for detecting objects being near the eye, like small insects. Eyebrows also have an important facilitative function in communication, strengthening facial expressions such as surprise, confusion, or anger.

The terms “eyelash” and “lash” are used interchangeably to refer to one of the hairs that grow at the edge of the eyelid. Eyelashes protect the eye from debris and provide a warning that an object (such as an insect or dust mite) is near the eye (which then is closed reflexively).

The inside of the nose contains small hairs called cilia. The cilia and nasal mucus clean the air drawn into the nose of the microscopic particles we inhale, including dust, pollen, and pollutants, for ultimate passage to the lungs.

### Hair Biology

There are three stages of hair growth: catagen, telogen, and anagen.

Anagen is the active growth phase of the hair during which the cells in the root of the hair are dividing rapidly. Anagen hairs are anchored deeply into the subcutaneous fat and cannot be pulled out easily. When a new hair is formed, it pushes the club hair up the follicle and eventually out. During this phase the hair grows about 1 cm every 28 days. Scalp hair stays in this active phase of growth for 2-6 years. Human subjects that have difficulty growing their hair beyond a certain length have a short active phase of growth. Human subjects that have very long hair have a long active phase of growth. The hair on the arms, legs, eyelashes, and eyebrows have a very short active growth phase of about 30-45 days, which is why they are so much shorter than scalp hair.

The anagen phase is followed by a catagen phase. The catagen phase is a transitional stage that lasts for about 2-3 weeks. About 3% of all hairs are in this phase at any time. During this time, growth stops and the outer root sheath shrinks and attaches to the root of the hair. This is the formation of what is known as a club hair.

After catagen, the hair goes into a telogen phase. Telogen is the resting phase, which accounts for 10-15% of all hairs. It lasts for about 100 days for hairs on the scalp and much longer for hairs on the eyebrow, eyelash, arm and leg. During this phase, the hair follicle is completely at rest and the club hair is completely formed. As compared with anagen hair, telogen hair is located higher in the skin and can be pulled out relatively easily. Pulling out a hair in this phase will reveal a solid, hard, dry, white material at the root. Normally, about 25-100 telogen hairs are shed each day.

In the normal scalp, approximately 80 to 90 percent of follicles are growing (anagen), about 5 to 10 percent are resting (telogen), and 1 to 3 percent are undergoing involution (catagen). Each day, up to 75 hairs in telogen are shed from the scalp and about the same number of follicles enter anagen.

The term “alopecia” is a medical term for the absence or loss of hair including eyelashes, eyebrows, and scalp hair, as a result of illness, functional disorder, or hereditary disposition. For example, the term “Alopecia adnata” refers to underdevelopment of the eyelashes. Alopecia frequently occurs in patients undergoing treatment for cancer or suffering from other diseases, such as AIDS, where cell-killing, or cytotoxic, drugs are used.

Hair loss typically is categorized as scarring or nonscarring. Scarring alopecia, also known as “cicatricial alopecia”, refers to a collection of hair loss disorders that may be diagnosed in up to 3% of hair loss patients. It occurs worldwide in otherwise healthy men and women of all ages. While there are many forms of scarring alopecia, the common theme is a potentially permanent and irreversible destruction of hair follicles and their replacement with scar tissue. Examples include bullous diseases, chemical alopecia, discoid lupus erythematosus, folliculitis (severe), lichen planopilaris, dissecting cellulitis, and tumors.

The term “nonscarring alopecia” refers to hair loss without permanent destruction of the hair follicle. Examples include anagen effluvium, androgenetic alopecia, chemical alopecia, folliculitis (mild), inherited disorders of the hair shaft, telogen effluvium, alopecia greata, and traumatic alopecia.

The term “anagen effluvium” refers to the hair loss associated with chemotherapeutic agents that cause immediate destruction and release of anagen hair.

The term “androgenic alopecia” refers to a gradual decrease of scalp hair density in adults with transformation of terminal to vellus hairs, which become lost as a result of familial increased susceptibility of hair follicles to androgen secretion following puberty. The most common form of androgenic alopecia is male pattern baldness. The most common form of androgenic alopecia in women is female pattern alopecia, a diffuse partial hair loss in the centroparietal area of the scalp, with preservation of the frontal and temporal hairlines. When it occurs in females, it is associated with other evidence of excessive androgen activity, such as hirsuitism.

The term “telogen effluvium” refers to a condition resulting from an abrupt shift of large numbers of anagen hairs to telogen hairs on the scalp, with a corresponding change in the ratio of anagen hair to telogen hair from the normal ratio of 90:10 to 70:30. This form of alopecia generally begins approximately 3 months after a major illness or other stress (e.g., surgery, parturition, rapid weight loss, nutritional deficiency, high fever, or hemorrhage) or hormonal derangement; it also has been reported after the initiation of treatment with certain medications.

The term “alopecia areata” refers to a common condition of undetermined etiology characterized by circumscribed, nonscarring, usually asymmetrical areas of baldness on the scalp, eyebrows, and bearded portion of the face. Hairy skin anywhere on the body may be affected. It is thought to be an autoimmune disease occurring on areas of the body (most commonly the scalp) where the person's immune system attacks hair follicles, thereby suppressing and arresting hair growth.

### Alopecia Related to Hormonal Changes

Telogen effluvium, a form of nonscarring alopecia characterized by diffuse hair shedding, often with an acute onset, is a reactive process caused by a metabolic or hormonal stress, or by medications. It is triggered when a physiologic stress or hormonal change causes a large number of hairs to enter telogen at one time. Physiologic stresses that may induce telogen effluvium include, but are not limited to, febrile illness, major injury, change in diet, pregnancy and delivery, and starting a new medication. Since hormonal changes in the postpartum period are a common cause of telogen effluvium, women may have a greater tendency to experience this condition. Because shedding does not occur until new anagen hairs begin to grow and the emerging hairs help to force the resting hairs out of the follicle, the interval between the inciting event in telogen effluvium and the onset of shedding corresponds to the length of the telogen phase, usually between 1 and 6 months (average of 3 months).

Telogen effluvium may be acute (hair shedding lasting less than 6 months) or chronic (hair shedding lasting longer than 6 months). Acute telogen effluvium may occur in either sex. Patients with acute telogen effluvium may complain of relatively sudden onset of hair loss.

Chronic telogen effluvium has been reported mainly in women. Generally, the onset of chronic telogen effluvium is inconspicuous and the inciting event may be difficult to identify. Patients may complain of decreased scalp hair density, and/or their hair appears thin and lifeless, due to the duration of the hair shedding.

In both forms of telogen effluvium, hair is lost diffusely from the entire scalp, and complete alopecia is not seen.

### Male Pattern Baldness

Perhaps the best known example of telogen effluvium is male pattern baldness (androgenetic alopecia; MPB; common baldness). MPB involves the conversion of androgens into dihydrotestosterone (DHT) within genetically vulnerable hair follicles. Dihydrotestosterone shortens the anagen phase and causes follicles to become miniaturized. This leads to the gradual conversion of terminal hairs into indeterminate hairs and finally to vellus hairs. Following the miniaturization of the follicles, fibrous tracts remain and patients with MPB have a reduction in the terminal-to-vellus hair ratio (normally at least 2:1).

MPB is characterized by hair receding from the lateral sides of the forehead (“receding hairline”). Receding hairlines usually are seen in males above the age of 20 years but may be seen as early as the late teens as well. An additional bald patch may develop on the top (vertex) as well. It generally is believed that MPB is controlled by a single, dominant, sex-limited, autosomal gene and may be influenced by polygenic modifying factors affecting expressivity of the trait. It can affect, to some degree, up to 50% of males between the ages of 30 and 50 years of ages.

The onset of MPB is gradual. Men present with gradual thinning in the temporal areas, producing a reshaping of the anterior part of the hairline. Baldness (the lack of all or a significant part of the hair on the head and sometimes other parts of the body) progresses to include frontal and vertex thinning. MPB in females can follow a similar course. Women usually present with diffuse thinning on the crown and less bitemporal recession than observed in men. In general, women maintain a frontal hairline.

MPB is essentially a cosmetic disorder. Other than affecting the patient psychologically, the disorder is significant only in that it allows ultraviolet light to reach the scalp, and thus increases the amount of actinic damage. It generally is believed that there exists no systemic or locally applied therapy for effective treatment of MPB, however, this has not prevented extensive study into treatment of MPB.

Punch hair transplantation or flaps provide the most effective treatment for MPB, however, this surgical treatment may require 350 to 400 grafts, transplanted in four or five operative sessions, with a minimum interval of 6 weeks between the first two sessions and four months between all subsequent sessions. Attempts to improve punch hair transplantation have included the use of scalp reductions (alopecia reductions, ARs) where a portion of a bald or balding area is excised. This results in a smaller area of alopecia which may be transplanted with fewer grafts.

Postpartum Hair Loss

It is well known that pregnancy can induce telogen effluvium in some women; however, this form of hair loss is associated more commonly with the postpartum period, occurring in about 30% to 50% of women. Postpartum hair loss is a result of the increased estrogen levels present in a woman's body during pregnancy which lead to an elongated telogen phase. This leads to fewer hairs falling out each day and a reduction of normal hair loss. After birth, hormone levels slowly balance out to their previous levels; the older hairs that were growing fall out and the fewer newer hairs that grew during pregnancy take their place.

Many women become distressed during this period. They may resort to cutting their hair short to a “wash-and-go” style thereby removing longer hair strands that are more visible when falling out. Further, many women utilize hair products, such as mousses and cremes, to add volume to their hair in attempts to provide volume to their thinning hair. Although in most women the hair loss is temporary and regrowth occurs in about 3 to 6 months, this hair loss generally is considered unavoidable.

Those suffering from hair loss often experience embarrassment and the fear being ridiculed by others because they look different. Some may take to wearing oversized eyeglasses in an attempt to hide the absence of eyelashes and/or eyebrows. Loss of nasal cilia may render some more susceptible to respiratory illnesses.

Several therapies for hair loss are designed primarily for scalp applications. These include treatment with Minoxidil (Rogaine. Minoxidil, when applied topically to the scalp, is not believed to act at a hormonal level. Rather, because of its vasodilatory effect, and because it causes hyperemia (increase in blood flow to a body part) and stimulates mitosis in epithelial cells, minoxidil directly effects the hair itself. As a topical 2% solution, minoxidil is applied twice daily for at least 2-3 months to treat MPB. The efficacy of minoxidil to grow hair is variable, and is reported to be anywhere between 6% to 40%. Side-effects of minoxidil include itching, dryness, allergic reactions, and comedones (blackheads).

In addition to topical minoxidil (Rogain®), antiandrogen agents, including the androgen-receptor blockers spironolactone, cyproterone acetate, and flutamide, and the 5α-reductase inhibitor finasteride (Propecia®, Merck & Co.), and preparations of progresterone and/or estrogen.

### Prostaglandins

Prostaglandins are a family of lipid compounds that are derived enzymatically in the body from essential fatty acids. Prostaglandins contain 20 carbon atoms, including a 5-carbon ring. They have a wide variety of effects, including, but not limited to, muscular constriction mediating inflammation, calcium movement, hormone regulation and cell growth control. Prostaglandins act on a variety of cells, including vascular smooth muscle cells (causing constriction or dilation), platelets (causing aggregation or disaggregation), and spinal neurons (causing pain).

The basic chemical structure of naturally occurring prostaglandins, as shown below, reveals that prostaglandins generally consist of a cyclopentane ring and two side chains:

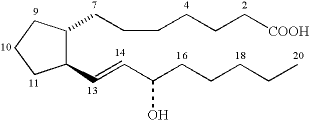

The upper side chain or “alpha chain” generally contains 7 carbon atoms. The lower side chain or “omega chain” generally contains 8 carbon atoms. The end of the alpha chain normally is a carboxylic acid moiety. The side chains may contain 1 to 3 double bonds, most frequently 2, the double bonds being situated between carbon atoms 5 and 6 on the alpha chain and between carbon atoms 13 and 14 on the omega chain. The double bond on the alpha chain generally exhibits cis-configuration, whereas the double bond on the omega chain generally exhibits trans-configuration. A substituent group on carbon 15 in the omega chain is preferred for maximal biological activity. In naturally occurring prostaglandins this substituent usually is hydroxyl.

Different classes of prostaglandins are identified by suffixes A, B, C, D, E, F or J depending on the functionalities of the five membered ring, that is the configuration and substituents of the cyclopentane ring. Prostaglandins A, B and C probably are not naturally occurring but rather are artificial prostaglandins; nevertheless, they exert considerable biologic activity.

The configuration of and functionalities attached to the cyclopentane ring is important for selectivity to different prostaglandin receptors. The various configurations include:

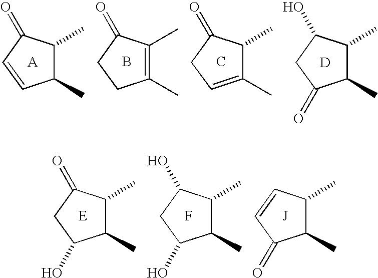

The chemical structure of known prostaglandin E1 (11α,13E,15S)-11,15-dihydroxy-9-oxoprosta-13-en-1-oic acid) (Alprostadil) is shown below wherein a hashed line represents a substituent below this paper's plane, wherein a bold wedge represents a substituent above this paper's plane:

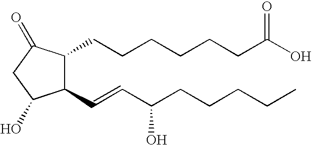

The chemical structure of known prostaglandin E2 (9-oxo-11α,15S-dihydroxyprosta-5Z,13E-dien-1-oic acid) (Dinoprostone) is shown below wherein a hashed line represents a substituent below this paper's plane; wherein a bold wedge represents a substituent above this paper's plane:

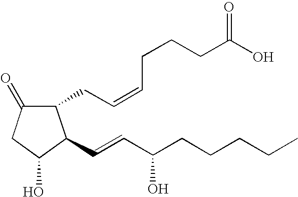

The general chemical structure of known prostaglandin D2 is shown below wherein a hashed line represents a substituent below this paper's plane; wherein a bold wedge represents a substituent above this paper's plane:

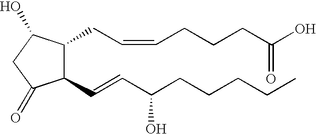

The chemical structure of prostaglandin H2 is shown below wherein a hashed line represents a substituent below this paper's plane; wherein a bold wedge represents a substituent above this paper's plane:

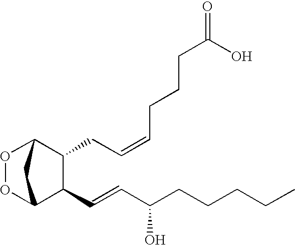

The chemical structure of prostacyclin (V)-5-((3aR,4R,5R,6aS)-5-hydroxy-4-((S,E)-3-hydroxyoct-1-enyl)hexahydro-2H-cyclopenta[b]furan-2-ylidene)pentanoic acid) (PGI₂) is shown below wherein a hashed line represents a substituent below this paper's plane; wherein a bold wedge represents a substituent above this paper's plane:

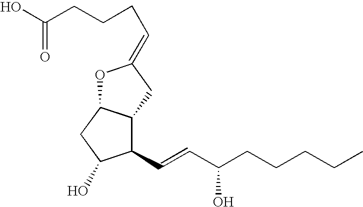

The chemical structure of prostaglandin A₂ is shown below wherein a hashed line represents a substituent below this paper's plane; and wherein a bold wedge represents a substituent above this paper's plane:

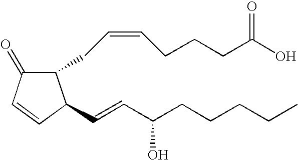

The chemical structure of known prostaglandin B₂ is shown below wherein a hashed bond represents a substituent below this paper's plane:

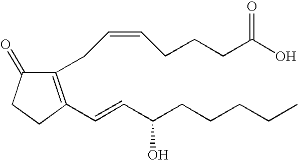

The chemical structure of known prostaglandin J₂ is shown below wherein a hashed bond represents a substituent below this paper's plane, wherein a bold wedge represents a substituent above this paper's plane, and wherein a bold wedge represents a substituent above this paper's plane:

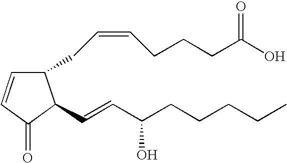

The general chemical structure of known prostaglandin F_{2α} analogs is shown below wherein a hashed line represents a substituent below this paper's plane; wherein a bold wedge represents a substituent above this paper's plane; and wherein the dashed lines represent a single or double bond which can be in the cis or trans configuration:

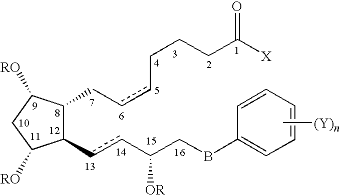

For example, latanoprost [(1R,2R,3R,5S)3,5-dihydroxy-2-[(3R)-3-hydroxy-5-phenylpentyl]cyclopentyl]-5-heptenoatel, marketed by Pfizer as Xalatan® is a prostaglandin analog in which R is H, B is —CH₂—, n is 0, X is OCH(CH₃)₂, and the dashed lines represent a double bond. See U.S. Pat. No. 6,262,105, issued to Johnstone. Although Johnstone reported the stimulating effect of this drug on eyebrow and eyelash hair growth and pigmentation, Latanoprost works poorly on eyelashes.

Another example, is bimatoprost (cyclopentane N-ethyl heptenamide-5-cis-2-(3α-hydroxy-5-phenyl-1-trans-pentenyl)-3,5-dihydroxy, [1α,2β,3α,5α], sold by Allergan, Inc. of Irvine, Calif. as Lumigan®, a 0.03% ophthalmic solution for treating glaucoma. Bimatoprost is a prostaglandin analog in which R is H, B is —CH₂—, n is 0, X is NHC₂H₅ and the dashed lines represent a double bond. See U.S. Published Application No. 2003/0147823. Bimatoprost, which also has been found effective to increase the growth of eyelashes when applied in the FDA approved manner, dissolves best for use on eyelashes but has negative side effects—e.g., redness and discoloration along the periocular skin; eye irritation; and foreign body sensation. In addition, bimtoprost has the highest incidence of hyperemia.

Another synthetic prostaglandin analog used for treatment of glaucoma is isopropyl (Z)-7-[(1-R,2-R,3-R,5-S)-3,5-dihydroxy-2-[(1E,3R)-3-hydroxy-4-[(α,α,α-trifluoro-m-tolyl)oxy]-1-butenyl]cyclopentyl]-5-heptenoate, or Travaprost (TRAVATAN® Alcon), which is available as a 0.004% ophthalmic solution. Travoprost is a prostaglandin analog in which R is H, B is O, Y is CF₃, X is OCH(CH₃)₂, n=1, and the dashed lines represent a double bond. Travaprost does not work well for eyelash growth, taking longer than other like products.

### Imidazole Analogs

Imidazole (1,3-diazacyclopenta-2,4-diene) is a five-membered aromatic heterocycle having the following structure:

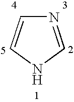

It exists in two equivalent tautomeric forms due to a hydrogen atom that may be located on either of the two nitrogen atoms.

The N-3 nitrogen atom of imidazole, which possesses a non-bonding pair of electrons, is unusually basic for an sp²-hybridized nitrogen atom. Its conjugate acid, which is called an imidazolium ion and is stabilized by resonance, has a pKₐ of approximately 7.0, as depicted below. Consequently, imidazole readily interconverts between its conjugate base and conjugate acid forms under physiological conditions, i.e. aqueous conditions near neutral pH. Furthermore, imidazole's Lewis basicity, which can be enhanced by complete or partial deprotonation of N-1, makes it an excellent ligand for many metal ions, including those that occur in biological systems.

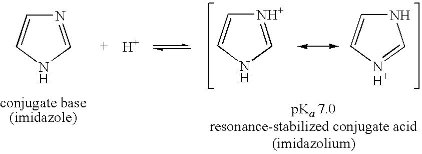

Histidine, one of the 20 endogenous amino acids that are most commonly found in proteins, contains an imidazole ring in its sidechain, which exhibits the moderate basicity and affinity for metals ions described above for imidazole itself. Due to these properties, histidine residues are essential for the normal function of many enzymes, receptors and other proteins. For example, histidine residues serve as facilitators of proton transfer in the active sites of many enzymes. Histidine residues also play several key roles in the cooperative binding and release of oxygen by hemoglobin. Decarboxylation of histidine affords histamine, an important neurotransmitter in which the imidazole moiety is essential for binding to histamine receptors.

Synthetic imidazoles are present in many fungicides, antiprotozal and antihypertensive agents. Imidazole also is part of the theophylline molecule, found in tea leaves and coffee beans, and stimulates the central nervous system. A preservative system for ophthalmic solutions comprising imidazole and a hydrogen peroxide source has been shown to be effective against fungi and bacteria (U.S. Pat. No. 6,565,894).

Examples of known imidazole analogs include, but are not limited to, histidines, the antimicrobial agents bifonazole, butoconazole, chlorimidazole, hlordantoin, croconazole, clotrimazole, democonazole, eberconazole, econazole, elubiol, enilconazole, fenticonazole, flutrimazole, isocanazole, ketoconazole, lanoconazole, lombazole, miconazole, neticonazole, NND-502, omoconazole, oxiconazole, parconazole, sertaconazole, sulconazole, tiabendazole, and tioconazole, and the thromboxane synthase inyhibitors 7-(1-imidazolyphepatanoic acid, ozagrel, and 1-benzyl imidazole. Other nitrogen-containing 5-membered aromatic heterocycles can be considered analogs of imidazole. The term “imidazole analogs” is used herein to describe imidazoles and related 5-membered aromatic heterocycles that contain at least two nitrogen atoms in the ring. Such heterocycles are exemplified, but not limited to, 1,2,4-triazole, 1,3,4-triazole, 1,2,3-triazole, tetrazole and pyrazole, as well as thiadiazoles and oxadiazoles.

Several triazoles are useful, particularly as fungicides, including albaconazole, CAS RN 214543-30-3, fluconazole, genaconzole, hydroxyitraconazole, isavuconazole, itraconazole, pramiconazole, ravuconazole, saperconazole, SYN 2869, T 8581, TAK 456, terconazole, vibunazole, voriconazole, pramiconazole, and posaconazole.

Miconazole, for example, which commonly is applied topically to the skin or to mucus membranes to treat fungal infections, such as athlete's foot and jock itch, and for vaginal yeast infections, is commercially available as a cream, lotion, powder, spray liquid, and spray powder for skin applications. Miconazole is an imidazole of the structure:

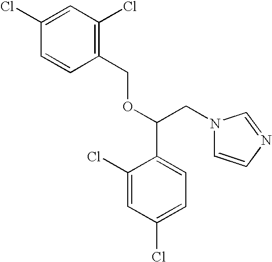

Miconazole's antifungal activity (and that of the other azole antifungals) is believed to be due to inhibition of ergosterol synthesis, specifically by inhibiting the cytochrome P450-dependent lanosterol 14α-demethylase enzyme. Ketoconazole, an imidazole anti-fungal agent having the structure:

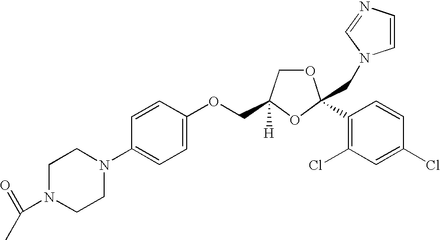

has been found to be effective in the treatment of seborrheic dermatitis. One open-label study of minoxodil 2% with ketoconazole 2% shampoo for androgenetic alopecia in men reportedly showed comparable growth in both groups, with both achieving better growth than unmedicated shampoo alone. Similar results were seen in a mouse model comparing topical ketoconazole 2% to a placebo. Ketoconazole also has been used to treat hirsutism in women, with some success. The mechanism of action is not understood.

U.S. Pat. Nos. 7,550,508, 7,514,474, 7,553,874, 7,517,912, 7,553,875, 7,541,382, and pending application Ser. Nos. 12/235,683, 12/235,736, 12/235,762, 12/235,791, 12/235,887, 12/235,966, and 12/236,024 describe prostaglandin F_{2α} analog-containing compositions and methods to treat epithelial-related conditions selected from sparse hair growth, short hair growth, thin hair growth, and hair depigmentation.

According to the described invention, additional compositions and methods comprising at least one prostaglandin analog are described. Compositions comprising at least one of the described prostaglandin analogs and at least one imidazole analog are effective at promoting appropriate growth of hair otherwise resistant to prostaglandin analogs when applied topically.

### SUMMARY OF THE INVENTION

The described invention relates to the formulation and delivery of compositions to treat an epithelial-related condition selected from the group consisting of sparse hair growth, short hair growth, thin hair growth, alopecia and hair depigmentation and methods for their use. In some embodiments, the composition contains a first component and a second component wherein the first component is at least one prostaglandin analog and the second component is at least one imidazole analog, such that the at least one imidazole analog improves the efficacy of the at least one prostaglandin analog when delivered to a subject refractory to treatment with a composition containing the prostaglandin analog alone.

The described invention relates to delivery of compositions comprising at least one prostaglandin analog, and to delivery of compositions comprising at least one prostaglandin analog and at least one imidazole analog to treat an epithelial-related condition selected from the group consisting of sparse hair growth, short hair growth, thin hair growth, alopecia and hair depigmentation, wherein the at least one imidazole analog improves the efficacy of the at least one prostaglandin analog when delivered to a subject refractory to treatment with a composition comprising the prostaglandin analog alone.

In one aspect, the described invention provides a topical composition for treating an epithelial-related condition selected from the group consisting of sparse hair growth, short hair growth, thin hair growth, alopecia, and hair depigmentation of a subject in need thereof, wherein the subject in need thereof is a subject refractory to treatment by a composition comprising a compound of Formula I alone, the composition comprising (a) a first component and a second component, (i) the first component comprising: at least one compound of Formula I or a pharmaceutically acceptable salt, hydrate, solvate, prodrug or metabolite of Formula I,

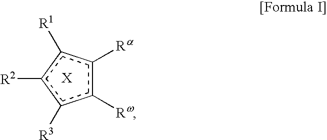

wherein ring X is selected from

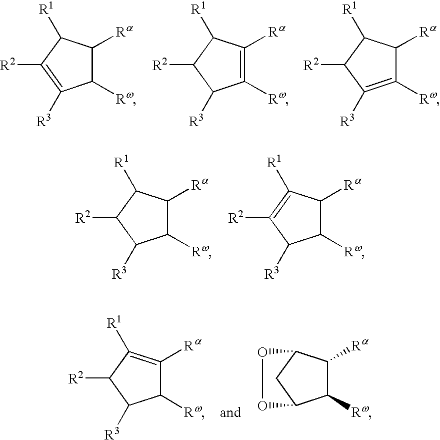

wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each; wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —PO₂(OH))ₛH where s is 1˜25 or a pharmaceutically acceptable salt thereof; or —P(O)(OH)₂ or a pharmaceutically acceptable salt thereof, wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁷ groups, wherein R^{α} is

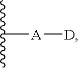

wherein A is a divalent hydrocarbon radical having from two to ten carbon atoms, which may be interrupted by one or more —O— or —S—, zero or one 1,2-phenylene, 1,3-phenylene, or 1,4-phenylene radical, and zero or one

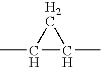

in either cis or trans configuration; the hydrocarbon radical containing zero to four C═C or C≡C bonds and zero to one C═C═C moiety; the hydrocarbon radical having no two heteroatoms adjacent and no heteroatom adjacent to a C—C multiple bond; the hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof, wherein each R⁷ is independently H, F, or straight chain or branched C₁˜C₅ alkyl, wherein M is C₃˜C₁₀ cycloalkyl containing from one to four rings, C₆˜C₁₀ aryl containing one or two rings or a 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁷ groups; wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; —NO₂; —SR¹⁰; —CH═NOR¹⁰; —C(═O)NR¹¹OR¹²; —S(O)₂ NR⁹₂; —NR⁹S(O)₂R¹³; —OS(O)²NR⁹₂; —C(O)NHS(O)₂R¹³; —S(O)₂R¹³; —SO₃H; —PO₃H₂;

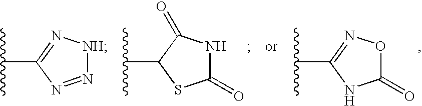

wherein R^{ω} is

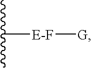

wherein E is a divalent hydrocarbon radical having from two to ten carbon atoms, which may be interrupted by one or more —O— or —S— and zero or one 1,2-phenylene, 1,3-phenylene, or 1,4-phenylene radical; the hydrocarbon radical containing zero to four C═C or C≡C bonds and zero to one C═C═C moiety; the hydrocarbon radical having no two heteroatoms adjacent and no heteroatom adjacent to a non-aromatic C—C multiple bond; the hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof, wherein F is —CH₂—; —O—; —S—; —S(O)—; —S(O₂); —C(O)—; —C(O)O—; —C(O)S—; —C(O)NR⁹—; —NR⁹—; or a covalent bond, wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁵ groups, wherein each R⁸ is independently selected from the group consisting of H; a pharmaceutically acceptable cation including but not limited to sodium, potassium, magnesium, calcium or an organic cation including but not limited to an ammonium ion; a C₁˜C₂₀ straight chain or branched acyclic hydrocarbon group, which may be interrupted by one or more —O— or —S—, the hydrocarbon group containing zero to four C═C or C≡C bonds wherein each C═C bond independently may be of E or Z configuration, the hydrocarbon group having no two heteroatoms adjacent and no heteroatom adjacent to a non-aromatic C═C or C≡C bond, the hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including but not limited to a lower alkyl ester, a lower acyloxy-alkyl ester (including but not limited to acetoxymethyl, acetoxyethyl, aminocarbonyloxymethyl, pivaloyloxymethyl or pivaloyloxyethyl ester), a lactonyl ester (including but not limited to a phthalidyl or thiophthalidyl ester), a lower alkoxyacyloxyalkyl ester (including but not limited to a methoxycarbonyloxymethyl, ethoxycarbonyloxyethyl or isopropoxycarbonyloxyethyl ester), an alkoxyalkyl ester, choline ester or acylamino alkyl ester (including but not limited to an acetamidomethyl ester); or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁵ groups, wherein each R⁹ is independently selected from the group consisting of H; a C₁˜C₂₀ straight chain or branched acyclic hydrocarbon group containing zero to four C═C or C≡C bonds wherein each C═C bond independently may be of E or Z configuration; a C₁˜C₂₀ straight chain or branched acyl group containing zero to four C═C or C≡C bonds wherein each C═C bond independently may be of E or Z configuration; —(CH₂)_{q}OH, —(CH₂)_{q}OR¹⁴, —(CH₂)_{q}OC(O)R₁₄, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁵ groups; —CH₂CH(OH)CH₂OH; —CH(CH₂OH)₂; —CH₂CH(CH₂OH)₂; lower acyloxy-alkyl (including but not limited to acetoxymethyl, acetoxyethyl, aminocarbonyloxymethyl, pivaloyloxymethyl or pivaloyloxyethyl), lactonyl (including but not limited to a phthalidyl or thiophthalidyl), lower alkoxyacyloxyalkyl (including but not limited to a methoxycarbonyloxymethyl, ethoxycarbonyloxyethyl or isopropoxycarbonyloxyethyl), or acylamino alkyl (including but not limited to acetamidomethyl); or -J-K; or —NR⁹₂ may be a cycloamido radical (including but not limited to 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R9 groups which may be alike or different, or 1-piperazinyl substituted at the 4-position by R9, and the like), wherein each R¹⁰ is independently H; a C₁˜C₂₀ straight chain or branched acyclic hydrocarbon group containing zero to four C═C or C≡C bonds wherein each C═C bond independently may be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁷ groups; or -L-M, wherein L is a covalent bond or a C₁˜C₁₀ straight chain or branched alkyl, wherein each R¹¹ is independently H or —C(O)R¹⁶, wherein each R¹² is independently R¹⁶ or —C(O)R¹⁶, wherein each R¹³ is independently a C₁˜C₂₀ straight chain or branched acyclic hydrocarbon group, which may be interrupted by one or more —O— or —S—, the hydrocarbon group containing zero to four C═C or C≡C bonds wherein each C═C bond independently may be of E or Z configuration, the hydrocarbon group having no two heteroatoms adjacent and no heteroatom adjacent to a non-aromatic C═C or C≡C bond, the hydrocarbon group being substituted with zero to four R¹⁷ groups; —(CH²)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁷ groups; -L-M; or L-O-M (“O” being oxygen), wherein each R¹⁴ is independently straight chain or branched C₁˜C₆ alkyl or —CH₂OCH₃, wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; —CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R₅; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂ N(R¹⁶)₂; —NR¹⁶S(O)₂R¹³; —C(O)NHS(O)₂R¹³; —S(O)₂R¹³; —SO₃H; —PO₃H₂;

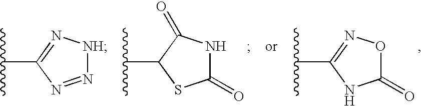

wherein each R¹⁶ is independently H or straight chain or branched C₁˜C₆ alkyl, phenyl or —CH₂OCH₃, wherein each R¹⁷ is independently straight chain or branched C₁˜C₆ alkyl; —C(O)OR¹⁶; phenyl; F; Cl; Br; I; —CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂ N(R¹⁶)₂; —NR¹⁶S(O)₂R¹³, with the proviso that, if R¹⁷ is —NR¹⁶S(O)₂R₁₃, R¹, R², R³, R^{α} and R^{ω} are selected such that the molecular weight of the compound of Formula III does not exceed about 2000 atomic mass units; (ii) the second component comprising at least one compound of Formula IV or a hydrate, solvate, salt, zwitterion, N-oxide, prodrug, metabolite, or tautomer thereof:

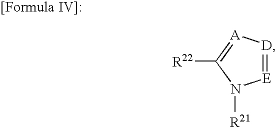

wherein each A is independently N or —NR²⁰; wherein each D is independently CR²³; wherein each E is independently N or CR²⁴; wherein R²³ is H; wherein R²⁴ is H; or wherein R²³ and R²⁴ together are —CH═CH—CH═CH—; wherein R²⁰ is H and the compound of Formula IV is an imidazolium or triazolium salt with a pharmaceutically acceptable counter anion; or wherein R²⁰ is a moiety that is readily cleaved in vivo, exemplified by, but not limited to, —CH₂OC(O)CH₃, and the compound of Formula IV is a prodrug and an imidazolium or triazolium salt with a pharmaceutically acceptable counter anion; wherein R²² is H; —CH₃; or

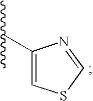

wherein R²¹ is H; —(CR²⁵R²⁶)ₘ—CR²⁷R²⁸-Q-R²⁹; wherein each m is independently 0 or 1; wherein R²⁵ is H; —CH₃; or —C≡N; wherein R²⁶ is H; wherein R²⁷ is -T-U—V; wherein R²⁸ is H; —OH; or

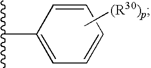

wherein each Q is independently a covalent bond or —S—; wherein R²⁹ is

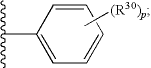

or straight chain or branched C₃˜C₁₀ alkyl optionally substituted with —CO₂H; wherein each p is independently an integer from 0 to 3 inclusive; wherein each T is independently a covalent bond; —CH₂CH₂—; —OCH₂—; —CH₂O—; —SCH₂—; —CH₂S—; —OCH₂CH₂O—; —CH(CH₃); —CH₂—; or —CF₂; wherein each U is independently a covalent bond;

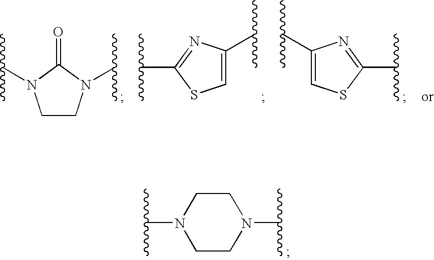

wherein each V is independently H; —S(O)₂CH₃; —C(O)NH₂; —CH₂C—CH; —CH═CH₂;

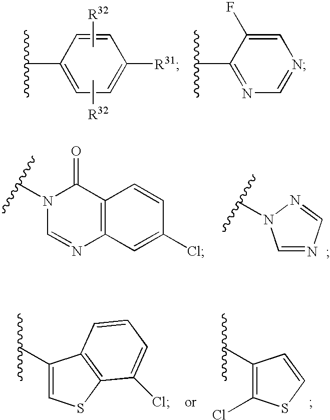

wherein each R³⁰ is independently F, Cl, —CH═CH—CO₂H;

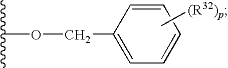

or straight chain or branched C₁˜C₁₀ alkoxy; wherein each R³¹ is independently H; F; Cl; Br; I;

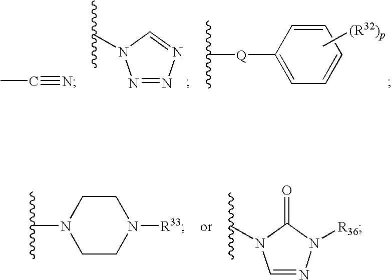

wherein each R³² is independently H; F; Cl; or —OCF₃; wherein each R³³ is independently straight chain or branched C₁˜C₆ alkyl; —C(O)R³⁴; —C(O)OR³⁴;

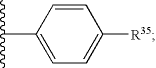

wherein each R³⁴ is independently straight chain or branched C₁˜C₆ alkyl; wherein each R³⁵ is independently H;

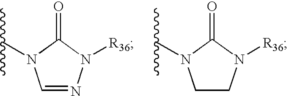

wherein each R³⁶ is independently straight chain or branched C₁˜C₆ alkyl optionally substituted with —OH; or

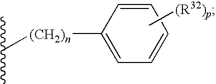

wherein each n is independently 0 or 1; wherein R²⁶ and R²⁸ together with the two carbon atoms to which they attach may be C═C; wherein R²⁷ and R²⁸ together may be

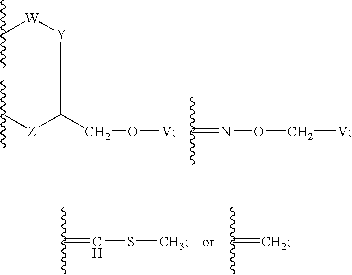

wherein W is —CH₂— and Y is —CH₂—; W is —O— and Y is —CH₂—; or W is —CH₂— and Y is —O—; wherein Z is —CH₂— or —O—; wherein R²⁷ together with -Q-R²⁹ may be

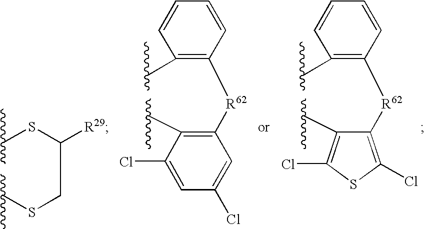

wherein each R⁶² is independently —CH₂—CH₂— or —CH═CH—; and (b) a carrier; wherein the composition stimulates hair growth on an epithelial surface to which the composition has been applied. According to one embodiment, the imidazole analog of Formula IV is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a hydrate, solvate, salt, zwitterion, prodrug, metabolite or tautomer thereof. According to another embodiment, the imidazole analog is miconazole or ketoconazole or a hydrate, solvate, salt, zwitterion, prodrug, metabolite or tautomer thereof. According to another embodiment, the at least one compound of Formula IV is diastereomerically pure. According to another embodiment, the at least one compound of Formula IV is a mixture of diastereomers in any ratio. According to another embodiment, the at least one compound of Formula IV is enantiomerically pure. According to another embodiment, the at least one compound of Formula IV is a mixture of enantiomers in any ratio, including a racemate. According to another embodiment, the at least one compound of Formula IV is diastereomerically and enantiomerically pure. According to another embodiment, the at least one compound of Formula IV is a mixture of diastereomers and enantiomers in any ratio. According to another embodiment, the at least one compound of Formula IV has one or more hydrogen atoms replaced by deuterium. According to another embodiment, the at least one compound of Formula I is diastereomerically pure. According to another embodiment, the at least one compound of Formula I is a mixture of diastereomers in any ratio. According to another embodiment, the at least one compound of Formula I is enantiomerically pure. According to another embodiment, the at least one compound of Formula I is a mixture of enantiomers in any ratio, including a racemate. According to another embodiment, the at least one compound of Formula I is diastereomerically and enantiomerically pure. According to another embodiment, the at least one compound of Formula I is a mixture of diastereomers and enantiomers in any ratio. According to another embodiment, the at least one compound of Formula I has one or more hydrogen atoms replaced by deuterium. According to another embodiment, the at least one compound of Formula I is at least one compound selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin 7 analog. According to another embodiment, the prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA_{I} N-cyclopropylmethylamide, 17-phenyl-18,19,20-trinor PGA₂ N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, and 6-(3-chlorophenyl)-17,18,19,20-tetranor PGA₁ N-(1,3-dihydroxypropan-2-yl))amide; (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof. According to another embodiment, the prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁ N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB₁ N-(1,3-dihydroxypropan-2-yl))amide; (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyehept-5-enoate, (2)-N-ethyl-7-(2-((R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof. According to another embodiment, the prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, and 6-(3-chlorophenyl)-17,18,19,20-tetranor PGC₁ N-(1,3-dihydroxypropan-2-yl))amide; (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD_{I} N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, and 6-(3-chlorophenyl)-17,18,19,20-tetranor PGD_{I} N-(1,3-dihydroxypropan-2-yl))amide; (Z)-isopropyl 7-((1R,2R,5S)-5-hydroxy-2-((R)-3-hydroxy-5-phenylpentyl)-3-oxocyclopentyl)hept-5-enoate, (Z)-isopropyl 7-((1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (Z)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-(1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof. According to another embodiment, the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, and 6-(3-chlorophenyl)-17,18,19,20-tetranor PGE₁ N-(1,3-dihydroxypropan-2-yl))amide; (Z)-isopropyl 7-((1R,2R,3R)-3-hydroxy-2-((R)-3-hydroxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof. According to another embodiment, the prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF₂, N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof. According to another embodiment, the prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ_{I} N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂ N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, and 6-(3-chlorophenyl)-17,18,19,20-tetranor PGJ₁ N-(1,3-dihydroxypropan-2-yl))amide; (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z)-N-ethyl-7-(1S,5R)-5-((R,E)-3-hydroxy-4-(3-(trifluoromethyl)phenoxy)but-1-enyl)-4-oxocyclopent-2-enyl)hept-5-enamide, (2) —N-ethyl-7-((1S,5R)-5-((S,E)-3-hydroxy-5-phenylpent-1-enyl)-4-oxocyclopent-2-enyl)hept-5-enamide, (Z)-7-((1S,5R)-5-((R,E)-3-hydroxy-4-(3-(trifluoromethyl)phenoxy)but-1-enyl)-4-oxocyclopent-2-enyl)hept-5-enoic acid, (Z)-7-((1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-(1S,5R)-5-((R,E)-4-(3-chlorophenoxy)-3-hydroxybut-1-enyl)-4-oxocyclopent-2-enyl)hept-5-enoie acid, (Z)-isopropyl 7-((1S,5R)-5-((R,E)-4-(3-chlorophenoxy)-3-hydroxybut-1-enyl)-4-oxocyclopent-2-enyl)hept-5-enoate, (Z)-7-((1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof. According to another embodiment, the topical composition is formulated as a mascara. According to another embodiment, the topical composition is an ophthalmic composition. According to another embodiment, the composition restores pigmentation to depigmented hair. According to another embodiment, the epithelial-related surface onto which the composition is applied topically is an eyelid, at least one eyelash, a face, an eyebrow, a scalp, and above a lip. According to another embodiment, the composition further comprises at least one additional active ingredient selected from the group consisting of a protective agent, an emollient, an astringent, an irritant, a keratolytic, a sun screening agent, a sun tanning agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent, or a combination thereof. According to another embodiment, the alopecia is a telogen effluvium type. According to some such embodiments, the telogen effluvium type is male pattern baldness. According to some such embodiments, the telogen effluvium type is postpartum hair loss.

In another aspect, the described invention provides a method for treating an epithelial-related condition selected from the group consisting of sparse hair growth, short hair growth, thin hair growth, alopecia and hair depigmentation, of a subject in need thereof, wherein the subject in need thereof is a subject refractory to treatment by a composition comprising a compound of Formula I alone, the method comprising the steps: (a) preparing a composition comprising a first component, a second component; and a carrier; (i) the first component comprising at least one compound of Formula I or a pharmaceutically acceptable salt, hydrate, solvate, prodrug or metabolite thereof,

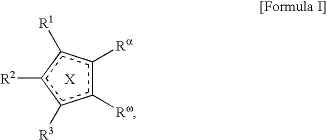

wherein ring X is selected from

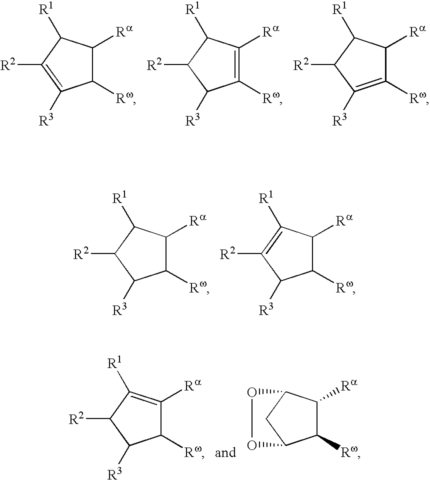

wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each; wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; PO₂(OH))ₛH where s is 1-25 or a pharmaceutically acceptable salt thereof or —P(O)(OH)₂ or a pharmaceutically acceptable salt thereof; wherein each R⁵ is independently H, saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁷ groups; wherein R^{α} is

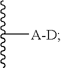

wherein A is a divalent hydrocarbon radical having from two to ten carbon atoms, which may be interrupted by one or more —O— or —S—, zero or one 1,2-phenylene, 1,3-phenylene, or 1,4-phenylene radical, and zero or one

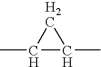

in either cis or trans configuration; the hydrocarbon radical containing zero to four C═C or C≡C bonds and zero to one C═C═C moiety; the hydrocarbon radical having no two heteroatoms adjacent and no heteroatom adjacent to a C—C multiple bond; the hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof; wherein each R⁷ is independently H, F, or straight chain or branched C₁˜C₅ alkyl; wherein M is C₃˜C₁₀ cycloalkyl containing from one to four rings, C₆˜C₁₀ aryl containing one or two rings or a 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁷ groups; wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; ; —NO₂; —SR¹⁰; —CH═NOR¹⁰; —C(═O)NR¹¹OR¹²; —S(O)₂ NR⁹₂; —NR⁹S(O)₂R¹³; —OS(O)²NR⁹₂; —C(O)NHS(O)₂R¹³; —S(O)₂R¹³; —SO₃H; —PO₃H₂;

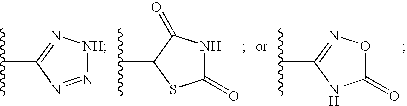

wherein R^{ω} is

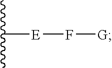

wherein E is a divalent hydrocarbon radical having from two to ten carbon atoms, which may be interrupted by one or more —O— or —S— and zero or one 1,2-phenylene, 1,3-phenylene, or 1,4-phenylene radical; the hydrocarbon radical containing zero to four C═C or C≡C bonds and zero to one C═C═C moiety; the hydrocarbon radical having no two heteroatoms adjacent and no heteroatom adjacent to a non-aromatic C—C multiple bond; the hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof; wherein F is —CH₂—; —O—; —S—; —S(O)—; —S(O₂)—; —C(O)—; —C(O)O—; —C(O)S—; —C(O)NR⁹—; —NR⁹—; or a covalent bond; wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁵ groups; wherein each R⁸ is independently selected from the group consisting of: H; a pharmaceutically acceptable cation including but not limited to sodium, potassium, magnesium, calcium or an organic cation including but not limited to an ammonium ion; a C₁˜C₂₀ straight chain or branched acyclic hydrocarbon group, which may be interrupted by one or more —O— or —S—, the hydrocarbon group containing zero to four C═C or C≡C bonds wherein each C═C bond independently may be of E or Z configuration, the hydrocarbon group having no two heteroatoms adjacent and no heteroatom adjacent to a non-aromatic C═C or C≡C bond, the hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including but not limited to a lower alkyl ester, a lower acyloxy-alkyl ester (including but not limited to acetoxymethyl, acetoxyethyl, aminocarbonyloxymethyl, pivaloyloxymethyl or pivaloyloxyethyl ester), a lactonyl ester (including but not limited to a phthalidyl or thiophthalidyl ester), a lower alkoxyacyloxyalkyl ester (including but not limited to a methoxycarbonyloxymethyl, ethoxycarbonyloxyethyl or isopropoxycarbonyloxyethyl ester), an alkoxyalkyl ester, choline ester or acylamino alkyl ester (including but not limited to an acetamidomethyl ester); or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁵ groups; wherein each R⁹ is independently selected from the group consisting of: H; a C₁˜C₂₀ straight chain or branched acyclic hydrocarbon group containing zero to four C═C or C≡C bonds wherein each C═C bond independently may be of E or Z configuration; a C₁˜C₂₀ straight chain or branched acyl group containing zero to four C═C or C≡C bonds wherein each C═C bond independently may be of E or Z configuration; —(CH₂)_{q}OH, —(CH₂)_{q}OR¹⁴, —(CH₂)_{q}OC(O)R₁₄, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁵ groups; —CH₂CH(OH)CH₂OH; —CH(CH₂OH)₂; —CH₂CH(CH₂OH)₂; lower acyloxy-alkyl (including but not limited to acetoxymethyl, acetoxyethyl, aminocarbonyloxymethyl, pivaloyloxymethyl or pivaloyloxyethyl), lactonyl (including but not limited to a phthalidyl or thiophthalidyl), lower alkoxyacyloxyalkyl (including but not limited to a methoxycarbonyloxymethyl, ethoxycarbonyloxyethyl or isopropoxycarbonyloxyethyl), or acylamino alkyl (including but not limited to acetamidomethyl); or -J-K; or —NR⁹₂ may be a cycloamido radical (including but not limited to 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which may be alike or different, or 1-piperazinyl substituted at the 4-position by R⁹, and the like); wherein each R¹⁰ is independently H; a C¹˜C²⁰ straight chain or branched acyclic hydrocarbon group containing zero to four C═C or C≡C bonds wherein each C═C bond independently may be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁷ groups; or -L-M, wherein L is a covalent bond or a C₁˜C₁₀ straight chain or branched alkyl; wherein each R¹¹ is independently H or —C(O)R¹⁶; wherein each R¹² is independently R¹⁶ or —C(O)R¹⁶; wherein each R¹³ is independently a C₁˜C₂₀ straight chain or branched acyclic hydrocarbon group, which may be interrupted by one or more —O— or —S—, the hydrocarbon group containing zero to four C═C or C≡C bonds wherein each C═C bond independently may be of E or Z configuration, the hydrocarbon group having no two heteroatoms adjacent and no heteroatom adjacent to a non-aromatic C═C or C≡C bond, the hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁷ groups; -L-M; or -L-O-M (“O” being oxygen); wherein each R¹⁴ is independently straight chain or branched C₁˜C₆ alkyl or CH₂OCH₃; wherein each R¹⁵ is independently straight chain or branched C₁˜C₆ alkyl; straight chain or branched fluoro-substituted C₁˜C₆ alkyl; straight chain or branched fluoro-substituted C₁˜Cₒ alkoxy; straight chain or branched C1˜C4 alkyl substituted with one, two or three hydroxyl groups; —C(O)OR¹⁶; phenyl; phenyl substituted with one to three R¹⁷; F; Cl; Br; I; —CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂ N(R¹⁶)₂; —NR¹⁶S(O)₂R¹³; —C(O)NHS(O)₂R¹³; —S(O)₂R¹³; —SO₃H; —PO₃H₂;

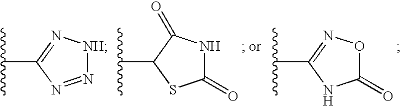

wherein each R¹⁶ is independently H or straight chain or branched C₁˜C₆ alkyl, phenyl or —CH₂OCH₃; wherein each R¹⁷ is independently straight chain or branched C₁˜C₆ alkyl; —C(O)OR¹⁶; phenyl; F; Cl; Br; I; —CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂ N(R¹⁶)₂; —NR¹⁶S(O)₂R¹³, with the proviso that, if R¹⁷ is —NR¹⁶S(O)₂R₁₃, R¹, R², R³, Rα and Rω are selected such that the molecular weight of the compound of Formula III does not exceed about 2000 atomic mass units; (ii) the second component comprising at least one compound of Formula IV or a hydrate, solvate, salt, zwitterion, N-oxide, prodrug, metabolite, or tautomer thereof:

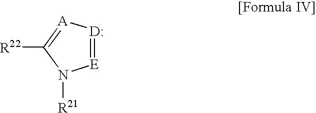

wherein each A is independently N or —NR²⁰; wherein each D is independently CR²³; wherein each E is independently N or CR²⁴; wherein R²³ is H; wherein R²⁴ is H; or wherein R²³ and R²⁴ together are —CH═CH—CH═CH—; wherein R²⁰ is H and the compound of Formula IV is an imidazolium or triazolium salt with a pharmaceutically acceptable counter anion; or wherein R²⁰ is a moiety that is readily cleaved in vivo, exemplified by, but not limited to, —CH₂OC(O)CH₃, and the compound of Formula IV is a prodrug and an imidazolium or triazolium salt with a pharmaceutically acceptable counter anion; wherein R²² is H; —CH₃; or

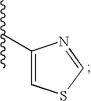

wherein R²¹ is H; —(CR²⁵R²⁶)ₘ—CR²⁷R²⁸-Q-R²⁹; wherein each m is independently 0 or 1; wherein R²⁵ is H; —CH₃; or —C≡N; wherein R²⁶ is H; wherein R²⁷ is -T-U—V; wherein R²⁸ is H; —OH; or

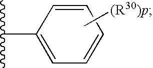

wherein each Q is independently a covalent bond or —S—; wherein R²⁹ is

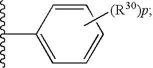

or straight chain or branched C₃˜C₁₀ alkyl optionally substituted with —CO₂H; wherein each p is independently an integer from 0 to 3 inclusive; wherein each T is independently a covalent bond; —CH₂CH₂—; —OCH₂—; —CH₂O—; —SCH₂—; —CH₂S—; —OCH₂CH₂O—; —CH(CH₃)—; —CH₂—; or —CF₂—; wherein each U is independently a covalent bond;

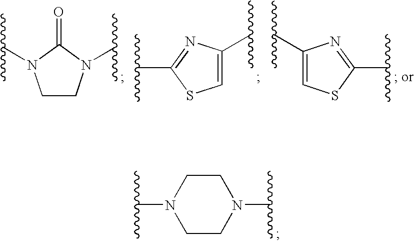

wherein each V is independently H; —S(O)₂CH₃; —C(O)NH₂; —CH₂C≡CH; —CH═CH₂;

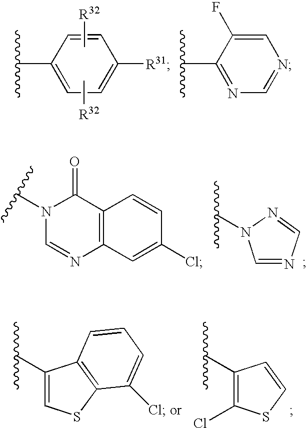

wherein each R³⁰ is independently F, Cl, —CH═CH—CO₂H;

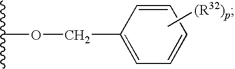

or straight chain or branched C₁˜C₁₀ alkoxy; wherein each R³¹ is independently H; F; Cl; Br; I;

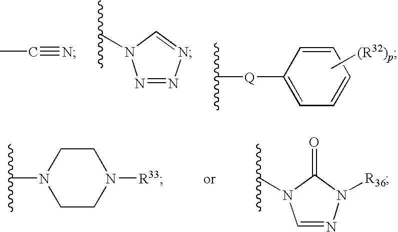

wherein each R³² is independently H; F; Cl; or —OCF₃; wherein each R³³ is independently straight chain or branched C₁˜C₆ alkyl; —C(O)R³⁴; —C(O)OR³⁴;

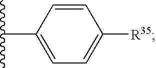

wherein each R³⁴ is independently straight chain or branched C₁˜C₆ alkyl; wherein each R³⁵ is independently H;

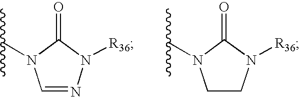

wherein each R³⁶ is independently straight chain or branched C₁˜C₆ alkyl optionally substituted with —OH; or

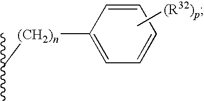

wherein each n is independently 0 or 1; wherein R²⁶ and R²⁸ together with the two carbon atoms to which they attach may be C═C; wherein R²⁷ and R²⁸ together may be

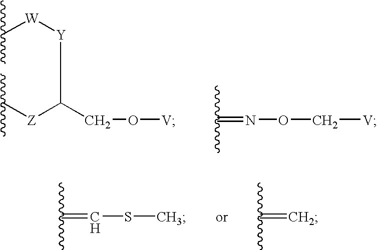

wherein W is —CH₂— and Y is —CH₂—; W is —O— and Y is —CH₂—; or W is —CH₂— and Y is —O—; wherein Z is —CH₂— or —O—; wherein R²⁷ together with -Q-R²⁹ may be

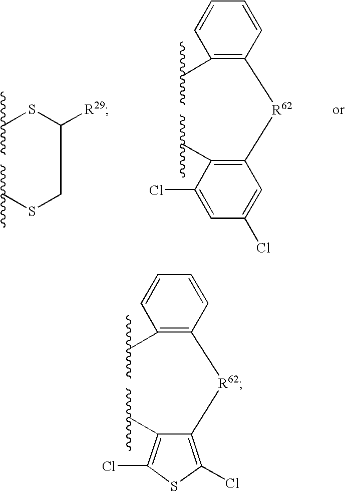

wherein each R⁶² is independently —CH₂CH₂— or —CH═CH—; wherein each chiral center independently may possess any relative or absolute stereoconfiguration or be any mixture thereof, unless specified otherwise herein; wherein each olefinic C═C bond that is capable of E/Z isomerism independently may possess either the E or Z stereoconfiguration or be any mixture thereof, unless specified otherwise herein; (b) topically applying a cosmetically effective amount of the composition onto an epithelial surface of the subject, and (c) stimulating hair growth on an epithelial surface to which the composition has been applied. According to one embodiment, the imidazole analog of Formula IV is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a hydrate, solvate, salt, zwitterion, prodrug, metabolite or tautomer thereof; According to another embodiment, the imidazole analog is miconazole or ketoconazole or a hydrate, solvate, salt, zwitterion, prodrug, metabolite or tautomer thereof. According to another embodiment, the at least one compound of Formula IV is diastereomerically pure. According to another embodiment, the at least one compound of Formula IV is a mixture of diastereomers in any ratio. According to another embodiment, the at least one compound of Formula IV is enantiomerically pure. According to another embodiment, the at least one compound of Formula IV is a mixture of enantiomers in any ratio, including a racemate. According to another embodiment, the at least one compound of Formula IV is diastereomerically and enantiomerically pure. According to another embodiment, the at least one compound of Formula IV is a mixture of diastereomers and enantiomers in any ratio. According to another embodiment, the at least one compound of Formula IV has one or more hydrogen atoms replaced by deuterium. According to another embodiment, the at least one compound of Formula I is diastereomerically pure. According to another embodiment, the at least one compound of Formula I is a mixture of diastereomers in any ratio. According to another embodiment, the at least one compound of Formula I is enantiomerically pure. According to another embodiment, the at least one compound of Formula I is a the at least one compound of Formula I is diastereomerically and enantiomerically pure. According to another embodiment, the at least one compound of Formula I is a mixture of diastereomers and enantiomers in any ratio. According to another embodiment, the at least one compound of Formula I has one or more hydrogen atoms replaced by deuterium. According to another embodiment, the at least one compound of Formula I is at least one compound selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog. According to another embodiment, the prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl)amide, 17-phenyl-18,19,20-trinor PGA₂ N-cyclopropylamide, 17-phenyl-18,19,20-trinor PGA_{I} N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA; N-cyclopropylmethylamide, and 6-(3-chlorophenyl)-17,18,19,20-tetranor PGA_{I} N-(1,3-dihydroxypropan-2-yl))amide (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyehept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid, (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof. According to another embodiment, the prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁ N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB_{I} N-(1,3-dihydroxypropan-2-yl))amide; (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z) —N-ethyl-7-(2-((R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide, or a pharmaceutically acceptable salt, hydrate, solvate, prodrug or metabolite thereof. According to another embodiment, the prostaglandin C analog is selected from the group consisting of 16-phenoxy-17,18,19,20-tetranor PGC₂ N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, and 6-(3-chlorophenyl)-17,18,19,20-tetranor PGC₁ N-(1,3-dihydroxypropan-2-yl))amide; (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, and 6-(3-chlorophenyl)-17,18,19,20-tetranor PGD₁ N-(1,3-dihydroxypropan-2-yl))amide; (Z)-isopropyl 7-((1R,2R,5S)-5-hydroxy-2-((R)-3-hydroxy-5-phenylpentyl)-3-oxocyclopentyl)hept-5-enoate, (Z)-isopropyl 7-(1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (2)-7-(1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof. According to another embodiment, the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, and 6-(3-chlorophenyl)-17,18,19,20-tetranor PGE₁ N-(1,3-dihydroxypropan-2-yl))amide; (Z)-isopropyl 7-(1R,2R,3R)-3-hydroxy-2-((R)-3-hydroxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-(1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof. According to another embodiment, the prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF₂ᵢ, N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 1643-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof. According to another embodiment, the prostaglandin J analog is selected from the group consisting of 16-phenoxy-17,18,19,20-tetranor PGD₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGD₂ N-cyclopropylamide, 17-phenyl-18,19,20-trinor PGJ₁ N-cyclopropylmethylamide, 17-phenyl-18,19,20-trinor PGJ₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGJ₁ N-cyclopropylmethylamide, and 6-(3-chlorophenyl)-17,18,19,20-tetranor PGJ₁ N-(1,3-dihydroxypropan-2-yl))amide; (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z)-N-ethyl-7-((1S,5R)-5-((R,E)-3-hydroxy-4-(3-(trifluoromethyl)phenoxy)but-1-enyl)-4-oxocyclopent-2-enyl)hept-5-enamide, (Z)-N-ethyl-7-((1S,5R)-5-((S,E)-3-hydroxy-5-phenylpent-1-enyl)-4-oxocyclopent-2-enyl)hept-5-enamide, (Z)-7-((1S,5R)-5-((R,E)-3-hydroxy-4-(3-(trifluoromethyl)phenoxy)but-1-enyl)-4-oxocyclopent-2-enyl)hept-5-enoic acid, (Z)-7-((1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-((1S,5R)-5-((R,E)-4-(3-chlorophenoxy)-3-hydroxybut-1-enyl)-4-oxocyclopent-2-enyl)hept-5-enoic acid, (Z)-isopropyl 7-((1S,5R)-5-((R,E)-4-(3-chlorophenoxy)-3-hydroxybut-1-enyl)-4-oxocyclopent-2-enyl)hept-5-enoate, (Z)-7-((1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof. According to another embodiment, the topical composition is formulated as a mascara. According to another embodiment, the topical composition is an ophthalmic composition. According to another embodiment, the composition restores pigmentation to depigmented hair. According to another embodiment, the epithelial-related surface onto which the composition is applied topically is an eyelid, at least one eyelash, a face, an eyebrow, a scalp, and above a lip. According to another embodiment, the composition further comprises at least one additional active ingredient selected from the group consisting of a protective agent, an emollient, an astringent, an irritant, a keratolytic, a sun screening agent, a sun tanning agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent, or a combination thereof. According to another embodiment, the alopecia is a form of nonscarring alopecia. According to another embodiment, the nonscarring alopecia is of a telogen effluvium type. According to some such embodiments, the telogen effluvium type of nonscarring alopecia is male pattern baldness. According to some such embodiments, the telogen effluvium type of nonscarring alopecia is postpartum hair loss.

According to another aspect, the described invention provides a method for treating an epithelial-related condition selected from the group consisting of sparse hair growth, short hair growth, thin hair growth, alopecia and hair depigmentation, the method comprising the steps: (a) formulating a composition comprising (i) at least one prostaglandin analog according to Formula II or a pharmaceutically acceptable salt, hydrate, solvate, prodrug, or metabolite thereof,

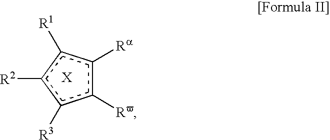

wherein ring X is selected from the group consisting of

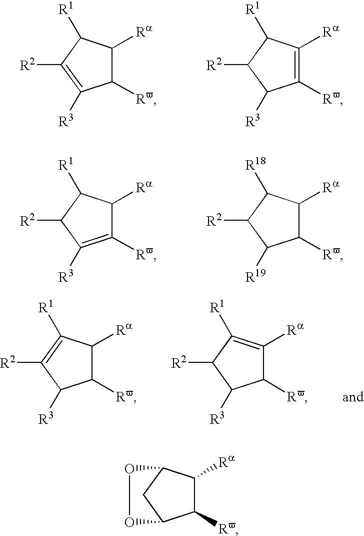

wherein R¹, R², R³, R¹⁸ and R¹⁹ are independently H, —OR⁴, ═O, or —OC(O)R⁵, where the carbon atoms to which R¹, R², R³, R¹⁸ and R¹⁹ attach bear the appropriate number of additional H atoms so as to have exactly 4 bonds each, with the proviso that, when R¹⁸ is —OR⁴ or —OC(O)R⁵ and is cis to R^{α} and trans to R^{ω} with respect to the plane of the cyclopentane ring, then R¹⁹ is H or ═O; wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —PO₂(OH))ₛH wherein s is 1˜25 or a pharmaceutically acceptable salt thereof; or —P(O)(OH)₂ or a pharmaceutically acceptable salt thereof; wherein each R⁵ is independently saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁷ groups; wherein R^{α} is

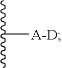

wherein A is a divalent hydrocarbon radical having from two to ten carbon atoms, which may be interrupted by one or more —O— or —S—, zero or one 1,2-phenylene, 1,3-phenylene, or 1,4-phenylene radical, and zero or one

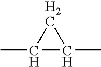

in either cis or trans configuration; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof; wherein each R⁷ is independently H, F, or straight chain or branched C₁˜C₅ alkyl; wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁷ groups; wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; —NO₂; —SR¹⁰; —CH═NOR¹⁰; —C(O)NR¹¹OR¹²; —S(O)₂ NR⁹₂; —NR⁹S(O)₂R¹³; —OS(O)₂ NR⁹₂; —C(O)NHS(O)₂R¹³; —S(O)₂R¹³; —SO₃H; —PO₃H₂;

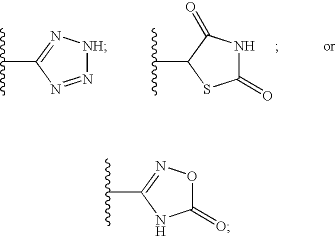

wherein R^{ω} is

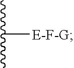

wherein E is a divalent hydrocarbon radical having from two to ten carbon atoms, which may be interrupted by one or more —O— or —S— and zero or one 1,2-phenylene, 1,3-phenylene, or 1,4-phenylene radical; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═S, —O(CO)R⁵ or R⁷ groups; wherein each olefinic moiety may independently be E or Z and each allenic moiety or chiral center may independently possess any relative or absolute stereoconfiguration or any mixture thereof; wherein F is —CH₂; —O—; —S—; —S(O)—; —S(O₂); —C(O)—; —C(O)O—; —C(O)S—; —C(O)NR⁹—; —NR⁹—; or a covalent bond; wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁵ groups; wherein each R⁸ is independently selected from the group consisting of H; a pharmaceutically acceptable cation including but not limited to sodium, potassium, magnesium, calcium or an organic cation including but not limited to an ammonium ion; a C₁˜C₂₀ straight chain or branched acyclic hydrocarbon group, which may be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including but not limited to a lower alkyl ester, a lower acyloxy-alkyl ester (including but not limited to acetoxymethyl, acetoxyethyl, aminocarbonyloxymethyl, pivaloyloxymethyl or pivaloyloxyethyl ester), a lactonyl ester (including but not limited to a phthalidyl or thiophthalidyl ester), a lower alkoxyacyloxyalkyl ester (including but not limited to a methoxycarbonyloxymethyl, ethoxycarbonyloxyethyl or isopropoxycarbonyloxyethyl ester), an alkoxyalkyl ester, choline ester or acylamino alkyl ester (including but not limited to an acetamidomethyl ester); or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁵ groups; wherein each R⁹ is independently selected from the group consisting of: H; a C₁˜C₂₀ straight chain or branched acyclic hydrocarbon group containing zero to four C═C or C≡C bonds wherein each C═C bond independently may be of E or Z configuration; a C₁˜C₂₀ straight chain or branched acyl group containing zero to four C═C or C≡C bonds wherein each C═C bond independently may be of E or Z configuration; —(CH₂)_{q}OH, —(CH₂)_{q}OR¹⁴, —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl (including but not limited to acetoxymethyl, acetoxyethyl, aminocarbonyloxymethyl, pivaloyloxymethyl or pivaloyloxyethyl), lactonyl (including but not limited to a phthalidyl or thiophthalidyl), lower alkoxyacyloxyalkyl (including but not limited to a methoxycarbonyloxymethyl, ethoxycarbonyloxyethyl or isopropoxycarbonyloxyethyl), or acylamino alkyl (including but not limited to acetamidomethyl); or -J-K; or —NR⁹₂ may be a cycloamido radical (including but not limited to 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which may be alike or different, or 1-piperazinyl substituted at the 4-position by R⁹, and the like); wherein each R¹⁰ is independently H; a C₁˜C₂₀ straight chain or branched acyclic hydrocarbon group containing zero to four C═C or C≡C bonds wherein each C═C bond independently may be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl; wherein each R¹¹ is independently H or —C(O)R¹⁶; wherein each R¹² is independently R¹⁶ or —C(O)R¹⁶; wherein each R¹³ is independently a C₁˜C₂₀ straight chain or branched acyclic hydrocarbon group, which may be interrupted by one or more —O— or —S—, said hydrocarbon group containing zero to four C═C or C≡C bonds wherein each C═C bond independently may be of E or Z configuration, said hydrocarbon group having no two heteroatoms adjacent and no heteroatom adjacent to a non-aromatic C═C or bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or L-O-M (“O” being oxygen); wherein each R¹⁴ is independently straight chain or branched C₁—-C₆ alkyl or —CH₂OCH₃; wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; —CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂N(R¹⁶)₂; —NR¹⁶S(O)₂R¹³; —C(O)NHS(O)₂R¹³; —S(O)₂R¹³; —SO₃H; —PO₃H₂;

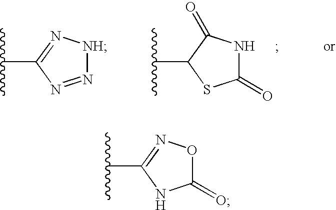

wherein each R¹⁶ is independently H or straight chain or branched C₁˜C₆ alkyl, phenyl or —CH₂OCH₃; wherein each R¹⁷ is independently straight chain or branched C₁˜C₆ alkyl; —C(O)OR¹⁶; phenyl; F; Cl; Br; I; —CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂N(R¹⁶)₂; —NR¹⁶S(O)₂R¹³, with the proviso that, if R¹⁷ is —NR¹⁶S(O)₂R¹³, R¹, R², R³, R¹⁸, R¹⁹, R^{α} and R^{ω} are selected such that the molecular weight of the compound of Formula II does not exceed about 2000 atomic mass units; and wherein not more than four of R⁷ are other than H or F and not more than four of R⁷ are F; and (ii) a carrier; and (b) topically applying a cosmetically effective amount of the composition onto an epithelial surface of a subject, including a human, in need thereof. According to one embodiment, the at least one compound of Formula II is diastereomerically pure. According to another embodiment, the at least one compound of Formula II is a mixture of diastereomers in any ratio. According to another embodiment, the at least one compound of Formula II is enantiomerically pure. According to another embodiment, the at least one compound of Formula II is a mixture of enantiomers in any ratio, including a racemate. According to another embodiment, the at least one compound of Formula II is diastereomerically and enantiomerically pure. According to another embodiment, the at least one compound of Formula II is a mixture of diastereomers and enantiomers in any ratio. According to another embodiment, the at least one compound of Formula II has one or more hydrogen atoms replaced by deuterium. According to another embodiment, the at least one compound of Formula II is at least one compound selected from the group consisting of a prostaglandin A analog, a prostaglandin B analog, a prostaglandin C analog, a prostaglandin D analog, a prostaglandin E analog, a prostaglandin I analog and a prostaglandin J analog. According to another embodiment, the prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, and 6-(3-chlorophenyl)-17,18,19,20-tetranor PGA₁ N-(1,3-dihydroxypropan-2-yl))amide; (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid, (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof. According to another embodiment, the prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB_{I} N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB_{I} N-(1,3-dihydroxypropan-2-yl))amide; (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)-N-ethyl-7-(2-((R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, [fluprostenol isopropyl ester is same as travoprost], (Z)-7-(2-((R,E)-3-hydroxy-4-(3-(trifluoromethyl)phenoxy)but-1-enyl)-5-oxocyclopent-1-enyl)-N-methylhept-5-enamide, [9-keto fluprostenol isopropyl ester is same as PGE analog of Travoprost], (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, and (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof. According to another embodiment, the prostaglandin C analog is selected from the group consisting of 16-phenoxy-17,18,19,20-tetranor PGC₂ N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-tritnor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, and 6-(3-chlorophenyl)-17,18,19,20-tetranor PGC₁ N-(1,3-dihydroxypropan-2-yl))amide; (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyehept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 1643-chlorophenyl)-17,18,19,20-tetranor PGD₁ N-cyclopropylmethylamide, and 6-(3-chlorophenyl)-17,18,19,20-tetranor PGD_{I} N-(1,3-dihydroxypropan-2-yl))amide; (Z)-isopropyl 7-((1R,2R,5S)-5-hydroxy-2-((R)-3-hydroxy-5-phenylpentyl)-3-oxocyclopentyl)hept-5-enoate, (Z)-isopropyl 7-((1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (Z)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof. According to another embodiment, the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, and 6-(3-chlorophenyl)-17,18,19,20-tetranor PGE₁ N-(1,3-dihydroxypropan-2-yl))amide; (Z)-isopropyl 7-((1R,2R,3R)-3-hydroxy-2-((R)-3-hydroxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof. According to another embodiment, the prostaglandin J analog is selected from the group consisting of 16-phenoxy-17,18,19,20-tetranor PGJ₂ N-cyclopropylamide, 16-phenoxy-17,18,19,20-tetranor PGJ_{I} N-cyclopropylmethylamide, 16-phenoxy-17,18,19,20-PGJ_{I} N-(1,3-dihydroxypropan-2-yl))amide; 17-phenyl-18,19,20-trinor PGJ₂ N-cyclopropylamide, 17-phenyl-18,19,20-trinor PGJ_{I} N-cyclopropylmethylamide, 17-phenyl-18,19,20-trinor PGJ₂ N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, and 6-(3-chlorophenyl)-17,18,19,20-tetranor PGJ₁ N-(1,3-dihydroxypropan-2-yl))amide; (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z)-N-ethyl-7-((1S,5R)-5-((R,E)-3-hydroxy-4-(3-(trifluoromethyl)phenoxy)but-1-enyl)-4-oxocyclopent-2-enyl)hept-5-enamide, (Z)-N-ethyl-7-(1S,5R)-5-((S,E)-3-hydroxy-5-phenylpent-1-enyl)-4-oxocyclopent-2-enyl)hept-5-enamide, (Z)-7-(1S,5R)-5-((R,E)-3-hydroxy-4-(3-(trifluoromethyl)phenoxy)but-1-enyl)-4-oxocyclopent-2-enyl)hept-5-enoic acid, (Z)-7-(1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-(1S,5R)-5-((R,E)-4-(3-chlorophenoxy)-3-hydroxybut-1-enyl)-4-oxocyclopent-2-enyl)hept-5-enoic acid, (Z)-isopropyl 7-((1S,5R)-5-((R,E)-4-(3-chlorophenoxy)-3-hydroxybut-1-enyl)-4-oxocyclopent-2-enyl)hept-5-enoate, (Z)-7-((1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof. According to another embodiment, the topical composition is formulated as a mascara. According to another embodiment, the topical composition is an ophthalmic composition. According to another embodiment, the composition restores pigmentation to depigmented hair. According to another embodiment, the epithelial-related surface onto which the composition is applied topically is an eyelid, at least one eyelash, a face, an eyebrow, a scalp, and above a lip. According to another embodiment, the composition further comprises at least one additional active ingredient selected from the group consisting of a protective agent, an emollient, an astringent, an irritant, a keratolytic, a sun screening agent, a sun tanning agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent, or a combination thereof. According to another embodiment, the alopecia is a form of nonscarring alopecia. According to another embodiment, the nonscarring alopecia is of a telogen effluvium type. According to some such embodiments, the telogen effluvium type of nonscarring alopecia is male pattern baldness. According to some such embodiments, the telogen effluvium type of nonscarring alopecia is postpartum hair loss.

### DETAILED DESCRIPTION OF THE INVENTION

The described invention relates to delivery of compositions comprising at least one prostaglandin analog and to delivery of compositions comprising at least one prostaglandin analog and at least one imidazole analog to treat an epithelial-related condition selected from the group consisting of sparse hair growth, short hair growth, thin hair growth, alopecia and hair depigmentation, wherein the at least one imidazole analog improves the efficacy of the at least one prostaglandin analog when delivered to a subject refractory to treatment with a composition comprising the prostaglandin analog alone.

### GLOSSARY

The phrase “additional active ingredient” as used herein refers to an agent, other than a compound of the inventive composition, that exerts a pharmacological, dermatological or any other beneficial activity. It is to be understood that “other beneficial activity” may be one that is only perceived as such by the subject using the inventive compositions.

The term “anesthetic agents” as used herein refers to agents that resulting in a reduction or loss of sensation. Non-limiting examples of anesthetic drugs that are suitable for use in the context of the described invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

The term “acne” as used herein refers to an inflammatory disease of the sebaceous glands, characterized by comedones and pimples. The term “anti-acne” as used herein refers to agents that alleviate the symptoms of acne. Examples of anti-acne agents include, without limitation, keratolytics, such as salicylic acid, sulfur, glycolic, pyruvic acid, resorcinol, and N-acetylcysteine; and retinoids such as retinoic acid and its derivatives (e.g., cis and trans, esters).

The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin ; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate ; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

The term “anti-fungal agent” as used herein means any of a group of chemical substances having the capacity to inhibit the growth of or to destroy fungi. Anti-fungal agents include, but are not limited to, Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, PyrroInitrin, Siccanin, Tubercidin, Viridin, Butenafine, Naftifine, Terbinafine, Bifonazole, Butoconazole, Chlordantoin, Chlormidazole, Croconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconazole, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

The term “anti-dandruff agents” as used herein refers to agents that reduce, eliminate or prevent a scurf from forming on skin, especially of the scalp, that comes off in small white or grayish scales. Exemplary anti-dandruff ingredients usable in context of the described invention include, without limitation, zinc pyrithione, shale oil and derivatives thereof such as sulfonated shale oil, selenium sulfide, sulfur; salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazole nitrate and any possible stereo isomers and derivatives thereof such as anthralin, piroctone olamine (Octopirox), selenium sulfide, and ciclopiroxolamine, and mixtures thereof.

The term “antihistamine agent” as used herein refers to any of various compounds that counteract histamine in the body and that are used for treating allergic reactions (such as hay fever) and cold symptoms. Non-limiting examples of antihistamines usable in context of the described invention include chlorpheniramine, brompheniramine, dexchlorpheniramine, tripolidine, clemastine, diphenhydramine, promethazine, piperazines, piperidines, astemizole, loratadine and terfenadine.

The term “anti-irritant” as used herein refers to an agent that prevents or reduces soreness, roughness, or inflammation of a bodily part.

The term “anti-protozoal agent” as used herein means any of a group of chemical substances having the capacity to inhibit the growth of or to destroy protozoans used chiefly in the treatment of protozoal diseases. Examples of antiprotozoal agents, without limitation, include pyrimethamine (Daraprim®), sulfadiazine, and Leucovorin.

The term “antipruritic agents” as used herein refers to those substances that reduce, eliminate or prevent itching. Suitable antipruritic agents include, without limitation, pharmaceutically acceptable salts of methdilazine and trimeprazine.

The term “anti-oxidant agent” as used herein refers to a substance that inhibits oxidation or reactions promoted by oxygen or peroxides. Non-limiting examples of anti-oxidants that are usable in the context of the described invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

The term “anti-skin atrophy actives” refers to substances effective in replenishing or rejuvenating the epidermal layer by promoting or maintaining the natural process of desquamation. Non-limiting examples of antiwrinkle and antiskin atrophy actives which can be used in context of the described invention include retinoic acid, its prodrugs and its derivatives (e.g., cis and trans) and analogues; salicylic acid and derivatives thereof, sulfur-containing D and L amino acids and their derivatives and salts, particularly the N-acetyl derivatives, an example of which is N-acetyl L-cysteine; thiols, e.g. ethane thiol; alpha-hydroxy acids, e.g. glycolic acid, and lactic acid; phytic acid, lipoic acid; lysophosphatidic acid, and skin peel agents (e.g., phenol and the like).

The term “anti-viral agent” as used herein means any of a group of chemical substances having the capacity to inhibit the replication of or to destroy viruses used chiefly in the treatment of viral diseases. Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Zidovudine, Acemannan, Acetylleucine, Amantadine, Amidinomycin, Delavirdine, Foscamet, Indinavir, Interferons (e.g., IFN-alpha), Kethoxal, Lysozyme, Methisazone, Moroxydine, Nevirapine, Podophyllotoxin, Ribavirin, Rimantadine, Ritortavir2, Saquinavir, Stailimycin, Statolon, Tromantadine, Zidovudine (AZT) and Xenazoic Acid.

The term “astringents” are generally protein precipitants that have such a low cell penetrability that the action essentially is limited to the cell surface and interstitial spaces. Astringents are locally applied. The astringent action is accompanied by contraction and wrinkling of the tissue and by blanching. Astringents are used therapeutically to arrest hemorrhage by coagulating the blood, to promote healing, to toughen the skin or to decrease sweating. The principal components of astringents are salts of aluminum, zinc, manganese, iron or bismuth.

The term “carrier” as used herein describes a material that does not cause significant irritation to an organism and does not abrogate the biological activity and properties of the compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both.

The term “caustic agents” as used herein refers to substances capable of destroying or eating away epithelial tissue by chemical action. Caustic agents can be used to remove dead skin cells. For example, beta-hydroxy acids, naturally derived acids with a strong kerolytic effect, are useful for problem skin, acne or peeling.

The terms “chelating agent”, “chelant” or “chelator” refers to a chemical that forms soluble complex molecules with certain metal ions thereby inactivating the ions.

The term “chemotherapetic agent” as used herein refers to chemicals useful in the treatment or control of a disease. Non-limiting examples of chemotherapeutic agents usable in context of the described invention include fluorouracil, capecitabine, mercaptopurine, gemcitabine HCl, pemetrexed disodium, floxuridine, erlotinib, sorafenib, decitabine, angrelide HCl, vorinostat, octreotide acetate, bexarotene, isotretinoin, megestrol acetate, fluoxymesterone, leuprolide acetate, temozolomide, busulfan, melphalan HCl, carmustine, lomustine, mesna, ifosfamide, mesnex, dacarbazine, oxaliplatin, irinotecan HCl, topotecan HCl, teniposide, etoposide, etoposide phosphate, mitoxantrone, filgrastim, pegfilgrastim, bevacizumab, sargramostim, leukine, panitumumab, mitomycin, dactinomycin, daunorubicin HCl, epirubicin HCl, doxorubicin HCl, doxorubicin, idarubicin HCl, dexraoxane, paclitaxel, docetaxel, ixabepilone, vincristine sulfate, vinblastine sulfate, vinorelbine tartrate, daunorubicin, idarubicin, amrubicin, pirarubicin, epirubicin, vinblastine, vincristine, mitomycin C, 5-FU, actinomycin D, colchicine, gemcitabine cyclosporin, verapamil, valspodor, probenecid, MK571, GF 120918, LY335979, biricodar, terfenadine, quinidine, pervilleine A and XR9576.

The term “color” as used herein refers to the quality of an object or substance with respect to light reflected or absorbed by the object or substance. The three characteristics of color are hue, intensity, and value. “Hue” refers to a gradation, tint, or variety of a color. “Intensity”, “chroma”, and “saturation” are used interchangeably to refer to the strength or sharpness of a color. A color is full in intensity only when pure and unmixed. “Value” refers to a degree of lightness or darkness in a color.

The term “compatible” as used herein means that the components of a composition are capable of being combined with each other in a manner such that there is no interaction that would substantially reduce the efficacy of the composition under ordinary use conditions.

The term “condition” as used herein includes a variety of conditions related to skin or mucosal membranes. This term is meant to include disorders or diseases, the promotion of healthy epithelium; dry skin; and inflammation caused by any underlying mechanism or disorder.

The term “cosmetic composition’ as used herein refers to a composition that is intended to be rubbed, poured, sprinkled, or sprayed on, introduced into, or otherwise applied to a subject or any part thereof for cleansing, beautifying, promoting attractiveness, or altering the appearance, or an article intended for use as a component of any such article, except that such term does not include soap.

The phrase “cosmetically acceptable carrier” as used herein refers to a substantially non-toxic carrier, conventionally useable for the topical administration of cosmetics, with which compounds will remain stable and bioavailable.

The term “colorant” as used herein refers to substance, dye, pigment, ink or paint that colors or modifies the hue of something. Colorants include pigments or dyes or a combination thereof as the cosmetic benefit requires.

The term “demulcents” as used herein refers to protective agents employed primarily to alleviate irritation, particularly mucous membranes or abraded tissues. They often are applied to the surface in a viscid, sticky preparation that covers the area readily and may be medicated. A number of chemical substances possess demulcent properties. These substances include, but are not limited to, the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

The term “derivative” as used herein means a compound that may be produced from another compound of similar structure in one or more steps. A “derivative” or “derivatives” of a peptide or a compound retains at least a degree of the desired function of the peptide or compound. Accordingly, an alternate term for “derivative” may be “functional derivative.”

The terms “drying agent” or “dessicant” as used herein refers to a substance that has an affinity for water such that it will extract the water from other materials.

The term “emollients” as used herein generally refers to bland, fatty or oleaginous materials which can be applied locally, particularly to the skin. Emollients increase the tissue moisture content, thereby rendering the skin softer and more pliable. Increased moisture content in the skin can be achieved by preventing water loss with an occlusive water-immiscible barrier, by increasing the water-holding capacity in the skin with humectants, or by altering the desquamation of the outermost skin layer, the stratum corneum. Useful emollients include, but are not limited to, lanolin, spermaceti, mineral oil, paraffin, petrolatum, white ointment, white petroleum, yellow ointment. Also included are vegetable oils, waxes, cetyl alcohol, glycerin, hydrophilic petrolatum, isopropyl myristate, myristyl alcohol, and oleyl alcohol.

The term “emulsifiers” as used herein are agents that promote the formation and stabilization of an emulsion.

The term “emulsion” as used herein refers to a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil.

The term “epithelia” or “epithelial” or “epithelial tissues” as used herein is meant to include skin and mucosal membranes.

The phrase “new excipient” as used herein means any inactive ingredient that is intentionally added to the composition of the described invention and is not intended to exert therapeutic effects at the intended dosage, although it may act to improve product delivery. A new excipient is not fully qualified by existing safety data with respect to the currently proposed level of exposure, duration of exposure or route of administration. Additional characteristics of new excipients can be found in the Guidance for Industry Nonclinical Studies for the Safety Evaluation of Pharmaceutical Excipients issued by the US Food and Drug Administration Center for Drug Evaluation and Research, in May, 2005, herein incorporated by reference.

The term “fusion protein” as used herein refers to a protein created through the joining of two or more genes which originally coded for separate proteins. Translation of the fusion gene results in a single polypeptide with functional properties derived from each of the original proteins.

The term “hormone” as used herein refers to natural substances produced by organs of the body that travel by blood to trigger activity in other locations or their synthetic analogs. Suitable hormones for use in the context of the described invention include, but are not limited to, calciferol (Vitamin D3) and its products.

The term “hypopigmenting agents” as used herein refers to substances capable of depigmenting the skin. Suitable hypopigmenting agents include, but are not limited to, hydroquinones, mequinol, and various protease inhibitors including serine protease inhibitors, active soy and retinoic acid.

The term “irritant” as used herein refers to a material that acts locally on the skin to induce, based on irritant concentration, hyperemia (meaning an excess of blood in an area or body part, usually indicated by red, flushed color or heat in the area), inflammation, and desiccation. Irritant agents include, but are not limited to, alcohol, aromatic ammonia spirits, benzoin tincture, camphor capsicum, and coal tar extracts.

The term “keratolytics” (desquamating agents) as used herein refers to an agent that acts to remove outer layers of the stratum corneum. They are particularly useful in hyperkeratotic areas. The keratolytics include, but are not limited to, benzoyl peroxide, fluorouracil, resorcinol, salicylic acid, tretinoin, and the like.

The term “male pattern baldness” (“MPB”; “androgenetic alopecia”) as used herein refers to a progressive, diffuse loss of scalp hair.

The term “moisturizing agent” as used herein refers to a substance that adds or restores moisture to the skin.

The term “non-steroidal anti-inflammatory agents” as used herein refers to a large group of agents that are aspirin-like in their action, including, but not limited to, ibuprofen (Advil®), naproxen sodium (Aleve®), and acetaminophen (Tylenol®). Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

The term “penetration enhancer” as used herein refers to an agent known to accelerate the delivery of a substance through the skin.

The term “pharmaceutically acceptable carrier” as used herein refers to any substantially non-toxic carrier conventionally useable for topical administration of pharmaceuticals in which the compound will remain stable and bioavailable when applied directly to skin or mucosal surfaces.

The term “pharmaceutical composition” as used herein refers to a composition that is employed to prevent, reduce in intensity, cure or otherwise treat a target condition.

The terms “pharmaceutically effective amount”, “cosmetically effective amount”, or “cosmeceutically effective amount as used herein refer to the amount of any of the compositions of the invention that result in a therapeutic or beneficial effect following its administration to a subject. The pharmaceutical, cosmeceutical, or cosmetic effect can be curing, minimizing, preventing or ameliorating a disease or disorder, improving the physical appearance and aesthetics, or may have any other pharmaceutical, cosmeceutical or cosmetic beneficial effect. The concentration of the substance is selected so as to exert its pharmaceutical, cosmeceutical or cosmetic effect, but low enough to avoid significant side effects within the scope and sound judgment of the skilled artisan. The effective amount of the composition may vary with the particular epithelial tissue being treated, the age and physical condition of the biological subject being treated, the severity of the condition, the duration of the treatment, the nature of concurrent therapy, the specific compound(s), composition or other active ingredient(s) employed, the particular carrier utilized, and like factors. A skilled artisan can determine a pharmaceutically effective amount of the inventive compositions by determining the unit dose. As used herein, a “unit dose” refers to the amount of inventive composition required to produce a response of 50% of maximal effect (i.e. ED₅₀). The unit dose can be assessed by extrapolating from dose-response curves derived from in vitro or animal model test systems.

The term “peptide” as used herein refers to a molecule of two or more amino acid chemically linked together. A peptide may refer to a polypeptide, protein or peptidomimetic.

The terms “polypeptide” and “protein” are used herein in their broadest sense to refer to a sequence of subunit amino acids, amino acid analogs, or peptidomimetics. The subunits are linked by peptide bonds, except where noted. The polypeptides described herein may be chemically synthesized or recombinantly expressed. Polypeptides of the described invention are chemically synthesized. Synthetic polypeptides, prepared using the well known techniques of solid phase, liquid phase, or peptide condensation techniques, or any combination thereof, can include natural and unnatural amino acids. Amino acids used for peptide synthesis may be standard Boc (N-α-amino protected N-α-t-butyloxycarbonyl)amino acid resin with the standard deprotecting, neutralization, coupling and wash protocols of the original solid phase procedure of Merrifield (1963, J. Am. Chem. Soc. 85:2149-2154), or the base-labile N-α-amino protected 9-fluorenylmethoxycarbonyl (Fmoc) amino acids first described by Carpino and Han (1972, J. Org. Chem. 37:3403-3409). Both Fmoc and Boc N-α-amino protected amino acids can be obtained from Sigma, Cambridge Research Biochemical, or other chemical companies familiar to those skilled in the art. In addition, the polypeptides can be synthesized with other N-α-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept. Protein Res. 35:161-214, or using automated synthesizers. The polypeptides of the invention may comprise D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Synthetic amino acids include ornithine for lysine, and norleucine for leucine or isoleucine. In addition, the polypeptides can have peptidomimetic bonds, such as ester bonds, to prepare peptides with novel properties. For example, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo. Accordingly, these terms also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. In some embodiments, the peptide is of any length or size.

Use herein of the terms “peptide”, “peptides”, “polypeptide”, “peptidomimetic” or “protein” should be taken to include reference to “derivatives” of such compounds, unless the context requires otherwise, and to include “prodrugs.”

The term “peptidomimetic” as used herein refers to a small protein-like chain designed to mimic a peptide. A peptidomimetic typically arises from modification of an existing peptide in order to alter the molecule's properties.

The term “postpartum” as used herein means of or noting the period of time following childbirth.

The term “prodrug” as used herein means a peptide or derivative which is in an inactive form and which is converted to an active form by biological conversion following administration to a subject.

The term “protective” as used herein is used in the broadest pharmacological sense to mean any agent that isolates the exposed surface of the skin or other membrane from harmful or annoying stimuli.

The term “refractory” as used herein refers to being unaffected, unresponsive, resistant or not fully responsive.

The term “rubefacient” as used herein refers to an agent that induces hyperemia, wherein hyperemia means an increased amount of blood in a body part or organ. Rubefaction, which is induced by rubefacients, results from increased circulation to an injured area and is accompanied by a feeling of comfort, warmth, itching and hyperesthesia.

The term “sclerosant” as used herein refers to an agent used as a chemical irritant injected into a vein in sclerotherapy. Examples of sclerosants include, but are not limited to, morrhuate sodium, sodium tetradecyl sulfate, laureth 9 and ethanolamine oleate.

The term “steroidal anti-inflammatory agent” as used herein refers to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

The term “solubilizing agents” as used herein refers to those substances that enable solutes to dissolve.

The term “surfactants” as used herein refers to surface-active substances, such as a detergent.

The term “telogen effluvium” as used herein refers to a form of nonscarring alopecia characterized by diffuse hair shedding.

The term “thickeners” as used herein refer to agents that make the composition of the described invention dense or viscous in consistency.

The term “treating” as used herein includes abrogating, substantially inhibiting, slowing or reversing the progression of a condition, substantially ameliorating clinical or aesthetical symptoms of a condition, substantially preventing the appearance of clinical or aesthetical symptoms of a condition, protecting from harmful or annoying stimuli or generally promoting healthy epithelial tissue.

The term “topical” as used herein refers to administration of an inventive composition at, or immediately beneath, the point of application.

The phrase “topically applying” describes application onto one or more surfaces(s) including epithelial surfaces. The composition may be applied by pouring, dropping, or spraying, if a liquid; rubbing on, if an ointment, lotion, cream, gel, or the like; dusting, if a powder; spraying, if a liquid or aerosol composition; or by any other appropriate means.

The term “vitamin” as used herein, refers to any of various organic substances essential in minute quantities to the nutrition of most animals. Vitamins act especially as coenzymes and precursors of coenzymes in the regulation of metabolic processes. Non-limiting examples of vitamins usable in context of the described invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like).

The term “vitaminize” (or “vitaminized”) as used herein refers to provide or supplement with vitamins. For example, the phrases “vitaminized peptides” or “vitaminized proteins” as used herein refer to peptides or proteins supplemented with or complexed with vitamins.

### Substituents

The term “aliphatic” as used herein, denotes a straight- or branched-chain arrangement of constituent carbon atoms, including, but not limited to paraffins (alkanes), which are saturated, olefins (alkenes, alkadienes, alka times, alkatetraenes, etc.), which are unsaturated, and acetylenes (alkynes), which contain a triple bond. Double and triple bonds may occur in the same compound. In complex structures, the chains may be branched or cross-linked.

The term “lower” as used herein refers to a group having between one and six carbons.

The term “alkyl” as used herein refers to a straight or branched chain monovalent hydrocarbon radical having, except where specifically indicated otherwise, from 1 to 25 carbon atoms, optionally substituted with substituents including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkyl” group may containing one or more O, S, S(O), or S(O)₂ moieties. Examples of “alkyl” as used herein include, but are not limited to, methyl, ethyl, propyl, decyl, undecyl, octadecyl, nonadecyl, eicosyl, heneicosyl, decosyl, tricosyl, tetracosyl, and pentacosyl, n-butyl, t-butyl, n-pentyl, isobutyl, and isopropyl, and the like. In some embodiments of the described invention, the prostaglandin analog has an alkyl of 1, 2, 3, 4, 5, 6, 7, 8, 9, 10, 11, or 12 carbon atoms.

The term “alkylene” as used herein refers to a straight or branched chain divalent hydrocarbon radical having from one to 25 carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkylene” group may containing one or more O, S, S(O), or S(O)₂ moieties. Examples of “alkylene” as used herein include, but are not limited to, methylene, ethylene, and the like.

The term “alkenyl,” as used herein, denotes a monovalent, straight (unbranched) or branched hydrocarbon chain having one or more double bonds therein where the double bond can be unconjugated or conjugated to another unsaturated group (e.g., a polyunsaturated alkenyl) and can be unsubstituted or substituted, with multiple degrees of substitution being allowed. It may be optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenyl” group may contain one or more O, S, S(O), or S(O)₂ moieties. For example, and without limitation, the alkenyl can be vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, decenyl, undecenyl, dodecenyl, heptadecenyl, octadecenyl, nonadecenyl, eicosenyl, heneicosenyl, docosenyl, tricosenyl, tetracisenyl, pentacosenyl, phytyl, the branched chain isomers thereof, and polyunsaturated alkenes including octadec-9,12,-dienyl, octadec-9,12,15-trienyl, and eicos-5,8,11,14-tetraenyl. Except where specifically indicated otherwise, any olefinic double bond of an alkenyl group that is capable of cis-trans isomerism may possess independently either the E or Z configuration.

As used herein, the term “alkenylene” refers to a straight or branched chain divalent hydrocarbon radical having from 2 to 25 carbon atoms and one or more carbon-carbon double bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenylene” group may containing one or more O, S, S(O), or S(O)₂ moieties. Examples of “alkenylene” as used herein include, but are not limited to, ethene-1,2-diyl, propene-1,3-diyl, methylene-1,1-diyl, and the like. Except where specifically indicated otherwise, any olefinic double bond of an alkenyl group that is capable of cis-trans isomerism may possess independently either the E or Z configuration.

As used herein, the term “alkynyl” refers to a hydrocarbon radical having from 2 to 25 carbons and at least one carbon-carbon triple bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynyl” group may containing one or more O, S, S(O), or S(O)₂ moieties.

As used herein, the term “alkynylene” refers to a straight or branched chain divalent hydrocarbon radical having from 2 to 25 carbon atoms and one or more carbon-carbon triple bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, amino sulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynylene” group may containing one or more O, S, S(O), or S(O)₂ moieties. Examples of “alkynylene” as used herein include, but are not limited to, ethyne-1,2-diyl, propyne-1,3-diyl, and the like.

The term “aryl” as used herein refers to an optionally substituted benzene ring or to an optionally substituted benzene ring system fused to one or more optionally substituted benzene rings, with multiple degrees of substitution being allowed. Substituents include, but are not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, amino sulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of aryl include, but are not limited to, phenyl, 2-napthyl, 1-naphthyl, 1-anthracenyl, and the like.

It should be understood that wherever the terms “alkyl” or “aryl” or either of their prefix roots appear in a name of a substituent, they are to be interpreted as including those limitations given above for alkyl and aryl. Designated numbers of carbon atoms (e.g., C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “arylene” refers to a benzene ring diradical or to a benzene ring system diradical fused to one or more optionally substituted benzene rings, wherein said benzene ring diradical or benzene ring system diradical is optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “arylene” include, but are not limited to, benzene-1,4-diyl, naphthalene-1,8-diyl, and the like.

The terms “carbamates” or “urethanes” as used herein refer to a group of organic compounds sharing a common functional group having the general structure —NH(CO)O—.

As used herein, “cycloalkyl” (used interchangeably with “aliphatic cyclic” herein) refers to a alicyclic hydrocarbon group optionally possessing one or more degrees of unsaturation, having from three to twelve carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. “Cycloalkyl” includes by way of example cyclopropyl, cyclobutyl, cyclopentyl, cyclohexyl, cycloheptyl, or cyclooctyl, and the like.

As used herein, the term “cycloalkylene” refers to an non-aromatic alicyclic divalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

The terms “heterocycle” and “heterocyclic” as used herein are used interchangeably to refer to a three to twelve-membered heterocyclic ring optionally aromatic or possessing one or more degrees of unsaturation, containing one or more heteroatomic substitutions selected from —S—, —SO—, —SO₂—, —O—, or —N—, optionally substituted with substitutents, including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring optionally may be fused to one or more of another “heterocyclic,” cycloalkyl or aryl ring(s). Examples of “heterocyclic” include, but are not limited to, pyrrole, furan, thiophene, imidazole, oxazole, thiazole, pyrazole, 3-pyrroline, pyrrolidine, pyridine, pyrimidine, purine, quinoline, isoquinoline, carbazole and the like.

The term C-linked heterocycle means a heterocycle that is bonded through a carbon atom, e.g. —(CH₂)ₙ-heterocycle where n is 1, 2 or 3 or —C<heterocycle where C< represents a carbon atom in a heterocycle ring. Similarly, R moieties that are N-linked heterocycles mean a heterocycle that is bonded through a heterocycle ring nitrogen atom, e.g. —N<heterocycle where N< represents a nitrogen atom in a heterocycle ring. A variable group such as an R moiety that is bonded to a Formula I, II, III, IV, or V compound can be a C-linked heterocycle or a N-linked heterocycle, These heterocycles include those listed below or described elsewhere herein.

Examples of heterocycles include by way of example and not limitation pyridyl, thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathiinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4H-carbazolyl, carbazolyl, .beta.-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at the nitrogen atom or position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Typically, nitrogen bonded heterocycles include 1-aziridyl, 1-azetedyl, 1-pyrrolyl, 1-imidazolyl, 1-pyrazolyl, and 1-piperidinyl and structures such as and tautomers of any of these.

As used herein, the term “heterocyclylene” refers to a three to twelve-membered heterocyclic ring diradical optionally having one or more degrees of unsaturation containing one or more heteroatoms selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-diyl, and the like.

As used herein, the term “heteroaryl” refers to a five-to seven-membered aromatic ring, or to a polycyclic heterocyclic aromatic ring, containing one or more nitrogen, oxygen, or sulfur heteroatoms, where N-oxides and sulfur monoxides and sulfur dioxides are permissible heteroaromatic substitutions, optionally substituted with substituents including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring systems, one or more of the rings may contain one or more heteroatoms. Examples of “heteroaryl” used herein are furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like.

As used herein, the term “heteroarylene” refers to a five-to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

The term “hydrate” as used herein refers to a compound formed by the addition of water or its elements to another molecule. The water usually can split off by heating, yielding the anhydrous compound.

The term “isomer” as used herein refers to one of two or more molecules having the same number and kind of atoms and hence the same molecular weight, but differing in chemical structure. Isomers may differ in the connectivities of the atoms (structural isomers), or they may have the same atomic connectivities but differ only in the arrangement or configuration of the atoms in space (stereoisomers). Stereoisomers may include, but are not limited to, E/Z double bond isomers, enantiomers, and diastereomers. Structural moieties that, when appropriately substituted, can impart stereoisomerism include, but are not limited to, olefinic, imine or oxime double bonds; tetrahedral carbon, sulfur, nitrogen or phosphorus atoms; and allenic groups. Enantiomers are non-superimposable mirror images. A mixture of equal parts of the optical forms of a compound is known as a racemic mixture or racemate. Diastereomers are stereoisomers that are not mirror images. The invention provides for each pure stereoisomer of any of the compounds described herein. Such stereoisomers may include enantiomers, diastereomers, or E or Z alkene, imine or oxime isomers. The invention also provides for stereoisomeric mixtures, including racemic mixtures, diastereomeric mixtures, or E/Z isomeric mixtures. Stereoisomers can be synthesized in pure form (Nógrádi, M.; Stereoselective Synthesis, (1987) VCH Editor Ebel, H. and Asymmetric Synthesis, Volumes 3-5, (1983) Academic Press, Editor Morrison, J.) or they can be resolved by a variety of methods such as crystallization and chromatographic techniques (Jaques, J.; Collet, A.; Wilen, S.; Enantiomer, Racemates, and Resolutions, 1981, John Wiley and Sons and Asymmetric Synthesis, Vol. 2, 1983, Academic Press, Editor Morrison, J). In addition the compounds of the described invention may be present as enantiomers, diasteriomers, isomers or two or more of the compounds may be present to form a racemic or diastereomeric mixture. As used herein, the term “fused cycloalkylaryl” refers to a cycloalkyl group fused to an aryl group, the two having two atoms in common, and wherein the aryl group is the point of substitution. Examples of “fused cycloalkylaryl” used herein include, but are not limited to, 5-indanyl, 5,6,7,8-tetrahydro-2-naphthyl,

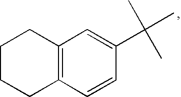

and the like.

As used herein, the term “fused cycloalkylarylene” refers to a fused cycloalkylaryl, wherein the aryl group is divalent. Examples include, but are not limited to,

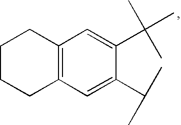

and the like.

As used herein, the term “fused arylcycloalkyl” refers to an aryl group fused to a cycloalkyl group, the two having two atoms in common, and wherein the cycloalkyl group is the point of substitution. Examples of “fused arylcycloalkyl” used herein include, but are not limited to, 1-indanyl, 2-indanyl, 1-(1,2,3,4-tetrahydronaphthyl),

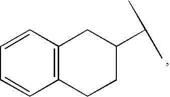

and the like.

As used herein, the term “fused arylcycloalkylene” refers to a fused arylcycloalkyl, wherein the cycloalkyl group is divalent. Examples include, but are not limited to,

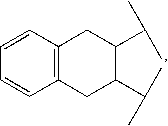

and the like.

As used herein, the term “fused heterocyclylaryl” refers to a heterocyclyl group fused to an aryl group, the two having two atoms in common, and wherein the aryl group is the point of substitution. Examples of “fused heterocyclylaryl” used herein include, but are not limited to, 3,4-methylenedioxy-1-phenyl,

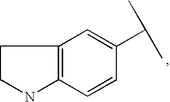

and the like

As used herein, the term “fused heterocyclylarylene” refers to a fused heterocyclylaryl, wherein the aryl group is divalent. Examples include, but are not limited to,

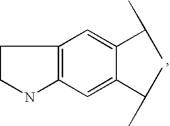

and the like.

As used herein, the term “fused arylheterocyclyl” refers to an aryl group fused to a heterocyclyl group, the two having two atoms in common, and wherein the heterocyclyl group is the point of substitution. Examples of “fused arylheterocyclyl” used herein include, but are not limited to, 2-(1,3-benzodioxolyl),

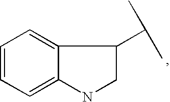

and the like.

As used herein, the term “fused arylheterocyclylene” refers to a fused arylheterocyclyl, wherein the heterocyclyl group is divalent. Examples include, but are not limited to,

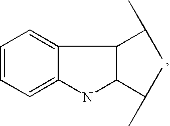

and the like.

As used herein, the term “fused cycloalkylheteroaryl” refers to a cycloalkyl group fused to a heteroaryl group, the two having two atoms in common, and wherein the heteroaryl group is the point of substitution. Examples of “fused cycloalkylheteroaryl” used herein include, but are not limited to, 5-aza-6-indanyl,

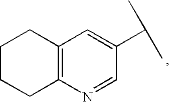

and the like.

As used herein, the term “fused cycloalkylheteroarylene” refers to a fused cycloalkylheteroaryl, wherein the heteroaryl group is divalent. Examples include, but are not limited to,

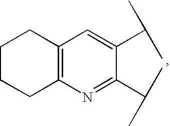

and the like.

As used herein, the term “fused heteroarylcycloalkyl” refers to a heteroaryl group fused to a cycloalkyl group, the two having two atoms in common, and wherein the cycloalkyl group is the point of substitution. Examples of “fused heteroarylcycloalkyl” used herein include, but are not limited to, 5-aza-1-indanyl,

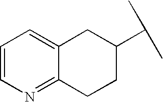

and the like.

As used herein, the term “fused heteroarylcycloalkylene” refers to a fused heteroarylcycloalkyl, wherein the cycloalkyl group is divalent. Examples include, but are not limited to,

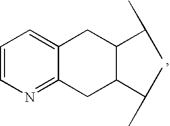

and the like.

As used herein, the term “fused heterocyclylheteroaryl” refers to a heterocyclyl group fused to a heteroaryl group, the two having two atoms in common, and wherein the heteroaryl group is the point of substitution. Examples of “fused heterocyclylheteroaryl” used herein include, but are not limited to, 1,2,3,4-tetrahydro-beta-carbolin-8-yl,

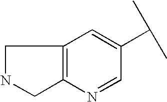

and the like.

As used herein, the term “fused heterocyclylheteroarylene” refers to a fused heterocyclylheteroaryl, wherein the heteroaryl group is divalent. Examples include, but are not limited to,

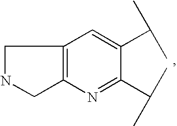

and the like.

As used herein, the term “fused heteroarylheterocyclyl” refers to a heteroaryl group fused to a heterocyclyl group, the two having two atoms in common, and wherein the heterocyclyl group is the point of substitution. Examples of “fused heteroarylheterocyclyl” used herein include, but are not limited to, -5-aza-2,3-dihydrobenzofuran-2-yl,

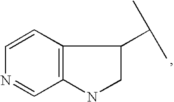

and the like.

As used herein, the term “fused heteroaryiheterocyclylene” refers to a fused heteroarylheterocyclyl, wherein the heterocyclyl group is divalent. Examples include, but are not limited to,

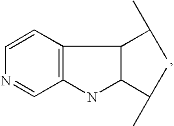

and the like.

As used herein, the term “acid isostere” refers to a substituent group which will ionize at physiological pH to bear a net negative charge. Examples of such “acid isosteres” include but are not limited to heteroaryl groups such as but not limited to isoxazol-3-ol-5-yl, 1H-tetrazole-5-yl, or 2H-tetrazole-5-yl. Such acid isosteres include but are not limited to heterocyclyl groups such as but not limited to imidazolidine-2,4-dione-5-yl, imidazolidine-2,4-dione-1-yl, 1,3-thiazolidine-2,4-dione-5-yl, or 5-hydroxy-4,1-pyran-4-on-2-yl.

As used herein, the term “direct bond” or “covalent bond”, where part of a structural variable specification, refers to the direct joining of the substituents flanking (preceding and succeeding) the variable taken as a “direct bond”.

The term “O-linked moiety” means a moiety that is bonded through an oxygen atom. Thus, when an R group is an O-linked moiety, that R is bonded through oxygen and it can thus be an ether, an ester (e.g., —O—C(O)-optionally substituted alkyl), a carbonate or a carbamate (e.g., —O—C(O)—NH₂ or —O—C(O)—NH-optionally substituted alkyl). Similarly, the term “S-linked moiety” means a moiety that is bonded through a sulfur atom. Thus, when an R group is an S-linked moiety, that R is bonded through sulfur and it can thus be a thioether (e.g., —S-optionally substituted alkyl), a sulfoxide (e.g., —S(O)-optionally asubstituted alkyl), a sulfone (e.g., —S(O)₂-optionally substituted alkyl)ₐ thioester (—S—C(O)-optionally substituted alkyl) or a disulfide (e.g., —S—S-optionally substituted alkyl). The term “N-linked moiety” means a moiety that is bonded through a nitrogen atom. Thus, when an R group is an N-linked moiety, the R group is bonded through nitrogen and one or more of these can thus be an N-linked amino acid such as —NH—CH₂—COOH, a carbamate such as —NHC(O)—O-optionally substituted alkyl, an amine such as —NH-optionally substituted alkyl, an amide such as —NH—C(O)-optionally substituted alkyl or an azide —N₃. The term “C-linked moiety” means a moiety that is bonded through a carbon atom. When one or more R group is bonded through carbon, one or more of these can thus be -optionally substituted alkyl such as —CH₂—CH₂—O—CH₃, —C(O)-optionally substituted alkyl hydroxyalkyl, mercaptoalkyl, aminoalkyl or ═CH-optionally substituted alkyl.

The term “alkoxy” as used herein refers to the group RₐO—, where Rₐ is alkyl.

The term “alkenyloxy” as used herein refers to the group RₐO—, where Rₐ is alkenyl.

The term “alkynyloxy” as used herein refers to the group RₐO—, where Rₐ is alkynyl.

The term “alkylsulfanyl” as used herein refers to the group RₐS—, where Rₐ is alkyl.

The term “alkenylsulfanyl” as used herein refers to the group RₐS—, where Rₐ is alkenyl.

The term “alkynylsulfanyl” as used herein refers to the group RₐS—, where Rₐ is alkynyl.

The term “alkylsulfenyl” as used herein refers to the group RₐS(O)—, where Rₐ is alkyl.

The term “alkenylsulfenyl” as used herein refers to the group RₐS(O)—, where Rₐ is alkenyl.

The term “alkynylsulfenyl” as used herein refers to the group RₐS(O)—, where Rₐ is alkynyl.

The term “alkylsulfonyl” as used herein refers to the group RₐSO₂—, where Rₐ is alkyl.

The term “alkenylsulfonyl” as used herein refers to the group RₐSO₂—, where Rₐ is alkenyl.

The term “alkynylsulfonyl” as used herein refers to the group RₐSO₂—, where Rₐ is alkynyl.

The term “acyl” as used herein refers to the group RₐC(O)—, where Rₐ is alkyl, alkenyl, alkynyl, cycloalkyl, cycloalkenyl, or heterocyclyl.

The term “aroyl” as used herein refers to the group RₐC(O)—, where Rₐ is aryl.

The term “heteroaroyl” as used herein refers to the group RₐC(O)—, where Rₐ is heteroaryl.

The term “alkoxycarbonyl” as used herein refers to the group RₐOC(O)—, where Rₐ is alkyl.

The term “acyloxy” as used herein refers to the group RₐC(O)O—, where Rₐ is alkyl, alkenyl, alkynyl, cycloalkyl, cycloalkenyl, or heterocyclyl.

The term “aroyloxy” as used herein refers to the group RₐC(O)O—, where Rₐ is aryl.

The term “heteroaroyloxy” as used herein refers to the group RₐC(O)O—, where Rₐ is heteroaryl.

The term “substituted” as used herein refers to substitution with the named substituent or substituents, multiple degrees of substitution being allowed unless otherwise stated.

The terms “contain” or “containing” as used herein refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

The term “oxo” as used herein refers to the substituent ═O.

The term “halogen” or “halo” as used herein includes iodine, bromine, chlorine and fluorine.

The term “mercapto” as used herein refers to the substituent —SH.

The term “carboxy” as used herein refers to the substituent —COOH.

The term “cyano” as used herein refers to the substituent —CN.

The term “aminosulfonyl” as used herein refers to the substituent —SO₂ NH₂.

The term “carbamoyl” as used herein refers to the substituent —C(O)NH₂.

The term “sulfanyl” as used herein refers to the substituent —S—.

The term “sulfenyl” as used herein refers to the substituent —S(O)—.

The term “sulfonyl” as used herein refers to the substituent —S(O)₂—.

The term “ethoxy” as used herein refers to the substituent —O—CH₂CH₃.

The term “methoxy” as used herein refers to the substituent —O—CH₃.

As used herein, the term “optionally” means that the subsequently described event(s) may or may not occur, and includes both event(s) which occur and events that do not occur.

The term “solvate” as used herein refers to a complex formed by the attachment of solvent molecules to that of a solute. The term “solvent” refers to a substance capable of dissolving another substance (termed a “solute”) to form a uniformly dispersed mixture (solution).

The term “carbohydrate” as used herein refers to aldehyde or ketone compounds with multiple hydroxyl groups. The term “monosaccharide” or “simple sugar” refers to a carbohydrate that does not hydrolyze. Non-limiting examples of monosaccharides include glucose (dextrose), fructose, galactose, xylose and ribose. Monosaccharides are the building blocks of disaccharides like sucrose (common sugar) and polysaccharides (such as cellulose and starch). Further, each carbon atom that supports a hydroxyl group (except for the first and last) is chiral, giving rise to a number of isomeric forms all with the same chemical formula.

The term “configuration” refers to the three-dimensional shape of a molecule. In order to represent three-dimensional configurations on a two-dimensional surface, perspective drawings in which the direction of a bond is specified by the line connecting the bonded atoms are used. Formula A1 shows an illustrative perspective drawing:

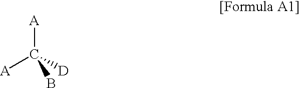

In formula A1, the focus of configuration is a carbon (C) atom so the lines specifying bond directions will originate there. A simple straight line represents a bond lying approximately in the surface plane, as shown by the two bonds to substituent “A.” A wedge shaped bond is directed in front of this plane (thick end toward the viewer), as shown by the bond to substituent “B.” A hatched bond is directed in back of the plane (away from the viewer), as shown by the bond to substituent “D.” A dashed line represents a single or double bond which can be in the E or Z configuration.

### Compositions for Treating Epithelial-Related Disorders

According to one aspect, the described invention provides compositions for treating epithelial-related disorders, the composition comprising a first component, wherein the first component is at least one prostaglandin analog; and optionally, a second component, wherein the second component is at least one imidazole analog; and a carrier.

### Prostaglandin Analogs

In one aspect, the invention provides a topical composition for treating an epithelial-related condition comprising (a) at least one compound of Formula I or a pharmaceutically acceptable salt, hydrate, solvate, prodrug or metabolite thereof,

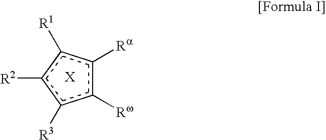

wherein ring X is selected from

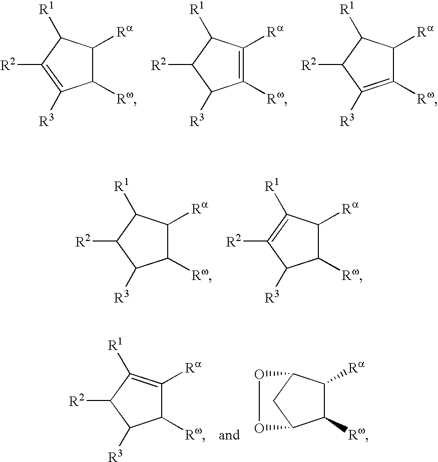

wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each,

wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —(PO₂OH)₅H where s is 1˜25 or a pharmaceutically acceptable salt thereof; —P(O)(OH)₂ or a pharmaceutically acceptable salt thereof,

wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁷ groups,

wherein R^{α} is

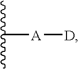

wherein A is a divalent hydrocarbon radical having from two to ten carbon atoms, which may be interrupted by one or more —O— or —S—, zero or one 1,2-phenylene, 1,3-phenylene, or 1,4-phenylene radical, and zero or one

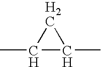

in either cis or trans configuration; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof,

wherein each R⁷ is independently H, F, or straight chain or branched C₁˜Cₛ alkyl,

wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁷ groups,

wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; —NO₂; —SR¹⁰; —CH═NOR¹⁰; —C(═O)NR¹¹OR¹²; —S(O)₂ NR⁹₂; —NR⁹S(O)₂R¹³; —OS(O)₂ NR⁹₂; —C(O)NHS(O)₂R¹³; —S(O)₂R¹³; —SO₃H; —PO₃H₂;

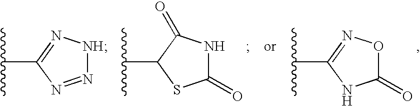

wherein R^{ω} is

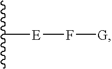

wherein E is a divalent hydrocarbon radical having from two to ten carbon atoms, which may be interrupted by one or more —O— or —S— and zero or one 1,2-phenylene, 1,3-phenylene, or 1,4-phenylene radical; said hydrocarbon radical containing zero to four C═C or C≡C bonds and zero to one C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof,

wherein F is —CH₂—, —O—; —S—; —S(O)—; —S(O₂)—; —C(O)—; —C(O)O—; —C(O)S—; —C(O)NR⁹—; —NR⁹—; or a covalent bond,

wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁵ groups,

wherein each R⁸ is independently selected from the group consisting of: H; a pharmaceutically acceptable cation including, but not limited to, sodium, potassium, magnesium, calcium or an organic cation including but not limited to an ammonium ion; a C₁˜C₂₀ straight chain or branched acyclic hydrocarbon group, which may be interrupted by one or more —O— or —S—, said hydrocarbon group containing zero to four C═C or C≡C bonds wherein each C═C bond independently may be of E or Z configuration, said hydrocarbon group having no two heteroatoms adjacent and no heteroatom adjacent to a non-aromatic C═C or C≡C bond, said hydrocarbon group being substituted with zero to four R¹⁵ groups; —(CH₂)_{q}OH, —(CH₂)OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including but not limited to a lower alkyl ester, a lower acyloxy-alkyl ester (including, but not limited to, acetoxymethyl, acetoxyethyl, aminocarbonyloxymethyl, pivaloyloxymethyl or pivaloyloxyethyl ester), a lactonyl ester (including, but not limited to, a phthalidyl or thiophthalidyl ester), a lower alkoxyacyloxyalkyl ester (including, but not limited to, a methoxycarbonyloxymethyl, ethoxycarbonyloxyethyl or isopropoxycarbonyloxyethyl ester), an alkoxyalkyl ester, choline ester or acylamino alkyl ester (including, but not limited to, an acetamidomethyl ester); or -J-K, wherein J is a covalent bond or a C₁-C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁵ groups,

wherein each R⁹ is independently selected from the group consisting of: H; a C₁˜C₂₀ straight chain or branched acyclic hydrocarbon group containing zero to four C═C or C≡C bonds wherein each C═C bond independently may be of E or Z configuration; a C₁˜C₂₀ straight chain or branched acyl group containing zero to four C═C or C≡C bonds wherein each C═C bond independently may be of E or Z configuration; —(CH₂)_{q}OH, —(CH₂)OR¹⁴, —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl (including, but not limited to, acetoxymethyl, acetoxyethyl, aminocarbonyloxymethyl, pivaloyloxymethyl or pivaloyloxyethyl), lactonyl (including, but not limited, to a phthalidyl or thiophthalidyl), lower alkoxyacyloxyalkyl (including, but not limited to, a methoxycarbonyloxymethyl, ethoxycarbonyloxyethyl or isopropoxycarbonyloxyethyl), or acylamino alkyl (including, but not limited to, acetamidomethyl); or -J-K; or —NR⁹₂ may be a cycloamido radical (including, but not limited to, 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which may be alike or different, or 1-piperazinyl substituted at the 4-position by R⁹, and the like),

wherein each R¹⁰ is independently H; a C₁˜C₂₀ straight chain or branched acyclic hydrocarbon group containing zero to four C═C or C≡C bonds wherein each C═C bond independently may be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl

wherein each R¹¹ is independently H or —C(O)R¹⁶,

wherein each R¹² is independently R¹⁶ or —C(O)R¹⁶,

wherein each R¹³ is independently a C₁˜C₂₀ straight chain or branched acyclic hydrocarbon group, which may be interrupted by one or more —O— or —S—, said hydrocarbon group containing zero to four C═C or C≡C bonds wherein each C═C bond independently may be of E or Z configuration, said hydrocarbon group having no two heteroatoms adjacent and no heteroatom adjacent to a non-aromatic C═C or C≡C bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or -L-O-M (“O” being oxygen),

wherein each R¹⁴ is independently straight chain or branched C₁˜C₆ alkyl or —CH₂OCH₃,

wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; —CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C N; —N₃; —CF₂OH; —NO₂; —SR¹⁰; —CH═NOR¹⁰; —CH═N—NH—C(O)—NH₂; —C(═O)NR¹¹OR¹²; —S(O)₂ N(R¹⁶)₂; —NR¹⁶S(O)₂R¹³; —C(O)NHS(O)₂R¹³; —S(O)₂R¹³; —SO₃H; —PO₃H₂;

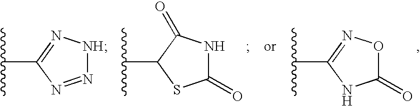

wherein each R¹⁶ is independently H or straight chain or branched C₁˜C₆ alkyl, phenyl or —CH₂OCH₃,

wherein each R¹⁷ is independently straight chain or branched C₁ C₆ alkyl; —C(O)OR¹⁶; phenyl; F; Cl; Br; I; —CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂N(R₁₆)₂; —NR¹⁶S(O)₂R¹³, with the proviso that, if R¹⁷ is —NR¹⁶S(O)₂R¹³, R', R², R³, R^{α} and R^{ω} are selected such that the molecular weight of the compound of Formula I does not exceed about 2000 atomic mass units, and

wherein not more than four of R⁷ are other than H or F and not more than four of R⁷ are F; and

wherein each chiral center or allenic moiety independently possesses any relative or absolute stereoconfiguration or comprises any mixture thereat and

(b) a carrier; wherein the composition stimulates hair growth on an epithelial surface to which the composition has been applied.

In some embodiments, the at least one compound of Formula I is diastereomerically pure.

In some embodiments, the at least one compound of Formula I is a mixture of diastereomers in any ratio.

In some embodiments, the at least one compound of Formula I is enantiomerically pure.

In some embodiments, the at least one compound of Formula I is a mixture of enantiomers in any ratio, including a racemate.

In some embodiments, the at least one compound of Formula I is diastereomerically and enantiomerically pure.

In some embodiments, the at least one compound of Formula I is a mixture of diastereomers and enantiomers in any ratio.

In some embodiments, the at least one compound of Formula I has one or more hydrogen atoms replaced by deuterium.

In another aspect, the invention provides a topical composition for treating an epithelial-related condition comprising (a) at least one compound of Formula II or a pharmaceutically acceptable salt, hydrate, solvate, prodrug or metabolite thereof,

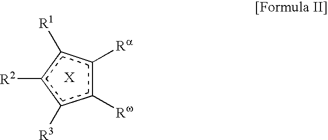

wherein ring X is selected from

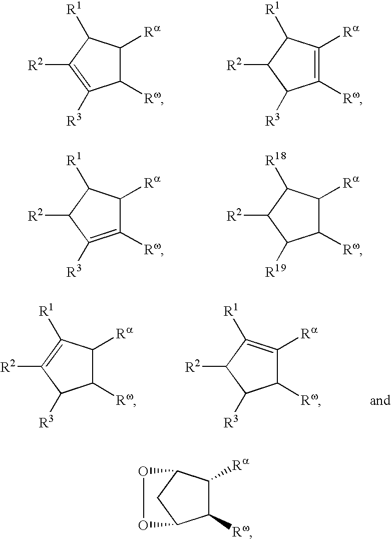

wherein R¹, R², R³, R¹⁸ and R¹⁹ are independently H, —OR⁴, ═O, or —OC(O)R⁵, where the carbon atoms to which R¹, R², R³, R¹⁸ and R¹⁹ attach bear the appropriate number of additional H atoms so as to have exactly 4 bonds each, with the proviso that, when R¹⁸ is —OR⁴ or —OC(O)R⁵ and is cis to R^{α} and trans to R^{ω} with respect to the plane of the cyclopentane ring, then R¹⁹ is H or ═O,

wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —(PO₂OH)ₛH where s is 1-25 or a pharmaceutically acceptable salt thereof; or —P(O)(OH)₂ or a pharmaceutically acceptable salt thereof,

wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₁˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁷ groups,

wherein R^{α} is

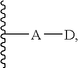

wherein A is a divalent hydrocarbon radical having from two to ten carbon atoms, which may be interrupted by one or more —O— or —S—, zero or one 1,2-phenylene, 1,3-phenylene, or 1,4-phenylene radical, and zero or one

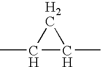

in either cis or trans configuration; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, O(CO)R⁵, R⁷ or M groups; wherein each olefinic moiety may independently be E or Z and each allenic moiety or chiral center may independently possess any relative or absolute stereoconfiguration or any mixture thereof,

wherein each R⁷ is independently H, F, or straight chain or branched C₁˜C₅ alkyl,

wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁷ groups,

wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; —NO₂; —SR¹⁰; —CH═NOR¹⁰; —C(═O)NR¹¹OR¹²; —S(O)₂ NR⁹₂; —NR⁹S(O)₂R¹³; OS(O)₂ NR⁹₂; —C(O)NHS(O)₂R¹³; —S(O)₂R¹³; —SO₃H; —PO₃H₂;

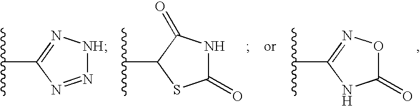

wherein R^{ω} is

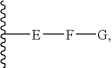

wherein E is a divalent hydrocarbon radical having from two to ten carbon atoms, which may be interrupted by one or more —O— or —S— and zero or one 1,2-phenylene, 1,3-phenylene, or 1,4-phenylene radical; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof,

wherein F is —CH₂, —O—; —S—; —S(O)—; —S(O₂); —C(O)—; —C(O)O—; —C(O)S—; —C(O)NR⁹—; —NR⁹—; or a covalent bond,

wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁵ groups,

wherein each R⁸ is independently selected from the group consisting of: H; a pharmaceutically acceptable cation including, but not limited to, sodium, potassium, magnesium, calcium or an organic cation including, but not limited to, an ammonium ion; a C₁˜C₂₀ straight chain or branched acyclic hydrocarbon group, which may be interrupted by one or more —O— or —S—, said hydrocarbon group containing zero to four C═C or bonds wherein each C═C bond independently may be of E or Z configuration, said hydrocarbon group having no two heteroatoms adjacent and no heteroatom adjacent to a non-aromatic C═C or C═C bond, said hydrocarbon group being substituted with zero to four R¹⁵ groups; —(CH₂)₄OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including, but not limited to, a lower alkyl ester, a lower acyloxy-alkyl ester (including but not limited to acetoxymethyl, acetoxyethyl, aminocarbonyloxymethyl, pivaloyloxymethyl or pivaloyloxyethyl ester), a lactonyl ester (including, but not limited to, a phthalidyl or thiophthalidyl ester), a lower alkoxyacyloxyalkyl ester (including, but not limited to, a methoxycarbonyloxymethyl, ethoxycarbonyloxyethyl or isopropoxycarbonyloxyethyl ester), an alkoxyalkyl ester, choline ester or acylamino alkyl ester (including, but not limited to, an acetamidomethyl ester); or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁵ groups,

wherein each R⁹ is independently selected from the group consisting of: H; a C₁˜C₂₀ straight chain or branched acyclic hydrocarbon group containing zero to four C═C or C═C bonds wherein each C═C bond independently may be of E or Z configuration; a C₁˜C₂₀ straight chain or branched acyl group containing zero to four C═C or C≡C bonds wherein each C═C bond independently may be of E or Z configuration; —(CH₂)_{q}OH, —(CH₂)_{q}OR¹⁴, —(CH₂)_{q}OC(O)R¹⁴, —(CH₂)_{q}CN, —(CH₂)_{q}CO₂H, —(CH₂)_{q}OC(O)NH₂, or —(CF₁₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl (including, but not limited to, acetoxymethyl, acetoxyethyl, aminocarbonyloxymethyl, pivaloyloxymethyl or pivaloyloxyethyl), lactonyl (including, but not limited to, a phthalidyl or thiophthalidyl), lower alkoxyacyloxyalkyl (including, but not limited to, a methoxycarbonyloxymethyl, ethoxycarbonyloxyethyl or isopropoxycarbonyloxyethyl), or acylamino alkyl (including, but not limited to, acetamidomethyl); or -J-K; or —NR⁹₂ may be a cycloamido radical (including, but not limited to, 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which may be alike or different, or 1-piperazinyl substituted at the 4-position by R⁹, and the like),

wherein each R¹⁰ is independently H; a C₁˜C₂₀ straight chain or branched acyclic hydrocarbon group containing zero to four C═C or C≡C bonds wherein each C═C bond independently may be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl,

wherein each R¹¹ is independently H or —C(O)R¹⁶,

wherein each R¹² is independently R¹⁶ or —C(O)R¹⁶,

wherein each R¹³ is independently a C₁˜C_{H} straight chain or branched acyclic hydrocarbon group, which may be interrupted by one or more —O— or —S—, said hydrocarbon group containing zero to four C═C or C≡C bonds wherein each C═C bond independently may be of E or Z configuration, said hydrocarbon group having no two heteroatoms adjacent and no heteroatom adjacent to a non-aromatic or C≡C bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or -L-O-M (“O” being oxygen),

wherein each R¹⁴ is independently straight chain or branched C₁˜C₆ alkyl or —CH₂OCH₃,

wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; 1; —CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂ N(R¹⁶)₂; —NR¹⁶S(O)₂R¹³; —C(O)NHS(O)₂R¹³; —S(O)₂R¹³; —SO₃H; —PO₃H₂;

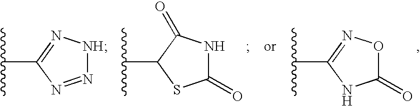

wherein each R¹⁶ is independently H or straight chain or branched C₁˜C₆ alkyl, phenyl or —CH₂OCH₃,

wherein each R¹⁷ is independently straight chain or branched C₁˜C₆ alkyl; —C(O)OR¹⁶; phenyl; F; Cl; Br; I; —CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂ N(R¹⁶)₂; —NR¹⁶S(O)₂R¹³, with the proviso that, if R¹⁷ is —NR¹⁶S(O)₂R¹³, R¹, R², R³, R¹⁸, R¹⁹, R^{α} and R^{ω} are selected such that the molecular weight of the compound of Formula II does not exceed about 2000 atomic mass units,

wherein not more than four of R⁷ are other than H or F and not more than four of R⁷ are F,

wherein each chiral center or allenic moiety independently may possess any relative or absolute stereoconfiguration or be any mixture thereof, unless specified otherwise herein; and

(b) a carrier; wherein the composition stimulates hair growth on an epithelial surface to which the composition has been applied.

In some such embodiments, the at least one compound of Formula II is diastereomerically pure.

In some such embodiments, the at least one compound of Formula II is a mixture of diastereomers in any ratio.

In some such embodiments, the at least one compound of Formula II is enantiomerically pure.

In some such embodiments, the at least one compound of Formula II is a mixture of enantiomers in any ratio, including a racemate.

In some such embodiments, the at least one compound of Formula II is diastereomerically and enantiomerically pure.

In some such embodiments, the at least one compound of Formula II is a mixture of diastereomers and enantiomers in any ratio.

In some such embodiments, the at least one compound of Formula II has one or more hydrogen atoms replaced by deuterium.

In some embodiments, when R² is H, G is phenyl or heterocycle and D is —C(O)NR⁹₂ or —C(O)OR⁸, the compound of the described invention is a compound of Formula (III) or a solvate, hydrate, salt, prodrug or metabolite of Formula (III):

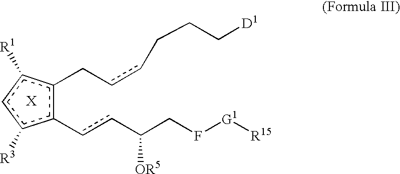

wherein G¹ is phenyl or a 6-membered heterocycle; said heterocycle being aromatic or non-aromatic; said heterocycle containing at least two heteroatoms selected from the group consisting of N, S and O; said phenyl or heterocycle being optionally substituted with one to three R¹⁵ groups;

wherein D¹ is —C(O)OR^{S} or —C(O)NR⁹₂.

The following are nonlimiting embodiments depicting cyclopentane ring X of the compound of Formula III, wherein R¹ is ═O and R³ is H, which has the structure:

The following are nonlimiting embodiments depicting cyclopentane ring X of the compound of Formula III, wherein R' is ═O and R³ is H, which has the structure:

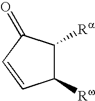

According to one embodiment, R⁵ is H, D¹ is —C(O)NR⁹₂, R⁹₂ is H and a nitrogen-linked cyclopropyl group, F is —O—, G¹ is phenyl and there is no R¹⁵. This compound of Formula III has the structure:

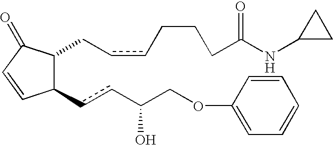

wherein a hashed line represents a substituent behind the plane of this paper, a bold wedge represents a substituent above the plane of this paper, and a dashed line represents a single or double bond which can be in the cis or trans configuration. In one embodiment, the compound in which the dashed lines are double bonds with the stereoconfigurations as drawn is 16-phenoxy 17,18,19,20-tetranor PGA₂ N-cyclopropylamide. A compound where the C₅-C₆ dashed line is a single bond, and the C₁₃-C₁₄ dashed line is a double bond with the stereoconfigurations as drawn is 16-phenoxy 17,18,19,20-tetranor PGA₁ N-cyclopropylamide.

According to another embodiment, R⁵ is H, D¹ is —C(O)NR⁹₂, R⁹₂ is H and a nitrogen-linked cyclopropylmethyl group, F is —O—, G¹ is phenyl and there is no R¹⁵. This compound of Formula III has the structure:

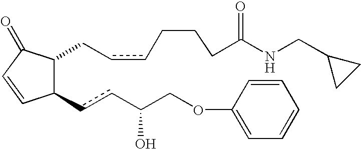

wherein a hashed line represents a substituent behind the plane of this paper, a bold wedge represents a substituent above the plane of this paper, wherein the dashed lines represent a single bond or a double bond which can be in the cis or trans configuration; wherein when the dashed lines represent a double bond and the 5, 6 bond is in a cis configuration and the 13, 14 bond is in a trans configuration, a compound with the stereoconfigurations as drawn is 16-phenoxy-17,18,19,20-tetranor PGA₂ N-cyclopropylmethylamide. A compound where the C₅-C₆ dashed line is a single bond, and the C₁₃-C₁₄ dashed line is a double bond with the stereoconfigurations as drawn is 16-phenoxy 17,18,19,20-tetranor PGA₁ N-cyclopropyl methylamide.

According to another embodiment, R⁵ is H, D¹ is —C(O)NR⁹₂, R⁹₂ is H and a nitrogen-linked cyclopropyl group, F is —CH₂—, G¹ is phenyl and there is no R¹⁵. This compound of Formula II has the structure:

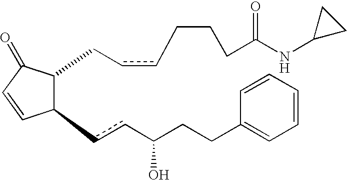

wherein a hashed line represents a substituent below the plane of this paper, a bold wedge represents a substituent above the plane of this paper, the dashed lines represent a single or a double bond; wherein when the dashed lines represent a double bond which can be in the cis or trans configuration. A compound with the stereoconfigurations as drawn is 17-phenyl-18,19,20 trinor PGA₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGA₁ N-cyclopropylamide.

According to another embodiment, R⁵ is H, D¹ is —C(O)NR⁹₂, R⁹₂ is H and a nitrogen-linked cyclopropylmethyl group, F is —CH₂—, G¹ is phenyl and there is no R¹⁵. This compound of Formula III has the structure:

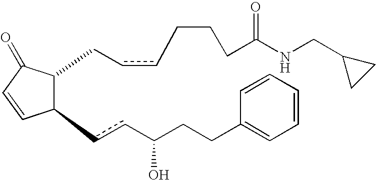

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfigurations as drawn is 17-phenyl-18,19,20-trinor PGA₂ N-cyclopropylmethylamide. A compound where the C₅-C₆ dashed line is a single bond, and the C₁₃-C₁₄ dashed line is a double bond with the stereoconfigurations as drawn is 17-phenyl-18,19,20-trinor PGA_{I} N-cyclopropylmethylamide.

According to another embodiment, R⁵ is H, D¹ is —C(O)NR⁹₂, R⁹₂ is H and a nitrogen-linked —CH(CH₂OH)₂ group, F is —CH₂—, G^{I} is phenyl and there is no R¹⁵. This compound of Formula III has the structure:

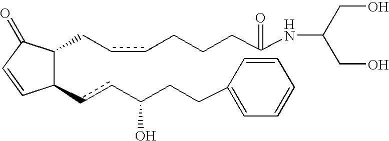

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfiguration as drawn is 17-phenyl-18,19,20-trinor PGA₂ (N-(1,3-dihydroxypropan-2-yl))amide. A compound wherein the C₅-C₆ dashed line is a single bond and the C₁₃-C₁₄ dashed line is a double bond with the stereoconfigurations as drawn is 17-phenyl-18,19,20-trinor PGA₁ (N-(1,3-dihydroxypropan-2-yl))amide

The following are nonlimiting embodiments depicting cyclopentane ring X of the compound of Formula III, wherein R¹ is ═O, and R³ is H, which has the structure:

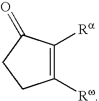

In some embodiments, R⁵ is H, D¹ is —C(O)NR⁹₂, R⁹₂ is H and a nitrogen-linked-cyclopropyl group, F is —O—, G¹ is phenyl and there is no R¹⁵. This compound of Formula III has the structure:

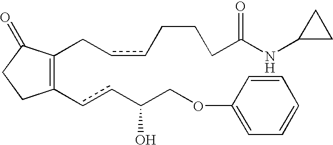

wherein a hashed line represents a substituent below the plane of this paper, and a dashed line represents a single or a double bond which can be in the cis or trans configuration. A compound where the dashed lines are double bonds with the stereoconfigurations as drawn is 16-phenoxy-17,18,19,20-tetranor PGB₂ N-cyclopropylamide. A compound where the C₅-C₆ dashed line is a single bond, and the C₁₃-C₁₄ dashed line is a double bond with the stereoconfigurations as drawn is 16-phenoxy-17,18,19,20-tetranor PGB₁ N-cyclopropylamide.

In some embodiments, R⁵ is H, D¹ is —C(O)NR⁹₂, R⁹₂ is H and a nitrogen-linked-cyclopropylmethyl group, F is —O—, G¹ is phenyl and there is no R¹⁵. This compound of Formula III has the structure:

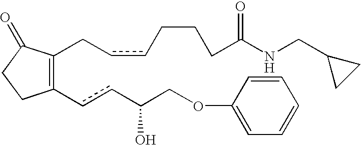

wherein a hashed line represents a substituent below the plane of this paper, and a dashed line represents a single or a double bond which can be in the cis or trans configuration. A compound, where the dashed lines are double bonds with the stereoconfigurations as drawn is 16-phenoxy-17,18,19,20-tetranor PGB₂ N-cyclopropylmethylamide. A compound where the C₅-C₆ dashed line is a single bond, and the C₁₃-C₁₄ dashed line is a double bond with the stereoconfigurations as drawn is 16-phenoxy-17,18,19,20-tetranor PGB₁ N-cyclopropylmethylamide.

In some embodiments, R⁵ is H, D¹ is —C(O)NR⁹₂, R⁹₂ is H and a nitrogen-linked-cyclopropyl group, F is —CH₂—, G¹ is phenyl and there is no R¹⁵. This compound of Formula III has the structure:

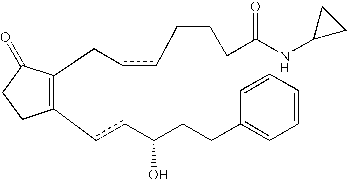

wherein a hashed line represents a substituent below the plane of this paper, and a dashed line represents a single bond or a double bond which can be in the cis or trans configuration. A compound where the dashed lines are double bonds with the stereoconfigurations as drawn is 17-phenyl-18,19,20-trinor PGB₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGB₁ N-cyclopropylamide.

In some embodiments, R⁵ is H, D¹ is —C(O)NR⁹₂, R⁹₂ is H and a nitrogen-linked-cyclopropylmethyl group, F is —CH₂—, G¹ is phenyl and there is no R¹⁵. This compound of Formula III has the structure:

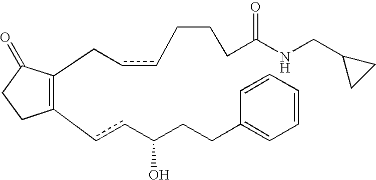

wherein a hashed line represents a substituent below the plane of this paper, and a dashed line represents a single bond or a double bond which can be in the cis or trans configuration. A compound, where the dashed lines are double bonds with the stereoconfigurations as drawn is 17-phenyl-18,19,20-trinor PGB₂ N-cyclopropylmethylamide. A compound where the C₅-C₆ dashed line is a single bond, and the C₁₃-C₁₄ dashed line is a double bond with the stereoconfigurations as drawn is 17-phenyl-18,19,20-trinor PGB₁ N-cyclopropylmethylamide.

In some embodiments, R⁵ is H, D¹ is —C(O)NR⁹₂, R⁹₂ is II and a nitrogen-linked —CH(CH₂OH)₂ group, F is —CH₂—, G^{I} is phenyl and there is no R¹⁵. This compound of Formula III has the structure:

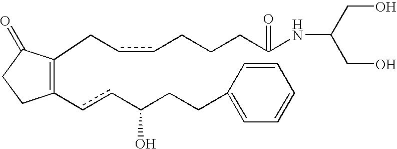

wherein a hashed line represents a substituent below the plane of this paper, and a dashed line represents a single bond or a double bond which can be in the cis or trans configuration. A compound wherein the dashed lines are double bonds with the stereoconfigurations as drawn is 17-phenyl-18,19,20-trinor PGB₂ (N-(1,3-dihydroxypropan-2-yl))amide. A compound wherein the C₅-C₆ dashed line is a single bond, and the C₁₃-C₁₄ dashed line is a double bond with the stereoconfigurations as drawn is 17-phenyl-18,19,20-trinor PGB₁ (N-(1,3-dihydroxypropan-2-yl))amide.

The following are nonlimiting embodiments depicting cyclopentane ring X of the compound of Formula III, wherein R¹ is ═O, R² is H, and R³ is H, which has the structure:

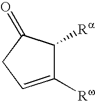

In some embodiments, R⁵ is H, D¹ is —C(O)NR⁹₂, R⁹₂ is H and a nitrogen-linked —N-linked cyclopropyl group, F is —O—, G¹ is phenyl and there is no R¹⁵. This compound of Formula III has the structure:

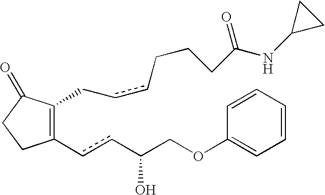

wherein a hashed line represents a substituent below the plane of this paper, and a dashed line represents a single bond or a double bond which can be in the cis or trans configuration. This compound, where the dashed lines are double bonds with the stereoconfigurations as drawn is 16-phenoxy-17,18,19,20-tetranor PGC₂ N-cyclopropylamide. A compound where the C₅-C₆ dashed line is a single bond, and the C₁₃-C₁₄ dashed line is a double bond with the stereoconfigurations as drawn is 16-phenoxy-17,18,19,20-tetranor PGC₁ N-cyclopropylamide.

In some embodiments, R⁵ is H, D¹ is —C(O)NR⁹₂, R⁹₂ is H and a nitrogen-linked-cyclopropylmethyl group, F is —O—, G¹ is phenyl and there is no R¹⁵. This compound of Formula III has the structure:

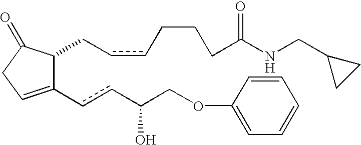

wherein a hashed line represents a substituent below the plane of this paper, and a dashed line represents a single bond or a double bond which can be in the cis or trans configuration. The compound where the dashed lines are double bonds with the stereoconfigurations as drawn is 16-phenoxy-17,18,19,20-tetranor PGC₂ N-cyclopropylmethylamide. A compound where the C₅-C₆ dashed line is a single bond, and the C₁₃-C₁₄ dashed line is a double bond with the stereoconfigurations as drawn is 16-phenoxy-17,18,19,20-tetranor PGC₁ N-cyclopropylmethylamide.

In some embodiments, R⁵ is H, D¹ is —C(O)NR⁹₂, R⁹₂ is H and a nitrogen-linked-cyclopropyl group, F is —CH₂—, G¹ is phenyl and there is no R¹⁵. This the compound of Formula III has the structure:

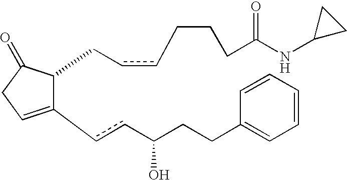

wherein a hashed line represents a substituent below the plane of this paper, and a dashed line represents a single bond or a double bond which can be in the cis or trans configuration. The compound where the dashed lines are double bonds with the stereoconfigurations as drawn is 17-phenyl-18,19,20-trinor PGC₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGC₁ N-cyclopropylamide.

In some embodiments, R⁵ is H, D¹ is —C(O)NR⁹₂, R⁹₂ is H and a nitrogen-linked-cyclopropylmethyl group, F is —CH₂—, G¹ is phenyl and there is no R¹⁵. This compound of Formula III has the structure:

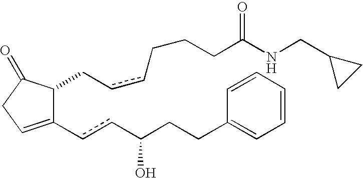

wherein a hashed line represents a substituent below the plane of this paper, and a dashed line represents a single bond or a double bond which can be in the cis or trans configuration. The compound, where the dashed lines are double bonds with the stereoconfigurations as drawn is 17-phenyl-18,19,20-trinor PGC₂ N-cyclopropylmethyl amide. A compound where the C₅-C₆ dashed line is a single bond, and the C₁₃-C₁₄ dashed line is a double bond with the stereoconfigurations as drawn is 17-phenyl-18,19,20-trinor PGC_{I} N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G^{I} is phenyl and there is no R¹⁵. This compound of Formula III has the structure:

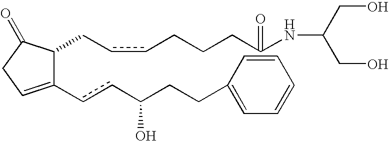

wherein a hashed line represents a substituent below the plane of this paper, and a dashed line represents a single bond or a double bond which can be in the cis or trans configuration. A compound wherein the dashed lines are double bonds with the stereoconfigurations as drawn is 17-phenyl-18,19,20-trinor PGC₂ (N-(1,3-dihydroxypropan-2-yl))amide. A compound wherein the C₅-C₆ dashed line is a single bond, and the C₁₃-C₁₄ dashed line is a double bond with the stereoconfigurations as drawn is 17-phenyl-18,19,20-trinor PGC₁ (N-(1,3-dihydroxypropan-2-yl))amide

The following are nonlimiting embodiments depicting cyclopentane ring X of the compound of Formula III, wherein R¹ is OH, and R³ is ═O, which has the structure:

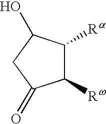

In some embodiments, R⁵ is H, D¹ is —C(O)NR⁹₂, R⁹₂ is H and a nitrogen-linked-cyclopropyl group, F is —O—, G¹ is phenyl and there is no R¹⁵. This compound of Formula III has the structure:

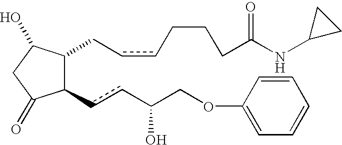

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 16-phenoxy-17,18,19,20-tetranor PGD₂ N-cyclopropylamide. A compound where the C₅-C₆ dashed line is a single bond, and the C₁₃-C₁₄ dashed line is a double bond with the stereoconfigurations as drawn is 16-phenoxy-17,18,19,20-tetranor PGD₁ N-cyclopropylamide.

In some embodiments, R⁵ is H, D¹ is —C(O)NR⁹₂, R⁹₂ is H and a nitrogen-linked cyclopropylmethyl group, F is —O—, G¹ is phenyl and there is no R¹⁵. This compound of Formula I has the structure:

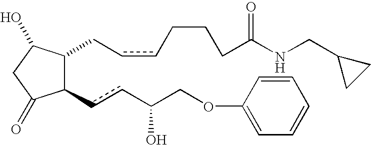

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 16-phenoxy-17,18,19,20-tetranor PGD₂ N-cyclopropylmethylamide. A compound where the C₅-C₆ dashed line is a single bond, and the C₁₃-C₁₄ dashed line is a double bond with the stereoconfigurations as drawn is 16-phenoxy-17,18,19,20-tetranor PGD₁ N-cyclopropylmethylamide.

In some embodiments, R⁵ is H, D¹ is —C(O)NR⁹₂, R⁹₂ is H and a nitrogen-linked cyclopropyl group, F is —CH₂—, G^{I} is phenyl and there is no R¹⁵. This compound of Formula I has the structure:

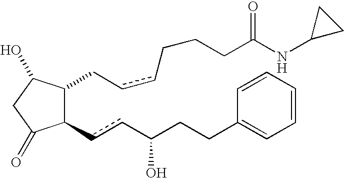

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 17-phenyl-18,19,20-trinor PGD₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGD₁ N-cyclopropylamide.

In some embodiments, R⁵ is H, D¹ is —C(O)NR⁹₂, R⁹₂ is H and a nitrogen-linked cyclopropylmethyl group, F is —CH₂—, G' is phenyl and there is no R¹⁵. This compound of Formula I has the structure:

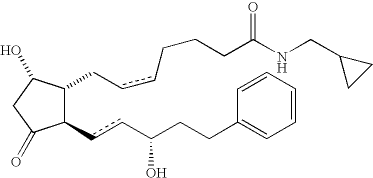

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfigurations as drawn is 17-phenyl-18,19,20-trinor PGD₂ N-cyclopropylmethylamide. A compound where the C₅-C₆ dashed line is a single bond, and the C₁₃-C₁₄ dashed line is a double bond with the stereoconfigurations as drawn is 17-phenyl-18,19,20-trinor PGD₁ N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R'⁵. This compound of Formula III has the structure:

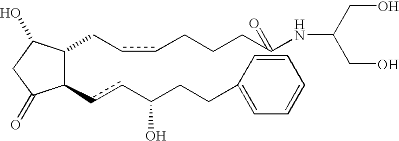

wherein a hashed line represents a substituent below the plane of this paper, a bold wedge represents a substituent above the plane of this paper, and a dashed line represents a single bond or a double bond which can be in the cis or trans configuration. A compound wherein the dashed lines are double bonds with the stereoconfigurations as drawn is 17-phenyl-18,19,20-trinor PGD₂ (N-(1,3-dihydroxypropan-2-yl))amide. A compound wherein the C₅-C₆ dashed line is a single bond, and the C₁₃-C₁₄ dashed line is a double bond with the stereoconfigurations as drawn is 17-phenyl-18,19,20-trinor PGD₁ (N-(1,3-dihydroxypropan-2-yl))amide.

The following are nonlimiting embodiments depicting cyclopentane ring X of the compound of Formula III, wherein R¹ is ═O, and R³ is OH or OR⁴, which has the structure:

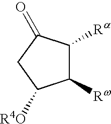

In one embodiment, R⁴ and R⁵ are both H, D¹ is —C(O)NR⁹₂, R⁹₂ is H and a nitrogen-linked cyclopropyl group, F is —CH₂—, G' is phenyl and there is no R¹⁵. This compound of Formula III has the structure:

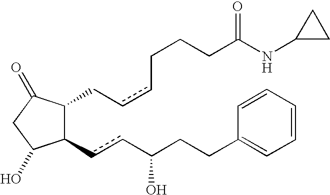

wherein a hashed line represents a substituent below the plane of this paper, a bold wedge represents a substituent above the plane of this paper, and a dashed line represents a single bond or a double bond which can be in the cis or trans configuration. A compound where the dashes represent double bonds, with the stereoconfigurations as drawn is 17-phenyl-18,19,20-trinor PGE₂ N-cyclopropylamide. A compound wherein the C₅-C₆ dashed line is a single bond, and the C₁₃-C₁₄ dashed line is a double bond with the stereoconfigurations as drawn is 17-phenyl-18,19,20-trinor PGE₁ N-cyclopropylamide.

In another embodiment, R⁴ and R⁵ are both H, D^{I} is —C(O)NR⁹₂R⁹₂ is H and a nitrogen-linked cyclopropylmethyl group, F is —CH₂—, G¹ is phenyl and there is no R¹⁵. This compound of Formula III has the structure:

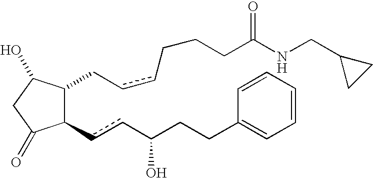

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfigurations as drawn, is 17-phenyl-18,19,20-trinor PGE₂ N-cyclopropylmethylamide. A compound wherein the C₅-C₆ dashed line is a single bond, and the C₁₃-C₁₄ dashed line is a double bond with the stereoconfigurations as drawn is 17-phenyl-18,19,20-trinor PGE₁ N-cyclopropylmethylamide.

In yet another embodiment, R⁴ is —PO₃⁻² with pharmaceutically acceptable counter ion(s) such as 2 Na⁺, R⁵ is H, F is O, G^{I} is phenyl, there is no R¹⁵, D¹ is —C(O)OR⁸, and R⁸ is a cyclopropyl group. This compound of Formula III has the following structure (counterions not shown):

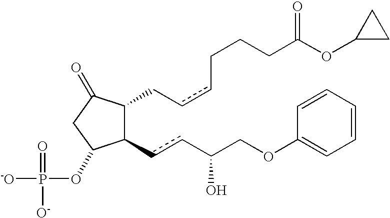

wherein a hashed line represents a substituent below the plane of this paper, a bold wedge represents a substituent above the plane of this paper, and a dashed line represents a single bond or a double bond which can be in the cis or trans configuration.

The following are nonlimiting embodiments depicting cyclopentane ring X of the compound of Formula III, wherein R¹ is OH or OR⁴, and R³ is OH or OR⁴, which has the structure:

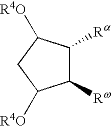

In one embodiment, R⁵ is H, D¹ is —C(O)NR⁹₂, R⁹₂ is H and a nitrogen-linked cyclopropyl group, F is —CH₂, G^{I} is phenyl, and there is no R¹⁵ group. This compound of Formula III, has the structure:

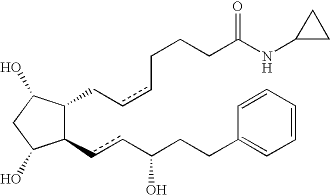

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn is 17-phenyl-18,19,20-trinor PGF_{2α} N-cyclopropylamide. A compound wherein the C₅-C₆ dashed line is a single bond and the C₁₃-C₁₄ dashed line is a double bond with the stereoconfigurations as drawn is 17-phenyl-18,19,20-trinor PGF_{2α} N-cyclopropylamide.

In another embodiment, R⁵ is H, D¹ is —C(O)NR⁹₂, R⁹₂ is H and a nitrogen-linked cyclopropylmethyl group, F is —CH₂, G¹ is phenyl, and there is no R¹⁵. This compound of Formula III has the structure:

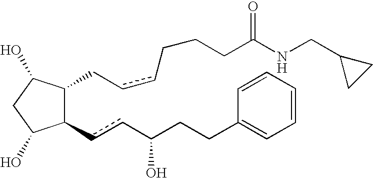

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 17-phenyl-18,19,20-trinor PGF₂, N-cyclopropylmethylamide. A compound wherein the C₅-C₆ dashed line is a single bond and the C₁₃-C₁₄ dashed line is a double bond with the stereoconfigurations as drawn is 17-phenyl-18,19,20-trinor PGF_{2α} N-cyclopropylmethylamide.

In yet another embodiment, R¹ is OR⁴, where R⁴ is PO₃²⁻ with pharmaceutically acceptable counterion(s) such as 2 Na⁺, D¹ is —C(O)OR⁸, R⁵ is H, F is —O—, G¹ is phenyl, there is no R¹⁵, and R⁸₂ is a cyclopropyl group. This compound of Formula III has the structure:

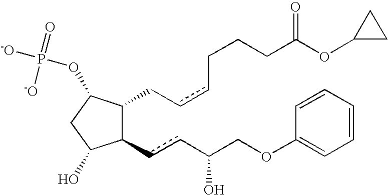

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane.

In another embodiment, R⁵ is H, D^{I} is —C(O)NR⁹₂, R⁹₂ is H and a nitrogen-linked cyclopropyl group, F is —O—, G¹ is phenyl, there is no R¹⁵. This compound of Formula III has the structure:

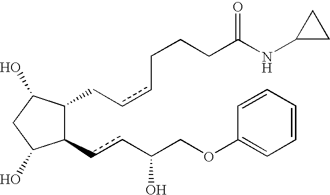

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. This compound, where the dashed lines are double bonds with the stereoconfigurations as drawn, is 16-phenoxy-17,18,19,20-tetranor PGF_{2α} N-cyclopropylamide. A compound wherein the C₅-C₆ dashed line is a single bond and the C₁₃-C₁₄ dashed line is a double bond with the stereoconfigurations as drawn is 16-phenoxy-17,18,19,20-tetranor PGF_{2G}, N-cyclopropylamide.

In another embodiment, R⁵ is H, D^{E} is —C(O)NR⁹₂, R⁹₂ is H and a nitrogen-linked cyclopropylmethyl group, F is —O—, G¹ is phenyl, and there is no R¹⁵. This compound of Formula III has the structure:

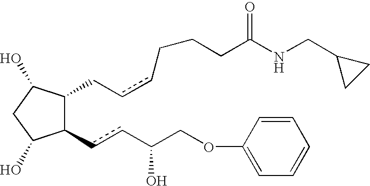

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 16-phenoxy-17,18,19,20-tetranor PGF_{2α} N-cyclopropylmethylamide. A compound wherein the C₅-C₆ dashed line is a single bond and the C₁₃-C₁₄ dashed line is a double bond with the stereoconfigurations as drawn is 16-phenoxy-17,18,19,20-tetranor PGF_{2α}N-cyclopropylmethylamide.

In another embodiment, R⁵ is H, D¹ is —C(O)NR⁹₂, R⁹₂ is H and a nitrogen-linked cyclopropyl group, F is —CH₂, G¹ is pyrimidin-2-yl, and there is no R¹⁵. This compound of Formula III has the structure:

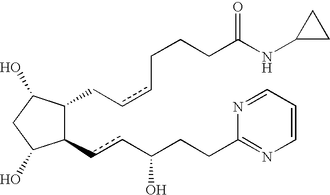

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 17-pyrimidin-2-yl-18,19,20-trinor PGF₂ N-cyclopropylamide. A compound wherein the C₅-C₆ dashed line is a single bond and the C₁₃-C₁₄ dashed line is a double bond with the stereoconfigurations as drawn is 17-pyrimidin-2-yl-18,19,20-trinor PGF_{2α} N-cyclopropylamide.

In another embodiment, R¹ is OR⁴ where R⁴ is —(PO₂(OH))ₛH, where s is 1˜25 or a pharmaceutically acceptable salt thereof, R⁵ is H, F is S(O), G^{I} is phenyl, D^{I} is —C(O)OR⁸, and R⁸ is a cyclopropyl group. This compound of Formula III has the structure:

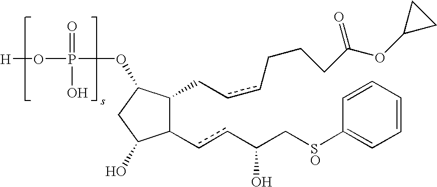

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound, when the dashed lines are double bonds with the stereoconfigurations as drawn, is a poly-hydroxyphosphoryloxy derivative of cyclopropyl 7-((1R,3R,5S)-3-hydroxy-2-((3R,E)-3-hydroxy-4-(phenylsulfinyl)but-1-enyl)-5-(phosphonooxy)cyclopentyl)heptanoate. A compound wherein the C₅-C₆ dashed line is a single bond and the C₁₃-C₁₄ dashed line is a double bond with the stereoconfigurations as drawn is cyclopropyl 7-((1R,3R,5S)-3-hydroxy-5-phosphonooxy-2-((3R,E)-3-hydroxy-4-(phenylsulfinyl)but-1-enyl)cyclopentyl)heptanoate.

In yet another embodiment, R¹ is OR⁴ and R⁴ is a monosaccharide (e.g., glucose), R⁵ is H, D¹ is —C(O)NR⁹₂, R⁹₂ is H and a nitrogen-linked ethyl group; F is —CH₂—, G¹ is phenyl, and there is no R¹⁵. This compound of Formula III has the structure:

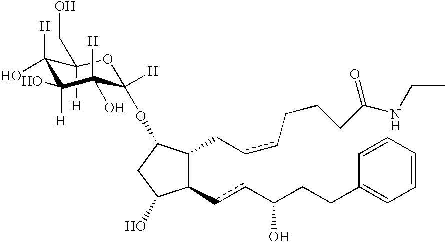

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane.

In yet another embodiment, R¹ is OR⁴ and R⁴ is a monosaccharide (e.g., glucose), R⁵ is H, D¹ is —C(O)NR⁹₂, R⁹₂ is H and a nitrogen-linked ethyl group; F is —CH₂—, G¹ is 1,3-oxazinan-2-yl and R¹⁵ is a cyclopropyl group. This compound of Formula III has the structure:

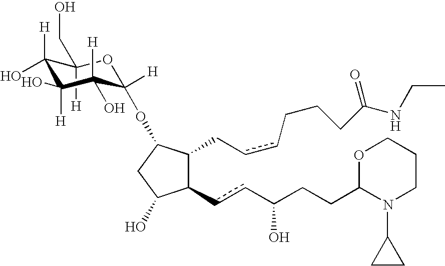

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane.

In another embodiment, R⁵ is H, D¹ is —C(O)NR⁹₂, R⁹₂ is H and cyclopropyl; F is —O—; G¹ is phenyl and R¹⁵ is Cl. This compound of Formula III has the structure:

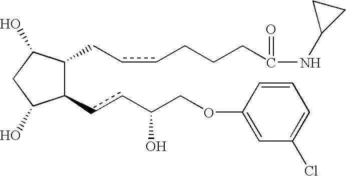

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound, where the dashed lines are double bonds, is 16-(3-chlorophenoxy)-17,18,19,20-tetranor PGF2α N-cyclopropylamide. A compound wherein the C₅-C₆ dashed line is a single bond and the C₁₃-C₁₄ dashed line is a double bond with the stereoconfigurations as drawn is 16-(3-chlorophenoxy)-17,18,19,20-tetranor PGF_{2α} N-cyclopropylamide.

In another embodiment, R⁵ is H, D¹ is —C(O)NR⁹₂, R⁹₂ is H and N-cyclopropylmethylamide; F is —O—; G¹ is phenyl; and R¹⁵ is Cl. This compound of Formula III has the structure:

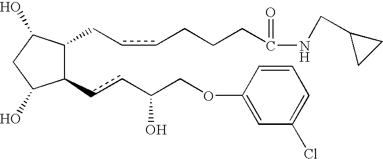

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound, where the dashed lines are double bonds with the stereoconfigurations as drawn, is 16-(3-chlorophenoxy)-17,18,19,20-tetranor PGF2α-N-cyclopropylmethylamide. A compound wherein the C₅-C₆ dashed line is a single bond and the C₁₃-C₁₄ dashed line is a double bond with the stereoconfigurations as drawn is 16-(3-chlorophenoxy)-17,18,19,20-tetranor PGF₂, N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G^{I} is phenyl and there is no R¹⁵. This compound of Formula III has the structure:

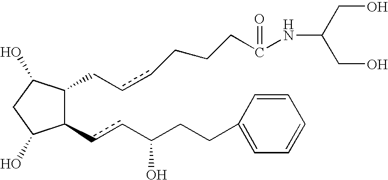

wherein a hashed line represents a substituent below the plane of this paper, a bold wedge represents a substituent above the plane of this paper, and a dashed line represents a single bond or a double bond having the configuration indicated. A compound wherein the dashed lines are double bonds with the stereoconfigurations as drawn is 17-phenyl-18,19,20-trinor PGF2α (N-(1,3-dihydroxypropan-2-yl))amide. A compound wherein the C₅-C₆ dashed line is a single bond and the C₁₃-C₁₄ dashed line is a double bond with the stereoconfigurations as drawn is 17-phenyl-18,19,20-trinor PGF_{2α} (N-(1,3-dihydroxypropan-2-yl))amide.

The following are nonlimiting embodiments depicting cyclopentane ring X of the compound of Formula III, wherein R¹ is H, R² is H, and R³ is ═O, which has the structure:

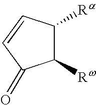

In some embodiments, R⁵ is H, D¹ is —C(O)NR⁹₂, R⁹₂ is H and a nitrogen-linked-cyclopropyl group, F is —O—, G¹ is phenyl and there is no R¹⁵. This compound of Formula III has the structure:

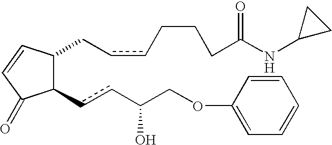

wherein a hashed line represents a substituent below the plane of this paper, a bold wedge represents a substituent above the plane of this paper, and a dashed line represents a single or a double bond. A compound where the dashed lines are double bonds with the stereoconfigurations as drawn is 16-phenoxy-17,18,19,20-tetranor PGJ₂ N-cyclopropylamide. A compound wherein the C₅-C₆ dashed line is a single bond and the C₁₃-C₁₄ dashed line is a double bond with the stereoconfigurations as drawn is 16-phenoxy-17,18,19,20-tetranor PGJ₁ N-cyclopropylamide.

In some embodiments, R⁵ is H, D¹ is —C(O)NR⁹₂, R⁹₂ is H and a nitrogen-linked-cyclopropylmethyl group, F is —O—, G¹ is phenyl and there is no R¹⁵. This compound of Formula III has the structure:

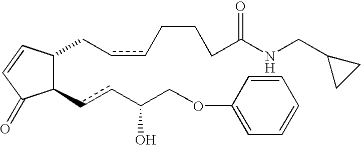

wherein a hashed line represents a substituent below the plane of this paper, a bold wedge represents a substituent above the plane of this paper, and a dashed line represents a single or a double bond having the configuration indicated. A compound where the dashed lines are double bonds with the stereoconfigurations as drawn is 16-phenoxy-17,18,19,20-tetranor PGJ₂ N-cyclopropylmethylamide. A compound wherein the C₅-C₆ dashed line is a single bond and the C₁₃-C₁₄ dashed line is a double bond with the stereoconfigurations as drawn is 16-phenoxy 17,18,19,20-tetranor PGJ₁ N-cyclopropylmethylamide.

In some embodiments, R⁵ is H, D¹ is —C(O)NR⁹₂, R⁹₂ is H and a nitrogen-linked-cyclopropyl group, F is —CH₂—, G¹ is phenyl and there is no R¹⁵. This compound of Formula III has the structure:

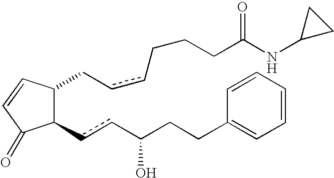

wherein a hashed line represents a substituent below the plane of this paper, a bold wedge represents a substituent above the plane of this paper, and a dashed line represents a single or a double bond. A compound, where the dashed lines are double bonds with the stereoconfigurations as drawn is 17-phenyl-18,19,20-trinor PGD₂ N-cyclopropylamide. A compound wherein the C₅-C₆ dashed line is a single bond and the C₁₃-C₁₄ dashed line is a double bond with the stereoconfigurations as drawn is 17-phenyl-18,19,20-trinor PGJ₁ N-cyclopropylamide.

In some embodiments, R⁵ is H, D¹ is —C(O)NR⁹₂, R⁹₂ is H and a nitrogen-linked-cyclopropylmethyl group, F is —CH₂—, G¹ is phenyl and there is no R¹⁵. This compound of Formula III has the structure:

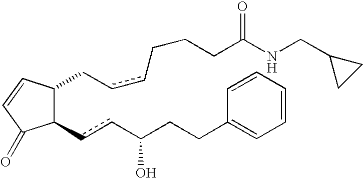

wherein a hashed line represents a substituent below the plane of this paper, a bold wedge represents a substituent above the plane of this paper, and a dashed line represents a single bond or a double bond. A compound where the dashed lines are double bonds with the stereoconfigurations as drawn is 17-phenyl-18,19,20-trinor PGJ₂ N-cyclopropylmethylamide. A compound wherein the C₅-C₆ dashed line is a single bond and the C₁₃-C₁₄ dashed line is a double bond with the stereoconfigurations as drawn is 17-phenyl-18,19,20-trinor PGJ₁ N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula III has the structure:

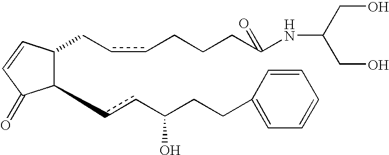

wherein a hashed line represents a substituent below the plane of this paper, a bold wedge represents a substituent above the plane of this paper, and a dashed line represents a single bond or a double bond. A compound wherein the dashed lines are double bonds with the stereoconfigurations as drawn is 17-phenyl-18,19,20-trinor PGJ₂ (N-(1,3-dihydroxypropan-2-yl)) amide. A compound wherein the C₅-C₆ dashed line is a single bond and the C₁₃-C₁₄ dashed line is a double bond with the stereoconfigurations as drawn is 17-phenyl-18,19,20-trinor PO_{I} (N-(1,3-dihydroxypropan-2-yl)) amide.

According to another embodiment, the at least one compound of Formula III is diastereomerically pure. According to another embodiment, the at least one compound of Formula III may be a mixture of diastereomers in any ratio. According to another embodiment, the at least one compound of Formula III may be enantiomerically pure. According to another embodiment, the at least one compound of Formula III may be a mixture of enantiomers in any ratio, including a racemate. According to another embodiment, the at least one compound of Formula III may be diastereomerically and enantiomerically pure. According to another embodiment, the at least one compound of Formula III may be a mixture of diastereomers and enantiomers in any ratio. According to another embodiment, the at least one compound of Formula III may have one or more hydrogen atoms replaced by deuterium.

According to some embodiments, the compound of Formula III is latanoprost. According to another embodiment, the compound of Formula III is travoprost. According to another embodiment, the compound of Formula III is travoprost ethylamide. According to another embodiment, the compound of Formula III is bimatoprost. According to another embodiment, the compound of Formula III is fluprostenol. According to another embodiment, the compound of Formula III is fluprostenol isopropyl ester. According to another embodiment, the compound of Formula III is fluprostenol methyl amide. According to another embodiment, the compound of Formula III is 9-keto fluprostenol isopropyl ester. According to another embodiment, the compound of Formula III is cloprostenol. According to another embodiment, the compound of Formula III is cloprostenol isopropyl ester. According to another embodiment, the compound of Formula III is chloprostenol methyl amide. According to another embodiment, the compound of Formula III is 17 phenyl-18,19,20-trinor prostaglandin E2 (N-(1,3-dihydroxypropan-2-yl))amide. According to another embodiment, the compound of Formula III is 17 phenyl-18,19,20-trinor prostaglandin F2α (N-(1,3-dihydroxypropan-2-yl))amide.

Imidazole Analogs

The term “imidazole analog” is used herein to describe an imidazole or related 5-membered aromatic heterocycle that contains at least two nitrogen atoms in the ring according to [Formula IV], or a hydrate, solvate, salt, zwitterion, N-oxide or tautomer thereof:

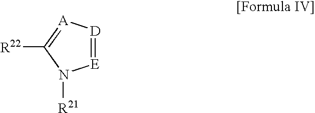

wherein A is N; —NR²⁰; —NR⁶³ or CH;

wherein D is N or CR²³;

wherein E is N; —NR⁶³ or CR²⁴;

with the proviso that, if A is —CH, then at least one among D and E is a nitrogen-containing moiety;

with the further proviso that A and E are not simultaneously both NR⁶³;

wherein R²³ is H; F, Cl, Br; I; —NO₂; —N(R⁴²)₂; straight chain or branched C₁˜C₆ alkyl, alkenyl or alkoxy; or phenyl;

wherein R²⁴ is H; F, Cl, Br; I; —NO₂; —N(R⁴₂)₂; straight chain or branched C₁˜C₆ alkyl, alkenyl or alkoxy; or phenyl;

or wherein R²³ and R²⁴ together are —CR⁶²═CR⁶²—CR⁶²═CR⁶²—;

wherein R²⁰ is selected from the group consisting of: H; straight chain or branched C₁˜C₁₂ alkyl;

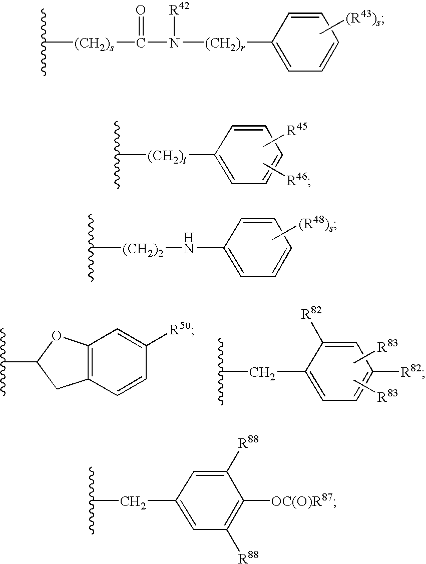

—(CH₂)ₜC(O)R⁴⁴; —CH(R⁴⁷)₂; —(CH₂)ᵤOR⁴⁹; —(CH²)ᵤSR⁴⁹; —CH₂CH(OH)R⁵¹; —CH₂CH═CHR⁵²; or —CH₂C(O)CH₂OR⁵²; and the compound of Formula IV is an imidazolium or triazolium salt with a pharmaceutically acceptable counter anion or an internal salt (zwitterion); or

wherein R⁶³ is a moiety that is readily cleaved in vivo, exemplified by, but not limited to, —CHR⁴²OC(═O)(O)ₙR⁵⁵ or —C(R⁴²)₂OP(═O)(OR¹⁰⁶)₂ and the compound of Formula IV is a prodrug and an imidazolium or triazolium salt with a pharmaceutically acceptable counter ion or an internal salt (zwitterion);

wherein s is 1 or 2;

wherein t is an integer from 1 to 4 inclusive;

wherein u is 2 or 3;

wherein R²² is H; F, Cl, Br; I; —NO₂; —N(R⁴²)₂; straight chain or branched C₁˜C₆ alkyl, alkoxy, alkenyl or hydroxyalkyl; phenyl; or a 5˜6-membered aromatic heterocyclic radical containing one or more N, O or S atoms, said heterocyclic radical preferably containing one S and one or two N atoms, said heterocyclic radical being preferably thiazolyl and more preferably thiazol-4-yl;

wherein R²¹ is H; or —(CR²⁵R²⁶)ₘ—CR²⁷R²⁸-Q-R²⁹;

wherein each m is independently an integer from 0 to 4 inclusive;

wherein R²⁵ is H; —C≡N; straight chain or branched C₁˜C₁₂ acyclic hydrocarbon group, said hydrocarbon group optionally containing one or more C═C or C≡C bonds; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;

wherein R²⁶ is H; or straight chain or branched C₁˜C₆ alkyl;

wherein R²⁷ is -T-U—V; —O—(CH₂)ᵣ-L; —NH—(CH₂)ᵣ-L; —S(O)ᵣ—R⁵⁵; —OR⁵³; —OR⁶⁵; —CF(R⁶⁷)R⁵⁶; —CF(R⁶⁷)C(O)R⁶⁸; —CF(R⁶⁷)C(O)N(R⁶⁸)₂; —CF(R⁶⁷)C(O)N(R⁶⁸)N(R⁶⁸)₂; —CF(R⁶⁷)C(O)N(R⁶⁸)OR⁶⁸; —CF(R⁶⁷)C(O)N(R⁶⁸)OH; —CF(R⁶⁷)C(═NH)R⁶⁸; —CF(R⁶⁷)C(═NH)N(R⁶⁸)₂; —CF(R⁶⁷)C(═NH)N(R⁶⁸)N(R⁶⁸)₂; —CF(R⁶⁷)C(NH)N(R⁶⁸)OR⁶⁸; —CF(R⁶⁷)C(═NH)N(R⁶⁸)OH; or —CF(R⁶⁷)C(═N—OR⁶⁹)NH₂;

wherein each r is independently an integer from 0 to 2 inclusive;

wherein each L is independently α-tetralyl; or phenyl, said phenyl being optionally substituted with up to three groups selected from the group consisting of: straight chain or branched C₁˜C₆ alkyl, straight chain or branched C₁˜C₆ alkoxy, —C≡N, —NO₂, or —NH₂;

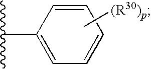

wherein R²⁸ is H; F; Cl; Br; —OR⁴²; —OC(═O)R¹⁰⁰; straight chain or branched C₁˜C₆ alkyl; R61; or —OP(═O)(OH)₂ or a pharmaceutically acceptable salt or zwitterion form thereof;

wherein each Q is independently a covalent bond; —O—; —S—; —S(O)—; or —S(O)₂—;

wherein R²⁹ is C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R³⁰ groups; straight chain or branched C₁˜C₆ alkyl optionally substituted with CO₂H; or

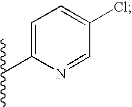

wherein each p is independently an integer from 0 to 3 inclusive;

wherein each T is independently a covalent bond; —CH₂CH₂—; —CH═CH—; —(O)ₙC(R⁴²)₂—; —CH₂O—; —SCH₂—; —CH₂S—; —OCH₂CH₂O—; —CH(CH₃)—; —CH₂—; —CF₂—; —C(R⁹³)₂—; or

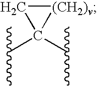

wherein each V is independently an integer from 1 to 3 inclusive;

wherein each U is independently a covalent bond;

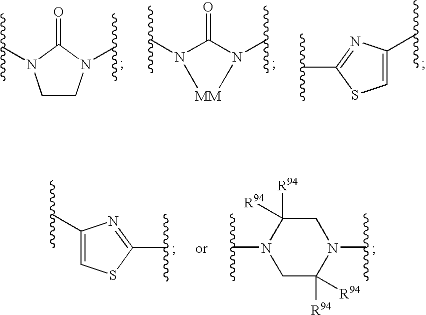

wherein each V is independently H; —S(O)₂CH₃; —C(O)NH₂; —CH₂C≡CH; —CH═CH₂; —S(O)ᵣ—R⁵⁵;

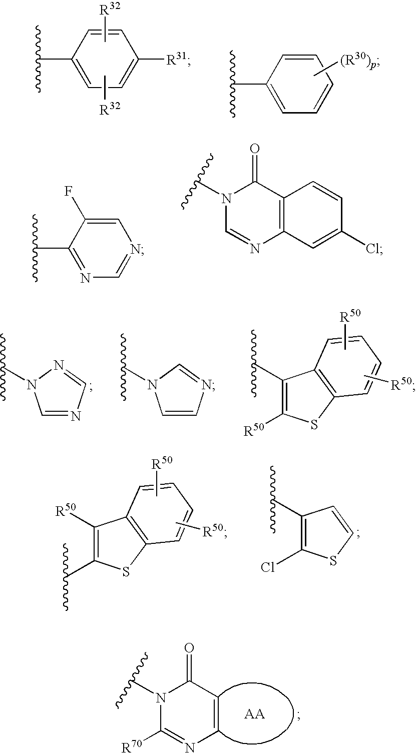

### —R⁵⁹; —R⁶¹; or -LL-R⁹⁵;

wherein AA represents a benzene ring or a 5- or 6-membered heterocyclic ring wherein one or more of the ring atoms are selected from the group consisting of N, O and S, which rings can be optionally fused to a benzene ring or to a 5- or 6-membered heterocyclic ring containing one or more heteroatoms selected from N, O and S and wherein AA can be unsubstituted or have 1, 2, 3 or 4 substituents R⁷¹ in any of the rings;

wherein each R³⁰ is independently F; Cl; Br; I; —NO₂; —C≡N; —S—C≡N; —NR³⁹R⁴⁰; —OR⁴¹; —OR⁵⁴; —OH; —OC(O)R⁵⁴; —C(O)R⁵⁴; —C(O)OR⁵⁴; —C(O)OH; —N(R⁵⁴)C(O)OR⁵⁴; —OC(O)N(R⁵⁴)₂; —SH; —SR⁴¹; —S(O)ᵣR⁵⁸; —CH═CH—CO₂H;

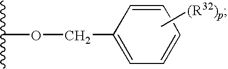

straight chain or branched C₁˜C₁₂ alkyl; straight chain or branched C₁˜C₁₀ alkoxy; methylenedioxy; straight chain or branched C₁˜C₆ cyanoalkyl exemplified by, but not limited to CH₂C≡N, —CH₂CH₂C≡N, —CH₂CH₂CH₂C≡N, and —CH₂CH(CH₃)C≡N; straight chain or branched C₁˜C₆ halogenoalkyl exemplified by, but not limited to —CF₃, —CF₂CF₃, —CH₂CF₃, —CHF₂, —CH₂F, —CH₂Cl, and —CH₂Br; straight chain or branched C₁˜C₅ halogenoalkoxy exemplified by, but not limited to —OCF₃, —OCF₂CF₃, —OCH₂CF₃, —OCHF₂, and —OCH₂F; straight chain or branched C₁˜C₆ alkylthio or haloalkylthio; straight chain or branched C₁˜C₆ alkylthionyl or haloalkylthionyl; or straight chain or branched C₁˜C₆ alkylsulfonyl or haloalkylsulfonyl; C₃˜C₇ cycloalkyl, C₆˜C₁₄ aryl containing one, two or three rings, or C₆˜C₁₄ aryloxy containing one, two or three rings, said cycloalkyl, aryl or aryloxy being optionally substituted with one to three moieties independently selected from F, Cl, Br, I, —NO₂, or straight chain or branched C₁˜C₆ alkyl, halogenoalkyl or alkoxy; straight chain or branched C₁˜C₄ alkyl substituted with C₃˜C₈ cycloalkyl or C₆˜C₁₄ aryl containing one, two or three rings, said cycloalkylalkyl or arylalkyl being optionally substituted with one to three moieties independently selected from F, Cl, Br, I, —NO₂, or straight chain or branched C₁˜C₆ alkyl, halogenoalkyl or alkoxy;

wherein each R³¹ is independently H; F; Cl; Br; I; —C≡N; —NO₂; —CF₃; —N═C═S; —N(R⁴²)₂; —NH—C(═O)-(M)ₙR⁵⁴; —NH—C(═S)—NH—R⁵⁴; straight chain or branched C₁˜C₆ alkoxy or alkylthio; straight chain or branched C₁˜C₁₂ alkyl; —(CH₂)ᵣ—R⁹⁹;

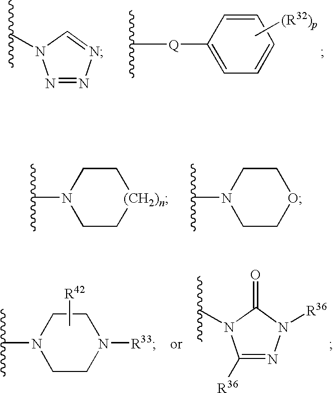

wherein each M is independently O or NH, with the proviso that when M is O and n is 1, R⁵⁴ is other than H;

wherein each R³² is independently H; F; Cl; Br; I; —C≡N; —NO₂; straight chain or branched C₁˜C₁₂ alkyl; straight chain or branched C₁˜C₄ alkoxy; straight chain or branched C₁˜C₄ halogenoalkyl exemplified by, but not limited to —CF₃, —CF₂CF₃, —CH₂CF₃, —CHF₂, —CH₂F, —CH₂Cl, —CH₂Br; straight chain or branched C₁˜C₃ halogenoalkoxy exemplified by, but not limited to —OCF₃, —OCF₂CF₃, —OCH₂CF₃, —OCHF₂, —OCH₂F; straight chain or branched C₁˜C₄ alkylthio or haloalkylthio; straight chain or branched C₁˜C_{I} alkylthionyl or haloalkylthionyl; or straight chain or branched C₁˜C₄ alkylsulfonyl or haloalkylsulfonyl;

wherein each R³³ is independently H; straight chain or branched C₁˜C₆ alkyl, wherein said alkyl is optionally substituted with —OH or —OR³⁴; —S(O)₂R³⁴; —S(O)₂—CH₂-phenyl; —C(O)R⁴²; —(CH₂)C(O)OR³⁴; —C(O)O-phenyl; —(CH₂)ₙC(O)N(R⁴²)₂; —CH₂C(S)N(R⁴²)₂; —CH₂C(S)SR³⁴; —(CH₂)n-phenyl; benzoyl, wherein said benzoyl is optionally substituted with one or two R⁴⁶ groups; or

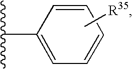

or, preferably,

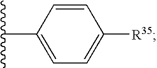

wherein each R³⁴ is independently straight chain or branched C₁˜C₆ alkyl;

wherein each R³⁵ is independently H;

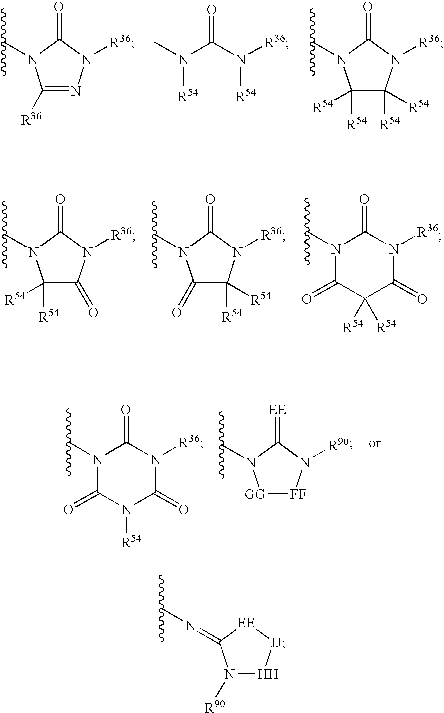

wherein each R³⁶ is independently H; straight chain or branched C₃˜C₇ alkenyl, with the proviso that the olefinic double bond is not located a to N; straight chain or branched C₃˜C₇ alkynyl, with the proviso that the alkynyl C≡C bond is not located a to N; C₃˜C₁₀ cycloalkyl, said cycloalkyl being optionally substituted with one to three R³⁰ groups; C₆˜C₁₄ aryl containing one, two or three rings, said aryl being optionally substituted with one to three R³⁰ groups; straight chain or branched C₁˜C₈ alkyl, said alkyl being optionally substituted with one to three R³⁰ groups; or

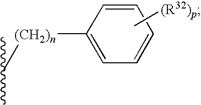

wherein each n is independently 0 or 1;

wherein R²⁶ and R²⁸ together with the two carbon atoms to which they attach may be C═C;

wherein R²⁷ and R²⁸ together may be

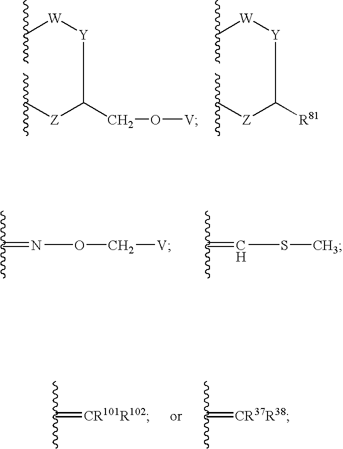

wherein W is —CH₂— and Y is —CH₂—; W is —O— and Y is —CH₂—; or W is —CH₂— and Y is —O—;

wherein Z is —CH₂— or —O—;

wherein R²⁷ together with -Q-R²⁹ may be

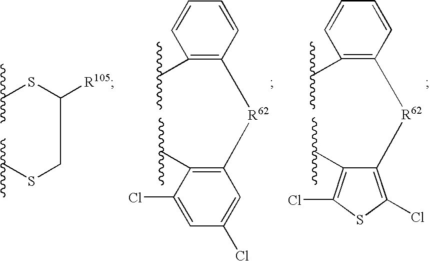

or, when R²⁷ and R²⁹ each independently represents a cycloalkyl, aryl or heterocyclic ring and Q is a covalent bond, R²⁷ together with -Q-R²⁹ may be

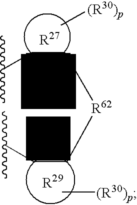

wherein each R³⁷ is independently H; C₁˜C₂ alkyl or phenyl;

wherein each R³⁸ is independently H or C₁˜C₂ alkyl;

wherein each R³⁹ is independently H; straight chain or branched C₁˜C₅ alkyl; straight chain or branched C₁˜C₅ alkanoyl; or straight chain or branched C₁˜C₅ haloalkanoyl exemplified by, but not limited to —C(O)CF₃, —C(O)CF₂CF₃, —C(O)CH₂CF₃, —C(O)CHF₂, and —C(O)CH₂F;

wherein each R⁴⁰ is independently H; straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)_{q}-G-J;

wherein each R⁴¹ is independently straight chain or branched C₁˜Cₛ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J;

wherein each q is independently an integer from 0 to 4 inclusive;

wherein each G is independently a covalent bond; —O—; or —S—;

wherein each J is independently phenyl, 2-thienyl or 3-thienyl wherein said phenyl, 2-thienyl or 3-thienyl is optionally substituted with one to three groups selected from the group consisting of F; Cl; Br; I; —NO₂; straight chain or branched C₁˜C₅ alkyl; straight chain or branched C₁˜C₅ alkoxy;

wherein each R⁴² is independently H; or straight chain or branched C₁˜C₆ alkyl;

wherein each R⁴³ is independently H; F; Cl; Br; I; —NO₂; straight chain or branched C₁˜C₆ alkyl; straight chain or branched C₁˜C₆ alkoxy; —CF₃; or —O-phenyl;

wherein each R⁴⁴ is independently 2-thienyl or 3-thienyl, wherein said 2-thienyl or 3-thienyl is optionally substituted with F, Cl, Br, or I; or phenyl, wherein said phenyl is optionally substituted with one to two identical or different groups selected from the group consisting of: F, Cl, Br, I, straight chain or branched C₁˜C₆ alkyl, or straight chain or branched C₁˜C₆ alkoxy;

wherein each R⁴⁵ is independently H; F; Cl; Br; I; —NO₂; straight chain or branched C₁˜C₆ alkyl; straight chain or branched C₁˜C₆ alkoxy; or —S(O)₂ NH₂;

wherein each R⁴⁶ is independently H; F; Cl; Br; I; straight chain or branched C₁˜C₆ alkyl; straight chain or branched C₁˜C₆ alkoxy; or —C≡N;

wherein each R⁴⁷ is independently a phenyl group optionally substituted with one to three F, Cl, Br or I;

wherein each R⁴⁸ is independently H; F; Cl; Br; I; or straight chain or branched C₁˜C₆ alkyl;

wherein each R⁴⁹ is independently 1-naphthyl; 2-naphthyl; or phenyl, wherein said phenyl is optionally substituted with one or two groups, each independently selected from the group consisting of: F, Cl, Br, I, straight chain or branched C₁˜C₆ alkyl, straight chain or branched C₁˜C₆ alkoxy, or —CH₂CH═CH₂;

wherein each R⁵⁰ is independently H; F; Cl; Br; or I;

wherein each R⁵¹ is independently phenyl, wherein said phenyl is optionally substituted with one or two groups, each independently selected from the group consisting of: F, Cl, Br, I, or straight chain or branched C₁˜C₆ alkyl;

wherein each R⁵² is independently phenyl, wherein said phenyl is optionally substituted with one or two groups, each independently selected from the group consisting of: F, Cl, Br, or I;

wherein each R⁵³ is independently straight chain or branched C₁˜C₈ alkyl optionally substituted with C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R⁵⁶ groups; straight chain or branched C₃˜C₅ alkenyl optionally substituted with a phenyl bearing one or more halogen substituents, wherein the olefinic double bond is located β, γ or δ with respect to the ether oxygen; straight chain or branched C₃˜C₅ alkynyl, wherein the alkynyl C≡C bond is located β, γ or δ with respect to the ether oxygen;

wherein each R⁵⁴ is independently H; straight chain or branched C₁˜C₆ alkyl, said alkyl being optionally substituted with one or two moieties selected from the group consisting of F, Cl, Br, and I; C₆˜C₁₄ aryl, said aryl being optionally substituted with one or two moieties independently selected from the group consisting of F, Cl, Br, I, straight chain or branched C₁˜C₆ alkyl, and straight chain or branched C₁˜C₆ alkoxy; C₇˜C₁₉ aralkyl; C₃˜C₁₀ cycloalkyl; or —CH₂R⁶⁴, wherein said R⁶⁴ is straight chain or branched C₂˜C₄ alkenyl or alkynyl;

wherein each R⁵⁵ is independently C₃˜C₁₀ cycloalkyl, said cycloalkyl being optionally substituted with one to three R⁵⁶ groups; C₆˜C₁₄ aryl containing one, two or three rings, said aryl being optionally substituted with one to three R⁵⁶ groups; 3˜10-membered heterocycle containing one or two rings and one or more N, O or S atoms, wherein such heterocycle may be aromatic or non-aromatic, said heterocycle being optionally substituted with one to three R⁵⁶ groups; straight chain or branched C₁˜C₂₀ alkyl; straight chain or branched C₂˜C₁₂ alkenyl; straight chain or branched C₂˜C₄ alkenyl substituted with phenyl or 1- or 2-naphthyl wherein said phenyl or naphthyl is optionally substituted with one to three R⁵⁶ groups; straight chain or branched C₂˜C₁₂ alkynyl; straight chain or branched C₁˜C₄ alkyl substituted with C₃˜C₈ cycloalkyl, C₆˜C₁₄ aryl containing one, two or three rings or 3˜10-membered heterocycle containing one or two rings and one or more N, O or S atoms, wherein such heterocycle may be aromatic or non-aromatic, said cycloalkyl, aryl or heterocycle being optionally substituted with one to three R⁵⁶ groups; phenyl or 1- or 2-naphthyl wherein said phenyl or naphthyl is optionally substituted with one to three R⁵⁷ groups;

wherein each R⁵⁶ is independently F; Cl; Br; I; straight chain or branched C₁˜C₆ alkyl; straight chain or branched C₁˜C₆ alkoxy; straight chain or branched C₁˜C₆ alkylthio, alkylthionyl or alkylsulfonyl; C₃˜C₁₀ cycloalkyl; C₃˜C₁₀ cycloalkyloxy; C₃˜C₁₀ cycloalkylamino; C₃˜C₁₀ cycloalkylthio, cycloalkylthionyl or cycloalkylsulfonyl; C₆˜C₁₄ aryl; C₆˜C₁₄ aryloxy; C₆˜C₁₄ arylamino; C₆˜C₁₄ arylthio, arylthionyl or arylsulfonyl; C₇˜C₁₉ aralkyl; C₇˜C₁₉ aralkyloxy; C₇˜C₁₉ aralkylamino; C₇˜C₁₉ aralkylthio, aralkylthionyl or aralkylsulfonyl; 3˜10-membered heterocycle containing one or two rings and one or more N, O or S atoms, wherein such heterocycle may be aromatic or non-aromatic; 3˜10-membered heterocycle-oxy, heterocycle-amino, heterocycle-thio, heterocycle-thionyl, or heterocycle-sulfonyl, wherein said heterocycle contains one or two rings and one or more N, O or S atoms, wherein said heterocycle may be aromatic or non-aromatic; methylenedioxy; —C≡N; —NO₂; —CF₃; —N(R⁵⁴)₂; —OR⁵⁴; —SH; ═O; —C(O)R⁵⁴; —C(O)OR⁵⁴; —OC(O)R⁵⁴; —C(O)N(R⁵⁴)₂; —N(R⁵⁴)C(O)R⁵⁴; —N(R⁵⁴)C(O)OR⁵⁴; —OC(O)N(R⁵⁴)₂; —N(R⁵⁴)C(O)N(R⁵⁴)₂; —OC(O)OR⁵⁴; —C(O)N(R⁵⁴)N(R⁵⁴)₂; —C(O)N(R⁵⁴)OR⁵⁴; —C(O)N(R⁵⁴)OH; ═S; —C(S)R⁵⁴; —C(S)OR⁵⁴; —OC(S)R⁵⁴; —C(S)N(R⁵⁴)₂; —N(R⁵⁴)C(S)R⁵⁴; —N(R⁵⁴)C(S)OR⁵⁴; —OC(S)N(R⁵⁴)₂; —N(R⁵⁴)C(S)N(R⁵⁴)₂; —C(═NH)R⁵⁴; —C(═NH)N(R⁵⁴)₂; —C(═NH)N(R⁵⁴)N(R⁵⁴)₂; —C(═NH)N(R⁵⁴)OR⁵⁴; C(═NH)N(R⁵⁴)OH; —C(═N—OR⁵⁴)NH₂; —S(O)₂ N(R⁵⁴)₂; —NR⁵⁴S(O)₂R⁵⁴; —C(O)NHS(O)₂R⁵⁴; —S(O)₂R⁵⁴; —SO₃H; or —PO₃H₂;

wherein each R⁵⁷ is independently F; Cl; Br; I; straight chain or branched C₁˜C₆ alkyl; straight chain or branched C₁˜C₆ alkoxy; —C≡N; —CF₃; —NO₂; —NH₂; or —NHC(O)R⁶⁶, wherein said R⁶⁶ is straight chain or branched C₂˜C₁₂ alkyl;

wherein each R⁵⁸ is independently straight chain or branched C₁˜C₂₀ alkyl; C₃˜Cₛ cycloalkyl; straight chain or branched C₁˜C₄ alkyl substituted with phenyl or 1- or 2-naphthyl wherein said phenyl or naphthyl is optionally substituted with one to three R⁵⁶ groups; or phenyl or 1- or 2-naphthyl wherein said phenyl or naphthyl is optionally substituted with one to three R⁵⁷ groups;

wherein each R⁵⁹ is independently straight chain or branched C₁˜C₇ alkyl, alkenyl or alkynyl, said alkyl, alkenyl or alkynyl being substituted with one or more —R⁶⁰-R⁶¹;

wherein each R⁶⁰ is independently a covalent bond or —O—;

wherein each R⁶¹ is independently C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R³⁰ groups;

wherein each R⁶² is independently a covalent bond; —CH₂—CH₂—; —CH═CH—; —O—; or —S—;

wherein each R⁶⁵ is independently straight chain or branched C₁˜C₁₂ acyclic hydrocarbon group, which may be interrupted by one or more —O—, —S—, —S(O)—, —S(O)₂—, said hydrocarbon group optionally containing one or more C═C or C≡C bonds, said hydrocarbon group having no two heteroatoms adjacent and no heteroatom adjacent to a non-aromatic C═C or C≡C bond; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;

wherein each R⁶⁷ is independently H; F; or straight chain or branched C₁˜C₆ alkyl, said alkyl group being optionally substituted by one or more R³⁰;

wherein each R⁶⁸ is independently R³⁶, R⁵⁴ or R⁶¹;

wherein each R⁶⁹ is independently H; or straight chain or branched C₁˜C₆ alkyl, said alkyl group being optionally substituted by one or more R³⁰;

wherein each R⁷⁰ is independently H; straight chain or branched C₁˜C₄ alkyl; straight chain or branched C₁˜C₄ halogenoalkyl exemplified by, but not limited to —CF₃, —CF₂CF₃, —CH₂CF₃, —CHF₂, —CH₂F, —CH₂Cl, —CH₂Br; or cyclopropyl;

wherein each R⁷¹ is independently R⁵⁶; straight chain or branched C₁-C₄ halogenoalkyl exemplified by, but not limited to —CF₃, —CF₂CF₃, —CH₂CF₃, —CHF₂, —CH₂F, —CH₂Cl, —CH₂Br; hydroxymethyl; —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²; and additionally one of the R⁷¹ groups can also represent 1-pyrrolyl; 1-imidazolyl; 1H-1,2,4-triazol-1-yl; 5-tetrazolyl (optionally substituted with straight chain or branched C₁˜C₄ alkyl); 1-pyrrolidinyl; 4-morpholinyl; 4-morpholinyl-N-oxide; —OR⁷⁶; —S(O)ᵣR⁷⁶; —N(R⁷²)R⁷⁶; —C(O)R⁷⁶;

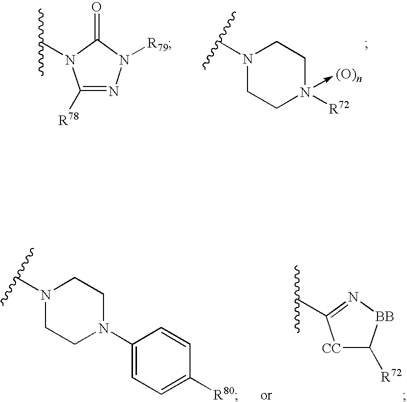

wherein each R⁷² is independently H; straight chain or branched C₁˜C₄ alkyl; C₃˜C₆ cycloalkyl; or straight chain or branched C₁˜C₄ alkyl substituted with phenyl, said phenyl being optionally substituted with one or more halogen, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy;

wherein each R⁷³ is independently H; straight chain or branched C₁˜C₄ alkyl; C₃˜C₆ cycloalkyl; —C(O)R⁷²; or —C(O)CF₃;

wherein each R⁷⁴ is independently straight chain or branched C₁˜C₄ alkyl;

wherein each R⁷⁵ is independently H; —C(O)NH₂; —C(O)CH₃; —C≡N; —SO₂ NHR⁷²; —SO₂R⁷²; —OR⁷²; —OC(O)R⁷²; or straight chain or branched —(C₁˜C₄ alkyl)-NH₂;

wherein each R⁷⁶ is independently phenyl optionally substituted with one or more R⁷⁷ groups;

wherein each R⁷⁷ is independently straight chain or branched C₁˜C₄ alkyl; C₃˜C₆ cycloalkyl; straight chain or branched C₁˜C₄ halogenoalkyl; straight chain or branched C₁˜C₄ alkoxy; straight chain or branched C₁˜C₄ halogenoalkoxy; F; Cl; Br; I; —C≡N; —NO₂; —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²;

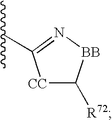

or phenyl, said phenyl being optionally substituted with one or more F, Cl, Br, I, —C≡N, —NO₂, straight chain or branched C₁'C₄ alkyl, straight chain or branched C₁˜C₄ halogenoalkyl, straight chain or branched C₁˜C₄ alkoxy, or straight chain or branched C₁˜C₄ halogenoalkoxy;

wherein each R⁷⁸ is independently H or —CH₃;

wherein each R⁷⁹ is independently H; isopropyl; cyclopentyl; cyclopropyl; 2-butyl; 3-pentyl; 3-hydroxy-2-butyl; or 2-hydroxy-3-pentyl;

wherein each R⁸⁰ is independently F; Cl; Br; I; —C≡N; —NO₂; —NH₂; straight chain or branched C₁˜C₄ halogenoalkyl; straight chain or branched C₁˜C₄ halogenoalkoxy; or

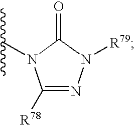

wherein each R⁸¹ is independently alkyloxymethyl wherein said alkyl group is straight chain or branched and has from 1 to 10 carbon atoms; alkenyl or alkenyloxymethyl wherein said alkenyl group is straight chain or branched and has from 2 to 10 carbon atoms; hydroxymethyl; 2-propynyloxymethyl; halomethyl; arylmethyl and aryloxymethyl wherein said aryl is phenyl, substituted phenyl, naphthalenyl or mono- or di-halo-naphthalenyl and wherein said substituted phenyl is phenyl having from 1 to 3 substituents independently selected from the group consisting of halogen, straight chain or branched C₁˜C₆ alkyl, straight chain or branched C₁˜C₆ alkoxy, —NO₂, phenyl, phenylmethyl, benzoyl, halobenzoyl, —C(O)R⁴², —C(O)OR⁴², and —CF₃, with the proviso that when multiple substituents are present only 1 thereof may be selected from the group consisting of phenyl, phenylmethyl, benzoyl and halobenzoyl;

wherein each R⁸² is independently H or —OR⁸⁴;

wherein each R⁸³ is independently H; F; Cl; Br; R⁷⁴; —OR⁷⁴; —SR⁷⁴; —CH₂SC(═O)R⁷⁴; —COOH; or —C(O)OCH₃;

wherein each R⁸⁴ is independently a group that is easily hydrolyzable under physiological conditions, preferably the acyl residue of an amino acid or a group represented by the formula —C(═O)R⁸⁵ or —P(═O)(OR⁸⁶)₂; wherein R⁸⁴ is exemplified by, but not limited to, formyl, acetyl, propionyl, isobutyryl, pivaloyl, succinoyl, benzoyl, nicotinyl, phosphoryl, dimethylphosphoryl, aminoacetyl, 3-aminopropionyl, 4-aminobutyryl, (2-amino-acetylamino)-acetyl, (S)-2,5-diaminopentoyl, (S)-2-aminopropionyl, (S)-pyrrolidine-2-carbonyl, (methylamino)acetyl, (propylamino)acetyl, (S)-2-(methylamino)propionyl, 3-(methylamino)propionyl, (S)-2-amino-3-methylbutanoyl, (isopropylamino)acetyl, (2S)-2-(ethylamino)propionyl, (ethylamino)acetyl and the like;

wherein each R⁸⁵ is independently H; —OR⁷⁴; or straight chain or branched C₁˜C₆ alkyl or alkenyl, wherein said alkyl or alkenyl may be optionally interrupted by a hydrolyzable functional group such as carboxamide or ester, wherein said alkyl or alkenyl may be optionally substituted with —COOH, —NH₂, —NHR⁷⁴, —N(R⁷⁴)₂, or R⁶¹, wherein R⁶¹ is exemplified by, but not limited to, phenyl, methoxyphenyl, pyridyl, pyrazinyl or furyl;

wherein each R⁸⁶ is independently H or R⁷⁴;

wherein each R⁸⁷ is independently straight chain or branched C₁˜C₅ alkyl; R⁶¹; pyridyl; pyrrolidinyl; or -DD-NH—R⁸⁹;

wherein each R⁸⁸ is independently H; F; Cl; Br; or —OR⁷⁴;

wherein each DD is independently straight chain or branched C₁˜C₄ alkylene; —CH₂NHC(═O)CH₂—; —CH(NH₂)CH₂CH₂CH₂—;

wherein each R⁸⁹ is independently H or straight chain or branched C₁˜C₅ alkyl;

wherein each BB is independently —CH₂— or —C(═O)—;

wherein each CC is independently NH or O;

wherein each EE is independently O, S or N—R⁹¹;

wherein each R⁹⁰ is independently tetrahydrofuranyl-(C₁˜C₆ alkyl); or C₁˜C₆ alkyl, C₃˜C₆ cycloalkyl, phenyl-(C₁˜C₆ alkyl) or (C₃—-C₆ cycloalkyl)-(C₁˜C₆ alkyl) all substituted on the C₁˜C₆ alkyl and/or C₃˜C₆ cycloalkyl moiety with ═O, ═S or with one or two radicals of formula KK—R⁹²; wherein said tetrahydrofuranyl is tetrahydrofuran-2-yl or tetrahydrofuran-3-yl; wherein said phenyl is optionally substituted with one to three substituents each independently selected from the group consisting of F, Cl, Br, I, —C≡N, —NO₂, —NH₂, C₁˜C₆ alkyl, C₁˜C₆ alkoxy, and —CF₃; and wherein all said C₁˜C₆ alkyl moieties are straight chain or branched;

wherein each KK is independently O or S;

wherein each R⁹¹ is independently H or straight chain or branched C₁˜C₆ alkyl;

wherein each R⁹² is independently H; straight chain or branched C₁˜C₆ alkyl; C₃˜C₆ cycloalkyl; tetrahydro-2H-pyran-2-yl; phenyl, wherein said phenyl is optionally substituted with one to three substituents each independently selected from the group consisting of F, Cl, Br, I, —C≡N, —NO₂, —NH₂, straight chain or branched C₁˜C₆ alkyl, straight chain or branched C₁˜C₆ alkoxy, and —CF₃; or where R⁹⁰ is substituted with two KK—R⁹² radicals, two R⁹² radicals, taken together, may form a bivalent radical of formula —CH₂—, —CH(CH₃)—, —C(CH³)₂—, —CH₂CH₂—, —CH(CH₃)CH₂—, —CH₂CH₂CH₂—;

wherein each FF is independently —C(═O)—; NR⁹¹; or —CH₂—, optionally substituted with up to two radicals selected from the group consisting of straight chain or branched C₁˜C₆ alkyl and phenyl, said phenyl being optionally substituted with one to three substituents each independently selected from the group consisting of F, Cl, Br, I, —C≡N, —NO₂, —NH₂, C₁˜C₆ alkyl, C₁˜C₆ alkoxy, and —CF₃;

wherein each GG is independently —C(═O)—; or —CH₂—, optionally substituted with up to two radicals selected from the group consisting of straight chain or branched C₁˜C₆ alkyl and straight chain or branched C₁˜C₆ alkoxy;

or FF and GG, taken together, form a bivalent radical of formula —N═CH— (FFGG′);

wherein HH has the same meaning as FF;

wherein JJ has the same meaning as GG;

or HH and JJ, taken together, form a bivalent radical of formula —N═CH— (FFGG′) or —CH═CH— (HHJJ′);

wherein the nitrogen atom in the bivalent radical FFGG′ is connected to NR⁹⁰; wherein one hydrogen in said radical FFGG′ and up to two hydrogens in radical HHJJ′ may be replaced by a straight chain or branched C₁˜C₆ alkyl radical;

wherein each R⁹³ is independently H or C₁˜C₄ alkyl which is unsubstituted or substituted by 1-3 substituents each independently selected from the group consisting of halogen, hydroxyl, C₁˜C₄ alkoxy, and amino; or two R⁹³ groups attached to the same carbon atom together form a lower alkylene group exemplified by, but not limited to —CH₂CH₂—, —CH₂CH₂CH₂—, —CH₂CH₂CH₂CH₂— and the like;

wherein each R⁹⁴ is independently H or C₁˜C_{I} alkyl which is unsubstituted or substituted by 1-3 substituents each independently selected from the group consisting of halogen, hydroxyl, C₁˜C_{I} alkoxy, and amino; or two R⁹⁴ groups attached to the same carbon atom together form ═S;

wherein each LL is independently a covalent bond; —C(═O)—; —C(═S)—; —SO₂—; or —N═N—;

wherein each R⁹⁵ is independently selected from the group consisting of

i) hydrogen,

ii) CN

iii) CHO

iv) phenyl which is unsubstituted or substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) C₁˜C₄ alkoxy, (3) halogen, (4) formyl, (5) carboxyl, (6) C₁˜C₄ acyloxy, (7) C₁˜C₄ alkoxycarbonylamino, (8) phenyl- or naphthyl-oxycarbonylamino, (9) semicarbazido, (10) formamido, (11) thioformamido, (12) hydroxy, (13) nitro, (14) amino, (15) furyl, (16) triazolyl, (17) thienyl, (18) oxazolyl, (19) imidazolyl and (20) triazolone-yl,

v) a 5- or 6-membered monocyclic or 8- to 10-membered bicyclic heterocycle having 1-4 heteroatoms each independently selected from the group consisting of N, O and S, which heterocycle is unsubstituted or ring-substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) benzyl which is unsubstituted or substituted with 1-3 substituents selected from the group consisting of C₁˜C₄ alkyl, CF₃, halogen and OCF₃, (3) halogen, (4) hydroxy, (5) nitro, (6) amino, (7) C₁˜C₄ acylamino, (8) formyl, (9) formamido, (10) thioformamido, (11) C₁˜C₄ alkoxycarbonylamino, (12) phenyl- or naphthyl-oxycarbonylamino and (13) semicarbazido,

vi) NHR⁹⁶ wherein R⁹⁶ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) CᵣC₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl, and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of hydroxy, halogen, amino and carboxyl,

vii) OR⁹⁷ wherein R⁹⁷ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8 to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, and

viii) a group of the formula

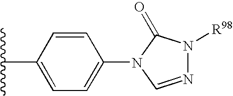

wherein R⁹⁸ is selected from the group consisting of (1) hydrogen, (2) C₁˜C₁₀ alkyl which is unsubstituted or substituted by 1-5 substituents each independently selected from the group consisting of halogen, hydroxy, C₁˜C₄ alkoxy and amino, (3) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl. (e) carboxyl, (f) C₁˜C₄ acyloxy, (g) C₁˜C₄ alkoxycarbonylamino, (h) phenyl- or naphthyl-oxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl, (t) triazolone-yl, (u) CF₃ and (v) OCF₃, (4) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8- to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, (5) phenyl(C₁˜C₄ alkyl) which is unsubstituted or ring-substituted with 1-3 substituents each independently selected from the group consisting of (a) C₁˜C₅ alkyl which is unsubstituted or substituted with 1-3 substituents each independently selected from the group consisting of halogen, hydroxy, C₁˜C₄ alkoxy and amino, (b) halogen, (c) halo(C₁˜C₄ alkyl), (d) C₁˜C₄ alkoxy, (e) hydroxy, (f) amino, (g) carboxyl, (h) trifluormethoxyl, (i) trifluoromethyl, (j) tetrafluoroethyl, (k) tetrafluoroethoxyl, (l) tetrafluoropropyl and (m) tetrafluoropropoxyl, (6) naphthyl(C₁˜C₄ alkyl) which may be substituted with 1-6 substituents selected from (a) C₁˜C₄ alkyl which is unsubstituted or substituted with 1-3 substituents each independently selected from the group consisting of halogen, hydroxy, C₁˜C₄ alkoxy and amino, (b) halogen, (c) (C₁˜C₄ alkyl)halo, (d) C₁˜C₄ alkoxy, (e) hydroxy, (f) amino, (g) carboxyl, (h) trifluoromethoxyl, (i) trifluoromethyl, (j) tetrafluoroethyl, (k) tetrafluoroethoxyl, (l) tetrafluoropropyl and (m) tetrafluoropropoxyl, (7) methoxyl, (8) trifluoromethoxyl, (9) trifluoromethyl, (10) trifluoroethyl, (11) tetrafluoroethyl, (12) tetrafluoroethoxyl, (13) tetrafluoropropyl and (14) tetrafluoropropoxyl;

wherein each MM is independently an ethylene group optionally substituted with R91; or MM is —NN═OO—, wherein NN and OO are the same or different and are each independently N or CR⁹¹;

wherein each R⁹⁹ is independently a five-membered aromatic heterocyclic group containing one to four nitrogen atoms as ring-constituent atoms, which may further contain a heteroatom selected from sulfur or oxygen as a ring-constituent atom, said heterocyclic group being optionally substituted on the ring-constituent carbon and/or nitrogen atoms with one to three substituents selected from the group consisting of R³⁰ and R⁵⁶;

wherein each R¹⁰⁰ is independently —R⁵⁴ or —OR⁵⁴;

wherein each R¹⁰¹ is independently H; F; Cl; Br; or I;

wherein each R¹⁰² is independently F; Cl; Br; I; —SR¹⁰³; or —OR¹⁰⁴;

wherein each R¹⁰³ is independently straight chain or branched C₁˜C₁₂ alkyl, phenyl, furfuryl, —CH₂R⁶⁴, or benzyl, wherein said benzyl is optionally substituted by one to three R⁵⁶ substituents which may be the same or different;

wherein each R¹⁰⁴ is independently straight chain or branched C₁˜C₁₂ alkyl; or phenyl;

wherein each R¹⁰⁵ is independently H; R²⁹; straight chain or branched C₁˜C₈ alkyl; ═CH₂; straight chain or branched C₁˜C₆ alkenyl; straight chain or branched C₁˜C₆ alkyl substituted with one or two groups selected from the group consisting of F, Cl, Br, I, —C≡N, straight chain or branched C₁˜C₆ alkoxy, straight chain or branched C₁˜C₆ alkylthio, —C(═O)NH₂, —C(═O)—CH₂R⁶⁴, —C(═O)R⁴²; benzyl; methylenedioxybenzyl; C₇˜C₁₀ phenoxyalkyl optionally substituted with one to three halogen atoms; naphthyl; pyridyl optionally substituted with one to three substituents independently selected from the group consisting of halogen and R³⁰;

wherein each R¹⁰⁶ is independently H, a pharmaceutically acceptable cation, or null (affording an azolium phosphate zwitterion);

wherein each chiral center independently may possess any relative or absolute stereoconfiguration or be any mixture thereof, unless specified otherwise herein; and

wherein each olefinic C═C bond that is capable of E/Z isomerism independently may possess either the E or Z stereoconfiguration or be any mixture thereof, unless specified otherwise herein.

In some embodiments, the at least one compound of Formula IV is diastereomerically pure.

In some embodiments, the at least one compound of Formula IV is a mixture of diastereomers in any ratio.

In some embodiments, the at least one compound of Formula IV is enantiomerically pure.

In some embodiments, the at least one compound of Formula IV is a mixture of enantiomers in any ratio, including a racemate.

In some embodiments, the at least one compound of Formula IV is diastereomerically and enantiomerically pure.

In some embodiments, the at least one compound of Formula IV is a mixture of diastereomers and enantiomers in any ratio.

In some embodiments, the at least one compound of Formula IV has one or more hydrogen atoms replaced by deuterium.

In some embodiments, the at least one compound of Formula IV is a pharmaceutically acceptable salt derived from the combination of a compound of Formula IV with an acid or a base.

In some embodiments, the at least one compound of Formula IV is an N-oxide form of an amine.

In some embodiments, the at least one compound of Formula IV is a zwitterion.

In some embodiments, the at least one compound of Formula IV is a tautomer of a structure described herein.

Examples of compounds of Formula IV include, but are not limited to, the antifungal agents bifonazole, butoconazole, croconazole, econazole, enilconazole, clotrimazole, flutrimazole, isoconazole, fenticonazole, ketoconazole, sulconazole, tioconazole, oxiconazole, sertaconazole, miconazole, chlormidazole, omoconazole, posaconazole, elubiol, voriconazole, eberconazole, CAS RN 214543-30-3, democonazole, genaconazole, vibunazole, lombazole, pramiconazole, T 8581, fluconazole, albaconazole, tiabendazole, parconazole, ravuconazole, saperconazole, itraconazole, hydroxyitraconazole, TAK 456, terconazole, SYN 2869, neticonazole, NND-502, lanoconazole, fosfluconazole; and the thromboxane synthase inhibitors 7-(1-imidazolypheptanoic acid, ozagrel, and 1-benzyl imidazole.

Other compounds of interest not contained within Formula IV include the antifungal chlordantoin; non-azole thromboxane synthase inhibitors, such as terbinafine, furegrelate, BM 567, and analogs of terbinafine, such as amorolfine, butenafine, naftifine; and Epoxygenase inhibitors, such as MS-PPOH, and PPOH.

In some embodiments, an imiilazole analog of the described invention is a compound of Formula V, or a hydrate, solvate, salt, zwitterion, N-oxide, tautomer, prodrug or metabolite of Formula V having the structure:

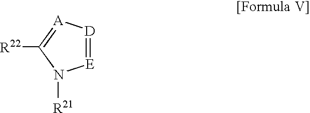

wherein each A is independently N or —NR²⁰;

wherein each D is independently CR²³;

wherein each E is independently N or CR²⁴;

wherein R²³ is H;

wherein R²⁴ is H;

or wherein R²³ and R²⁴ together are —CH═CH—CH═CH—;

wherein R²⁰ is H and the compound of Formula V is an imidazolium or triazolium salt with a pharmaceutically acceptable counter anion; or

wherein R²⁰ is a moiety that is readily cleaved in vivo, exemplified by, but not limited to, —CH₂OC(O)CH₃, and the compound of Formula V is a prodrug and an imidazolium or triazolium salt with a pharmaceutically acceptable counter anion;

wherein R²² is H; —CH₃; or

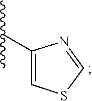

wherein R²¹ is H; —(CR²⁵R²⁶)ₘ—CR²⁷R²⁸-Q-R²⁹;

wherein each m is independently 0 or 1;

wherein R²⁵ is H; —CH₃; or —C≡N;

wherein R²⁶ is H;

wherein R²⁷ is -T-U—V;

wherein R²⁸ is H; OH; or

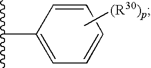

wherein each Q is independently a covalent bond or —S—;

wherein R²⁹ is

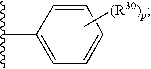

or straight chain or branched C₃˜C₁₋₁₃ alkyl optionally substituted with —CO₂H;

wherein each p is independently an integer from 0 to 3 inclusive;

wherein each T is independently a covalent bond; —CH₂CH₂—; —OCH₂—; —CH₂O—; —SCH₂—; —CH₂S—; —OCH₂CH₂O—; —CH(CH₃); —CH₂—; or —CF₂—;

wherein each U is independently a covalent bond;

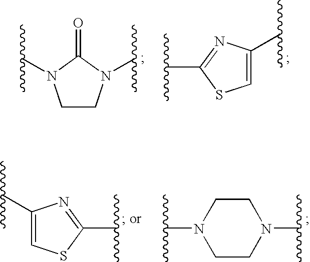

wherein each V is independently H; —S(O)₂CH₃; —C(O)NH₂; —CH₂C≡CH; —CH═CH₂;

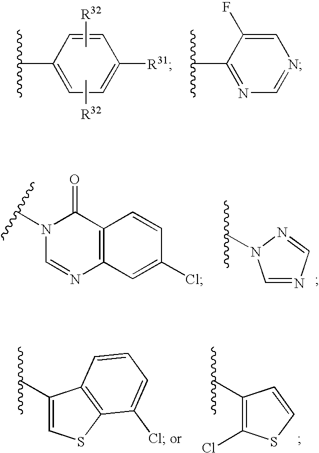

wherein each R³⁰ is independently F, Cl, —CH═CH—CO₂H;

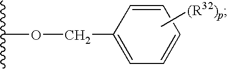

or straight chain or branched C₁˜C₁₀ alkoxy;

wherein each R³¹ is independently H; F; Cl; Br; I; —C≡N;

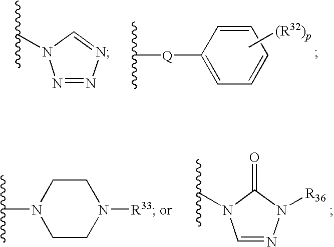

wherein each R³² is independently H; F; Cl; or —OCF₃;

wherein each R³³ is independently straight chain or branched C₁˜C₆ alkyl; —C(O)R³⁴; —C(O)OR³⁴;

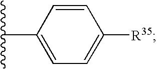

wherein each R³⁴ is independently straight chain or branched C₁˜C₆ alkyl;

wherein each R³⁵ is independently H;

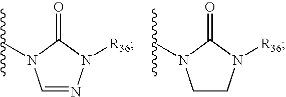

wherein each R³⁶ is independently straight chain or branched C₁˜C₆ alkyl optionally substituted with OH; or

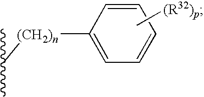

wherein each n is independently 0 or 1;

wherein R²⁶ and R²⁸ together with the two carbon atoms to which they attach may be C═C;

wherein R²⁷ and R²⁸ together may be

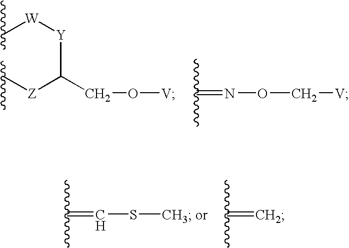

wherein W is —CH₂— and Y is —CH₂—; W is —O— and Y is —CH₂; or W is —CH₂— and Y is —O—;

wherein Z is —CH₂— or —O—;

wherein R²⁷ together with Q R²⁹ may be

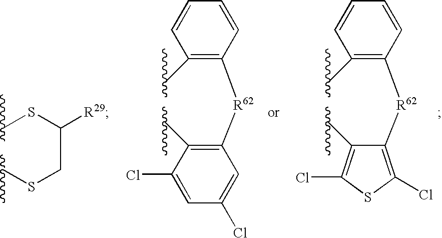

wherein each R⁶² is independently —CH₂CH₂— or —CH═CH—.

In some embodiments, each chiral center independently possesses any relative or absolute stereoconfiguration or is any mixture thereof, unless specified otherwise herein.

In some embodiments, each olefinic C═C bond that is capable of E/Z isomerism independently can possess either the E or Z stereoconfiguration or be any mixture thereof, unless specified otherwise herein.

In some embodiments, the at least one compound of Formula V is diastereomerically pure.

In some embodiments, the at least one compound of Formula V is a mixture of diastereomers in any ratio.

In some embodiments, the at least one compound of Formula V is enantiomerically pure.

In some embodiments, the at least one compound of Formula V is a mixture of enantiomers in any ratio, including a racemate.

In some embodiments, the at least one compound of Formula V is diastereomerically and enantiomerically pure.

In some embodiments, the at least one compound of Formula V is a mixture of diastereomers and enantiomers in any ratio.

In some embodiments, the at least one compound of Formula V has one or more hydrogen atoms replaced by deuterium.

In some embodiments, the at least one compound of Formula V is a pharmaceutically acceptable salt derived from the combination of a compound of formula IV with an acid or a base.

In some embodiments, the at least one compound of Formula V is an N-oxide form of an amine described herein.

In some embodiments, the at least one compound of Formula V is a zwitterion.

In some embodiments, the at least one compound of Formula V is a tautomer of a structure described herein.

Examples of imidazole analogs according to Formula V include, but are not limited to, histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole.

According to one embodiment, the compound of Formula V, is miconazole or a solvate, a hydrate, a salt, a zwitterion, an N-oxide, a tautomer, a prodrug or a metabolite of miconazole.

The compounds of the described invention may be formulated into pharmaceutical compositions. As used herein, “pharmaceutically acceptable salts” include, but are not limited to, those formed with free amino groups such as those derived from hydrochloric, phosphoric, sulfuric, acetic, oxalic, tartaric acids, etc., and those formed with free carboxyl groups, including, but not limited to, those derived from sodium, potassium, ammonium, calcium, ferric hydroxides, isopropylamine, triethylamine, 2-ethylamino ethanol, histidine, procaine, and the like.

Compositions of the described invention may be prepared readily using technology which is known in the art such as described in Remington's Pharmaceutical Sciences, 18th or 19th editions, published by the Mack Publishing Company of Easton, Pa., which is incorporated herein by reference.

The compounds of the described invention may be used in methods to treat an epithelial-related condition selected from the group consisting of sparse hair growth, thin hair growth, short hair growth, alopecia and hair depigmentation, the method comprising the steps (a) formulating a composition comprising at least one prostaglandin analog compound of the described invention; and (b) administering the composition to a subject in need thereof.

The described invention provides topical compositions for treating an epithelial-related condition selected from the group consisting of sparse hair growth, thin hair growth, short hair growth, alopecia and hair depigmentation in a subject in need of treatment thereof, including a human, that includes a compound of the described invention and a carrier.

The described invention further provides compositions useful for treating a condition of the skin or a mucosal membrane relating to sparse hair growth, thin hair growth, short hair growth, alopecia, and hair depigmentation, such as, but not limited to, that of a nose, an eye, a face, and a scalp.

In another embodiment, the described invention provides a topical pharmaceutical composition for treating an epithelial-related condition selected from the group consisting of sparse hair growth, thin hair growth, short hair growth, alopecia and hair depigmentation in a subject, including a human, in need of treatment thereof that includes at least one prostaglandin analog compound of the described invention and a carrier.

In a further embodiment, the described invention provides a topical cosmetic composition for treating an epithelial-related condition selected from the group consisting of sparse hair growth, thin hair growth, short hair growth, alopecia and hair depigmentation in a subject, including a human, in need of treatment thereof that includes at least one prostaglandin analog compound of the described invention and a carrier.

In another embodiment, provided herein is a method of treating an epithelial-related condition selected from the group consisting of sparse hair growth, thin hair growth, short hair growth, alopecia and hair depigmentation, the method including the step of topically applying onto an epithelial surface of a mammal, including a human, in need thereof a pharmaceutically effective amount of a composition that includes at least one prostaglandin compound of the described invention and a carrier.

In a further embodiment, the described invention provides a method of treating an epithelial-related condition selected from the group consisting of sparse hair growth, thin hair growth, short hair growth, alopecia and hair depigmentation, the method including the step of topically applying onto a surface of a subject, including a human, in need thereof a cosmetically effective amount of a composition that includes at least one prostaglandin analog compound of the described invention and a carrier.

In a further embodiment, the invention provides a method of preparing a topical composition, the method including the step of admixing at least one prostaglandin analog compound of the described invention and a carrier.

The described compositions containing at least one prostaglandin analog compound of the described invention can be used effectively in topical applications to treat cosmetic conditions.

The compositions of the described invention may be usefully employed in cosmetic, cosmeceutical and general skincare compositions as well as in pharmaceutical compositions.

In one embodiment, the composition of the described invention is a pharmaceutical composition.

In another embodiment, the composition of the described invention is a cosmetic composition.

In another embodiment, the composition of the described invention is a cosmeceutical composition.

In another embodiment, the composition of the described invention is a cosmetic composition that restores hair pigmentation to depigmented hair.

Hair color is the result of pigmentation of the hair. Stem cells at the base of hair follicles are responsible for producing melanocytes, the cells that produce and store pigment in hair and skin. At some point in the aging process, these cells make less and less pigment, until the hair has very little pigment. White hair has no pigment, while gray hair has some pigment.

The graying process usually is gradual. However, it has been noted that tobacco smoking may cause premature graying. Additionally, there are a number of medical conditions that affect hair color. For example, albinism is a genetic abnormality in which no pigment is found in human hair, eyes or skin; vitiligo is the patchy loss of hair and skin color that may occur as the result of an autoimmune disease; malnutrition, which is reversible with proper nutrition, is known to cause hair to become lighter, thinner, and more brittle; patients suffering from Werner syndrome, a rare autosomal recessive disorder that resembles accelerated aging, develop many of the changes associated with aging as young adults, including the graying and loss of hair; and pernicious anemia due to B12 deficiency also can cause premature graying.

In another aspect of the described invention, the composition of the described invention includes a carrier.

Some non-limiting representative examples of carriers include moisturizing agents or humectants, pH adjusting agents, hair conditioning agents, chelating agents, preservatives, emulsifiers, thickeners, solubilizing agents, penetration enhancers, anti-irritants, colorants and surfactants.

Representative examples of moisturizing or humectant agents that are usable in the described invention include, without limitation, guanidine, glycolic acid and glycolate salts (e.g. ammonium salt and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

As is widely recognized in the art, since the pH of the skin is 5.5, compositions for topical skin application (to avoid irritation) should preferably have a pH value of between pH 4.0 and pH 7.0. In some embodiments, the pH is between pH 5.0 and pH 7.0. In some embodiments, the pH is about pH 5.5. Hence, a pH adjusting composition is typically added to bring the pH of the composition to the desired value. The compositions of the described invention therefore preferably are formulated to have a pH value of about 7.0. Suitable pH adjusting agents include, for example, but are not limited to, one or more adipic acids, glycines, citric acids, calcium hydroxides, magnesium aluminometasilicates, buffers or any combinations thereof.

Suitable hair conditioning agents that can be used in the context of the described invention include, for example, one or more collagens, cationic surfactants, modified silicones, proteins, keratins, dimethicone polyols, quaternary ammonium compounds, halogenated quaternary ammonium compounds, alkoxylated carboxylic acids, alkoxylated alcohols, alkoxylated amides, sorbitan derivatives, esters, polymeric ethers, glyceryl esters, or any combinations thereof.

Hair stimulating agents may be added to the compositions of the described described invention. For example Procapil® (FR 2 791 684 and WO0058347) promotes the visible appearance of thicker and fuller hair and prevents premature hair thinning and hair loss by boosting the synthesis of components at the epidermal junction where the hair anchors to the skin, which helps to anchor the hair follicles more firmly to the scalp. U.S. Pat. No. 6,861,077 describes methods to protect keratinous fibers from extrinsic damages comprising application of compositions comprising at least one plant extract. For example, a plant extract composed of purified glycoproteins obtained from white potatoes (Solanum tuberosum L. is commercially available from SEDERMA, Inc. (France) as Dermolectine® and Capilectine®. ANCRIN® (Sederma), a hydroglycolic solution containing octylbutyrate and glutamine peptide, reduces hair loss by supplying a vegetable substrate to transglutaminases, a group of enzymes known to increase protein reticulation in the scalp and help anchor the hair to the scalp. Capisome™ (Sederma) is a liposome that comprises homotaurine (3-aminopropane sulfonic acid), a bacterial filtrate of biotechnological origin from enterobacteria that contains high levels of peptides and the sulfur-containing amino acids methionine and cysteine; and marine sulfopolysaccharides. (See U.S. Pat. No. 6,376,557, incorporated herein by reference). Capigen™ (Sederma), is a complex that comprises homotaurine (3-aminopropane sulfonic acid), a bacterial filtrate obtained from a strain of microorganisms cultured in a medium comprising selected peptides, with the filtrate containing high levels of peptides, and a sulfomuycopolysaccharide of marine origin, which is a complex of sulfated polysaccharides that are soluble in water and are found in the connective tissue and synovial fluids. Follicusan® (Chemlishes Laboratorium Dr. Kurt Richter GmbH), is composed of a fraction derived from milk, ethyl pantenol, inositol and sulfur-containing amino acids (N-acetylcysteine and N-acetyl methionine in an aqueous alcoholic medium. Anageline® (Silab) contains an extract from white sweet lupine. Cprillisil (Exsymol S.A.M. of Monaco) is a 20% solution of dimethylsilanediol salicilate in butylenes glycol with triethanolamine. Mahanimba is an extract of the flowers and inflorescence of the neem tree (Melia azadirachta) and contains carotinoids, amino acids, phytosterols, mucins, polyacetylenes, and ses quiterpenes. Malkagni is an extract of the seeds, leaves and flowers of the intellect tree (Celastrus paniculata) and contains tannins, mineral salts, saponins, and iridic glycosides. Fitopur B is a complex available from Sederrna, Inc., and comprises extracts of three plants: buchu (Buc hu barosma), henna (Lawsonia inermis), and venus hair (Adiatium capillus-veneris). The essential oil of buchu contains the terpenic oil diosphenol and sulfur compounds. The leaves of henna contain flavonic pigments, includig luteoline and laxanthones, principally lawsone. Venus hair is a small firm native to the south of France; it has diuretic and emollient activity. Peptide-copper complexes containing dipeptides or tripeptides chelated to copper stimulate hair growth. See U.S. Pat. No. 5,538,945 and U.S. Pat. No. 6,017,888, incorporated herein by reference). Hormone replacement therapy (HRT), including administration of micronized progesterone pills and creams and estrogen pills and creams, is used to treat androgenetic alopecia for women. Other such agents are known by persons of skill in the art.

Chelating agents optionally are added to the compositions of the described invention so as to enhance the preservative or preservative system. In some embodiments, the chelating agents are mild agents, such as, for example, ethylenediaminetetraacetic acid (EDTA), EDTA derivatives, or any combination thereof.

Suitable preservatives for use in the compositions of the described composition include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or botanical agents, such as, but not limited to, leuconostocil or radish root ferment filtrate, or any combinations thereof.

In another embodiment, a composition comprising at least one prostaglandin compound of the described invention, a carrier and other, optional ingredients can be dispersed in an emulsion.

In some embodiments, the compositions of the invention may be in the form of an oil-in-water emulsion. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate.

Suitable emulsifiers may be natural materials, finely divided solids, or synthetic materials. Natural emulsifying agents may be derived from either animal or vegetable sources. Those from animal sources include gelatin, egg yolk, casein, wool fat, or cholesterol. Those from vegetable sources include acacia, tragacanth, chondrus, or pectin. Vegetable sources specifically from cellulose derivatives include methyl cellulose and carboxymethyl cellulose to increase the viscosity. Finely divided emulsifiers include bentonite, magnesium hydroxide, aluminum hydroxide, or magnesium trisylicate. Synthetic agents include anionic, cationic or nonionic agents. Particularly useful are sodium lauryl sulfate, benzalkonium chloride or polyethylene glycol 400 monostearate, or any combinations thereof.

Suitable thickeners that can be used in the context of the described invention include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, fatty acids, anionic polymers, and their alkali salts and mixtures thereof.

Representative examples of solubilizing agents that are usable in the context of the described invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEEN® and spans, e.g., TWEEN 80®, which is polysorbate 80, and SPANS (for example, SPAN® 20) which is sorbitan monolaurate, available from Croda International PLC. Other solubilizers that are usable for the compositions of the described invention are, for example, polyoxyethylene sorbitan fatty acid ester, polyoxyethylene n-alkyl ethers, n-alkyl amine n-oxides, polyoxamers, organic solvents, such as acetone, phospholipids and cyclodextrins.

Suitable penetration enhancers usable in the described invention include, but are not limited to, a vegetable oil. Such oils include, for example, safflower oil, cottonseed oil and corn oil.

Additional thickeners, penetration enhancers and other adjuvants may generally be found in Remington's Pharmaceutical Sciences, 18th or 19th editions, published by the Mack Publishing Company of Easton, Pa., which is incorporated herein by reference.

Suitable anti-irritants that can be used in the context of the described invention include, for example, steroidal and non steroidal anti-inflammatory agents or other materials such as aloe vera, chamomile, alpha-bisabolol, cola nitida extract, green tea extract, tea tree oil, licorice extract, allantoin, caffeine or other xanthines, glycyrrhizic acid and its derivatives. Presently known anti-irritants can be divided into water-soluble anti-irritants and water-insoluble anti-irritants. Representative examples of such compositions are described, for example, in U.S. Pat. No. 5,482,710, which is incorporated herein by reference.

Colorants also may be used in the compositions of the invention. Preferred pigments include, but are not limited to, iron oxides, and titanium oxides. Suitable dyes include FD&C approved colorants, D&C approved colorants, and those approved for use in Europe and Japan. See Marmion, D. M., Handbook of US Colorants for Food, Drugs, Cosmetics, and Medical Devices, 3rd ed, 1991 herein incorporated by reference.

Suitable surfactants for use with the inventive compositions include, but are not limited to, sarcosinates, glutamates, sodium alkyl sulfates, ammonium alkyl sulfates, sodium alkyleth sulfates, ammonium alkyleth sulfates, ammonium laureth-n-sulfates, sodium laureth-n-sulfates, isothionates, glycerylether sulfonates, sulfosuccinates and combinations thereof. More preferably, the anionic surfactant is selected from the group consisting of sodium lauroyl sarcosinate, monosodium lauroyl glutamate, sodium alkyl sulfates, ammonium alkyl sulfates, sodium alkyleth sulfates, ammonium alkyleth sulfates, and combinations thereof.

In some embodiments, a pharmaceutically acceptable carrier is included in the composition.

In another embodiment, the compositions of the described invention include a cosmetically acceptable carrier. It will be understood that cosmetically acceptable carriers and pharmaceutically acceptable carriers are similar, if not often identical, in nature.

Suitable pharmaceutically acceptable carriers include water, petroleum jelly (Vaseline™), petroleum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, alcohols, polyols, and the like. Also included are the carriers described hereinabove.

In another embodiment, the pharmaceutically acceptable carrier of the composition of the described invention includes a sustained release or delayed release carrier. The carrier can be any material capable of sustained or delayed release of the compound to provide a more efficient administration resulting in less frequent and/or decreased dosage of the compound, ease of handling, and extended or delayed effects on epithelial-related conditions. Non-limiting examples of such carriers include liposomes, microsponges, microspheres, or microcapsules of natural and synthetic polymers and the like. Liposomes which may enhance the localized delivery of the compounds of the inventive composition within skin layers, may be formed from a variety of phospholipids, such as cholesterol, stearylamines or phosphatidylcholines.

Suitable cosmetically acceptable carriers are described in the CTFA International Cosmetic Ingredient Dictionary and Handbook, 8th edition, edited by Wenninger and Canterbery, (The Cosmetic, Toiletry, and Fragrance Association, Inc., Washington, D.C., 2000), which is herein incorporated by reference. Also included are the carriers described hereinabove.

Mascara

Generally, the term “mascara” refers to a cosmetic used to darken, lighten, color, thicken, lengthen and define eyelashes. Mascara comes in three forms: liquid, cake and cream, is available in many formulas, tints and colors, and may be formulated as a water resistant mascara or a non-water resistant mascara.

Generally, the composition of a water resistant mascara is based on a volatile solvent (for example, isododecane—an isomer of dodecane), animal-derived waxes (for example, beeswax), vegetal based waxes (for example, carnauba wax, rice bran wax, candelila wax), mineral origin wax (for example, ozokerite, paraffin), pigments (for example, iron oxide, ultramarine) and filmifying polymers. Because these mascaras do not contain water-sensitive moieties, they offer an excellent resistance to water and only can be removed with a specific make-up remover that is able to dilute the dried mascara film.

Non-water-resistant mascaras are based on water, soft surfactants (for example, triethanolamine stearate), animal-derived waxes (for example, beeswax), vegetal based waxes (for example, carnauba wax, rice bran wax, candelilla wax), mineral origin waxes (for example, ozokerite, paraffin), pigments (for example, iron oxide, ultramarine), thickening polymers (for example, gum arabic, hydrophobically modified cellulose) and preservatives. They can run under the effect of tears, but are easily removed with some soap and water. Polymers in a water dispersed form (for example, latexes) can bring some level of water resistance to normally non-water resistant mascaras. Waterproof mascaras are similar to oil-based or solvent-based paints, while non water-resistant mascaras behave like water based paints. For intermediate water sensitivity, mascaras and latex-based paints (acrylates) contain polymer dispersions.

The choice of mascara depends on the type of eyelashes (short or long, stiff or curved, poor or bushy, and fair or brown) and of the required effect (lengthened, curved and/or thicker eyelashes). Liquid mascaras are the most popular modern formulation, and they can be divided into water-based, solvent-based and water/solvent hybrid varieties.

Water-based mascaras are formulated from waxes (for example, beeswax, carnauba wax, and synthetic wax), water, pigments, which are often iron oxides, and resins dissolved in water. The water evaporates readily, creating a fast-drying product, which thickens and darkens the eyelashes. Some water-based mascaras, very rich in wax (about 30%), are labeled waterproof or water resistant. To color eyelashes, inorganic pigments are the most commonly used because the vast majority of mascaras are black. The formulae also contain antioxidizers to avoid the rancid smell of fatty substances and preservatives, which protect the eye from any risk of infection. Vitamins and hydrocarbon volatile or silicon solvents also can be used to improve the performance of the makeup.

Concerning the solvent-based mascaras, they are formulated with petroleum distillates to which pigments (for example, iron dioxides, ultramarine blue) and waxes (for example, candellila wax, ozokerite and hydrogenated castor oil) are added, making them waterproof.

If it is clear that the makeup effect depends on the formula, it also is important to consider the type of brush and the diameter of the aperture of the mascara tube. Indeed, it must automatically adjust the quantity of the product on the brush to avoid loads on the eyelashes during application. The packaging also must be totally airtight to avoid the degradation and oxidation of the formulation. Thus, to obtain a good application on the eyelashes, it is necessary to develop a compromise between the mascara formula viscosity and the brush type. A rheological approach can be made through the rheological characterization in situ of mascara pastes with the brushes. This procedure used to quantify the take up of mascara brush in the container allows to visualize the influences of the shaft, the bristle length, and the hardness and pattern on the take up, therefore to characterize the product transfer.

According to some embodiments, the compositions of the described invention may be formulated as a mascara. According to some such embodiments, the mascara is a water-resistant mascara. According to some such embodiments, the mascara is a non-water-resistant mascara.

The mascara compositions of the described invention comprise at least one prostaglandin analog and optionally an imidazole. In some embodiments, the imidazole is miconazole. Within the mascara compositions, agents may be utilized to convert a water dispersible colorant to a non-water dispersible colorant. Accordingly, the mascara compositions also may include at least one cosmetically acceptable hydrophobic colorant. For example, a hydrophobic agent may be coated onto at least one cosmetically acceptable colorant. Hydrophobic agents useful in providing this function include lecithin, dimethicone, dimethicone and mineral oil, polyethylene, isopropyl triisostearyl titanate, calcium stearate, isostearic acid and the combination of squalane, beeswax and lauric acid. All of these specific hydrophobic agents are reported in the CTFA Cosmetic Ingredient Dictionary, Third Edition or Third Edition, Supplement, published by the Cosmetic, Toiletry and Fragrance Association, Inc., Washington, D.C. These dictionary volumes, published 1982 and 1985, respectively, are incorporated herein by reference.

The hydrophobically treated colorants suitable for such compositions include such cosmetically acceptable colorants as carmine, bismuth oxychloride, iron oxides (for example, iron oxide black, iron oxide yellow and iron oxide red), zinc oxide, kaolin, ultramarine blue, ultramarine green, ultramarine pink, ultramarine red, ultramarine violet, chromium hydroxide green, chromium oxide greens, silica and manganese violet.

In some embodiments, a cosmetically acceptable pigment treated with a hydrophobic agent, (i.e., a hydrophobic pigment), comprises between about 1% and about 12% by weight, based on the total weight of the mascara composition. In some embodiments, the hydrophobic pigment comprises between about 2% and about 10% by weight, based on the total weight of the mascara composition. In some embodiments, the hydrophobic pigment is present in a concentration in the range of between about 2% and about 8% by weight, based on the total weight of the mascara composition. In some embodiments, the hydrophobic pigment constitutes between about 2% and about 5% by weight, based on the total weight of the mascara composition.

The mascara composition may further comprise a film forming resin. Film forming resins include polyvinyl alcohol, polyvinyl acetate, PVP, ammonium acrylates copolymer, cellulose gum, carboxymethyl hydroxyethylcellulose, acrylate/ammonium methacrylate copolymer and acrylic/acrylate copolymer. These resins are fully defined in the CTFA Cosmetic Ingredient Dictionary, Third Edition and Third Edition, Supplement which are again incorporated herein by reference.

The film forming resin constituent of the cosmetic composition represents about 0.5% to about 4% by weight. In some embodiments, the film forming resin is present in the composition in a concentration in the range of between about 0.8% and about 2.5% by weight. In some embodiments, the film forming resin is present in the composition in a concentration in the range of between about 0.9% and about 1.5% by weight. Those skilled in the art appreciate that the film forming polymers used in the mascara composition of the described invention do not produce sticky type products.

The mascara composition may further comprise a water dispersible thickening agent. In some embodiments, the water dispersible thickening agent is the combination of a base and a polymeric acid. Bases that may be employed to form the water dispersible thickening agent, include, but are not limited to, triethanolamine, isopropanolamine, diisopropanolamine, ethanolamine, sodium hydroxide and potassium hydroxide. Polymeric acids that may be used to form the water dispersible thickening agent are polymers of acrylic acid cross linked with polyfunctional agents such as Carbomer® 910, Carbomer® 934, Carbomer® 934P, Carbomer® 940 and Carbomer® 941.

In formulating such a mascara, the base and the polymeric acid are introduced separately into the composition, and react on contact to form the water dispersible thickening agent. For example, according to some embodiments, the water thickening agent is TEA-Carbomer® 940.

In addition to TEA-Carbomer® 940, other species which may be used as the water dispersible thickening agent of the mascara composition include magnesium aluminum silicate, TEA-Carbomer® 910, TEA-Carbomer® 934, TEA-Carbomer® 934P, TEA-Carbomer® 941, isopropylamine-Carbomer® 940, sodium hydroxide-Carbomer®940, potassium hydroxide-Carbomer® 940, cellulose gum and xanthan gum. All of which are described in the CTFA Cosmetic Ingredient Dictionary, Third Edition and Third Edition, Supplement, which incorporated herein by reference.

The water dispersible thickening agent can represent between about 0.5% and about 5% by weight of the mascara composition. In some embodiments, the water dispersible thickening agent is present in a concentration in the range of between about 1% and about 4% by weight of the mascara composition. According to other embodiments, the water dispersible thickening agent represents between about 2% and about 3.5% by weight of the mascara composition.

The mascara composition may further comprise a humectant. Suitable humectants include, but are not limited to, glycerin, sorbitol, propylene glycol, glycol, glycol dibehenate, glycol dioctanoate, glycol distearate, glycol hydroxystearate, glycol oleate, glycol ricinoleate, glycol salicylate, glycol stearate, glycol stearate SE, sodium PCA and mixtures thereof. The humectant component of the mascara composition is present is between about 0.1% and about 3% by weight of the composition. According to some embodiments, the humectant comprises between about 0.2% and about 2% by weight of the mascara composition. According to another embodiment, the concentration of humectant represents between about 0.3% and about 1% by weight of the mascara composition.

The mascara composition may further comprise optional components such as a moisturizing agent, a preservative, a sequestering agent and a drying agent. Examples of moisturizing agents include hydrolyzed elastin, hydrolyzed keratin, hydrolyzed silk, hydrolyzed animal protein, hydrolyzed milk protein, hydrolyzed mucopolysaccharides, potassium coco-hydrolyzed animal protein, myristoyl hydrolyzed animal protein and mixtures thereof. In some embodiments, the moisturizing agent is present in a concentration in the range of between about 0.01% and about 1% by weight of the mascara composition.

Examples of preservatives include imidazolidinyl urea, diazolidinyl urea, Quaternium-15, methylparaben, ethylparaben, propylparaben, butylparaben, EDTA and mixtures thereof. In some embodiments, imidazolidinyl urea, methylparaben and mixtures thereof are used in the mascara composition. Preservatives used in the mascara composition may be present in the range of between about 0.01% and about 0.5% by weight.

According to some embodiments, a sequestering agent may be present in a concentration in the range of between about 0.01% and about 0.1% by weight of the mascara composition. In some embodiments, the sequestering agents include disodium EDTA and trisodium EDTA. In some embodiments, the sequestering agent is trisodium EDTA.

Drying agents may be present in the mascara composition in a concentration in the range of between about 1% and less than 5% by weight of the composition. In some embodiments, the drying agents include isopropyl alcohol and at least one alcohol designated as a SD alcohol, which is the designation given by the U.S. Bureau of Alcohol, Tobacco and Firearms to the denaturation method used. For example, SD-40 is ethanol denatured by adding tiny amounts of denatonium benzoate. In some embodiments, at least one of SD Alcohol 1 to SD Alcohol 46 may be used in the mascara composition of this invention.

Generally, water is present in a concentration in the range of between about 60% and about 95%, between about 75% and about 90%, and between about 85% and about 90% by weight of the mascara composition.

In some embodiments, a lecithin-treated cosmetically acceptable pigment, of from about 1.7% to about 4.2% by weight of the mascara composition may be used to produce green, brown, blue and black colored mascaras. In one embodiment wherein a green colored mascara composition is provided by using a mascara composition including between about 3.8% and about 4.2% of chromium hydroxide green by weight of the mascara composition . In another embodiment, brown colored mascara composition is provided by using a mascara composition including between about 0.5% to about 0.7% lecithin-treated iron oxide black by weight of the mascara composition ; between about 0.6% to about 0.8% lecithin-treated iron oxide yellow by weight of the mascara composition; and between about 0.6% to about 0.8% lecithin treated iron oxide red by weight of the mascara composition . In another embodiment, a blue colored mascara is provided by using a mascara composition including between about 2.8% to about 3.2% of lecithin-treated ultramarine blue by weight of the mascara composition . In another embodiment, a black colored mascara composition is provided, by incorporating between about 2.3% and about 2.7% of lecithin-treated iron oxide black by weight of the mascara composition.

In another embodiment, the compositions of the described invention further include one or more additional compatible active ingredients, which are aimed at providing the composition with another pharmaceutical, cosmeceutical or cosmetic effect, in addition to that provided by a compound of the inventive composition.

In one embodiment, the compound of the inventive compositions is an active ingredient.

In another embodiment, the compound of the inventive composition is a new excipient.

Compositions according to the described invention, which further include one or more additional active ingredients, therefore can be further efficiently used, in addition to their use as a treatment for an epithelial-related condition, in the treatment of any medical, cosmetic and/or cosmeceutical condition in which applying the additional active ingredient is beneficial.

Additional active ingredients included in the compositions according to the described invention used to treat an epithelial-related condition selected from the group consisting of sparse hair growth, short hair growth, thin hair growth, alopecia, and hair depigmentation, include, without limitation, one or more, in any combination, of a protective agent, an emollient, an astringent, an irritant, a keratolytic, a sun screening agent, a sun tanning agent, an antibiotic agent, a non-imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent.

Protectives as described herein may take the form of dusting powders, adsorbents, mechanical protective agents, and plasters. Dusting powders are relatively inert and insoluble materials that are used to cover and protect epithelial surfaces, ulcers and wounds. Usually, these substances are finely subdivided powders that absorb moisture and can act as a dessicant. The absorption of skin moisture decreases friction and also discourages certain bacterial growth. Some of the materials used as protective adsorbents include bentonite, insoluble salts of bismuth, boric acid, calcium carbonate, (precipitated), cellulose, corn starch, magnesium stearate, talc, titanium dioxide, zinc oxide, and zinc stearate.

Protectives also can be administered to the skin to form an adherent, continuous film that may be flexible or semi-rigid depending on the materials and the formulations as well as the manner in which they are applied. This material may serve several purposes including providing occlusion from the external environment, providing chemical support, and serving as vehicles for other medicaments. Mechanical protectives are generally either collodions or plasters. Examples include aluminum hydroxide gel, collodium, dimethicone, petrolatum gauze, absorbable gelatin film, absorbable gelatin sponge, zinc gelatin, kaolin, lanolin, anhydrous lanolin, mineral oil, mineral oil emulsion, mineral oil light, olive oil, peanut oil, petrolatum, silicones, hydrocolloids and the like.

In some embodiments, protectives included in the composition of the invention are demulcents.

In some embodiments, the irritant is a rubefacient.

Representative examples of sun screening agents usable in context of the described invention include, without limitation, p-aminobenzoic acid and its salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-propylene glycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzylacetone and benzylacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxyberizophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one) and 4-isopropyl-di-benzoylmethane, and any combination thereof.

Representative examples of sunless tanning agents usable in the described invention include, without limitation, dihydroxyacetone, glyceraldehyde, indoles and their derivatives. The sunless tanning agents can be used in combination with the sunscreen agents.

In some embodiments, the composition further comprises vitaminized peptides. In some embodiments, the composition further comprises vitaminized proteins.

Cleansing agents which may be use in the described invention include surfactant based cleansing agents, examples of which have been listed hereinabove. Other non-surfactant-based cleansing agents known to those of skill in the art may also be employed.

The topical compositions of the described invention can be applied locally to the skin and may be in any form including solutions, oils, creams, ointments, gels, lotions, shampoos, milks, cleansers, moisturizers, sprays, skin patches and the like.

In another embodiment, at least one prostaglandin analog of Formula I, II, or III of the described invention, a carrier and, optionally, additional active ingredients are formed into a composition comprising a solution, emulsion or gel suspension.

In some embodiments, at least one prostaglandin analog of the described invention, a pharmaceutical or cosmetic carrier and, optionally, one or more additional active ingredients are in the form of a solution. A solution can be prepared by mixing a solute or dissolved substance (such as a compound of the invention and, optionally, one or more active ingredient(s)) uniformly throughout a solvent carrier such as water or organic solvents, such as the alcohols (e.g. ethanol or isopropanol, acetone).

Emulsifying agent carriers useful in the described invention are described hereinabove. When the composition of the described invention is an emulsion including a compound of the invention, non-lipid-based vehicles are preferred due to the lipophilic nature of the compounds.

In yet, another embodiment, a composition containing at least one prostaglandin analog of the described invention can be mixed with a gel suspension, (a semi-solid carrier) or solid carrier to form a paste, powder, ointment, cream, lotion, hydrogel or the like.

For example, ointments may be prepared which are in gel-suspension form. These are semi-solid preparations intended for external application to the epithelium. Generally, ointment bases are categorized into hydrocarbon bases (oleaginous), which may use white petroleum as a base; adsorption bases (anhydrous), which might use hydrophilic petroleum or anhydrous lanolin; emulsion bases (water and oil type); emulsion bases (oil and water type); and water soluble bases, which often use polyethylene glycol as an ointment base.

Additional compositions of the described invention can be readily prepared using technology which is known in the art such as described in Remington's Pharmaceutical Sciences, 18th or 19th editions, published by the Mack Publishing Company of Easton, Pa.

In some embodiments, the compositions of the described invention include about 0.001% to about 10.0% w/w of at least one prostaglandin analog of the described invention.

According to another aspect of the described invention, there is provided a method of preparing the compositions described hereinabove. The process generally includes admixing the at least one compound of Formula I, II, or III, at least one compound of Formula IV or Formula V, as described hereinabove, and the pharmaceutically, cosmetically or cosmeceutically acceptable carrier. In cases where additional active ingredients, as detailed above, are present in the compositions, the process includes admixing these ingredients together with the active ingredients and the carrier. The mixing technique utilized in the process of the described invention can involve any one of the known techniques for formulating topical compositions. A variety of exemplary formulation techniques that are usable in the process of the described invention is described, for example, in Harry's Cosmeticology, Seventh Edition, Edited by J B Wilkinson and R J Moore, Longmann Scientific & Technical, 1982.

According to another aspect of the described invention, there is provided a method of treating a medical, cosmetic and/or cosmeceutical condition associated with epithelial tissues that comprises hair loss. The method is effected by topically applying, a pharmaceutically, cosmetically or cosmeceutically effective amount of the composition of the described invention as described above onto an epithelial-related surface.

According to some embodiments of the described invention, the compositions of the described invention are applied topically as needed. According to some embodiments, the inventive compositions are topically applied between one and four times a day, more preferably twice a day (e.g., once in the morning and once in the evening). The topical application of the compositions of the described invention is preferably carried out daily. Some conditions may require topical application for an indeterminate length of time.

In one embodiment, the inventive compositions are topically administered to an epithelial-related surface of a subject. Non limiting examples of epithelial-related surfaces onto which the compositions of the described invention can be applied topically include the lateral aspect of forearms, the lateral aspect of legs, elbows, feet, backhands, back, scalp, face, and any other skin surfaces, and any mucosal membrane described herein.

Alternatively, the compositions may be administered to an epithelial-related condition as a component of, for example, a bandage, adhesive, or transdermal patch. In these instances, the compositions may be an integral component of the bandage, adhesive, or transdermal patch and are thereby applied to the epithelial-related surface.

In some embodiments, the compositions of the described invention are administered to treat an epithelial-related condition that is already present, such as alopecia. In some embodiments, the compositions of the described invention are administered to treat an epithelial-related condition such as, for example, but not limited to, sparse hair growth, short hair growth, thin hair growth, and/or hair de-pigmenation.

The topical therapeutic compositions may be formulated as ophthalmic preparations to treat alopecia of the eyelashes and alopecia of the eyebrows. Ophthalmic preparations for topical administration can include (pharmaceutical) carriers such as sterile and non-sterile aqueous solutions, non-aqueous solutions in common solvents such as alcohols, or solutions comprising at least one compound of Formula I, Formula II, or Formula III in liquid or solid oil bases. Such preparations can be prepared readily using technology known in the art, for example, as described in “The Art, Science and Technology of Pharmaceutical Compounding,” Second Edition, edited by Loyd V. Allen, Jr., Ph.D., and published by the American Pharmaceutical Association of Washington. D.C., and in “Remington's Pharmaceutical Sciences,” 18th or 19th Editions, published by the Mack Publishing Company of Easton, Pa. In preparing an ophthalmic product, a skilled artisan will take into consideration a number of general considerations, including sterility, buffer capacity and pH, tonicity, viscosity, stability, additives, particle size, packaging and preservatives.

Sterile isotonic solutions, properly preserved, are suitable for preparing ophthalmic solutions. In most cases, when the concentration of the active ingredient is low, i.e., less than about 2.5% to about 3.0%, the active ingredient can be dissolved directly in the isotonic vehicle. In some embodiments, compatible excipients, such as preservatives, antioxidants and viscosity enhancers, may be added. For comfort during administration, ophthalmic and nasal solution dosage forms must be “isotonic” with body fluids.

Ophthalmic suspensions are dispersions of finely divided, relatively insoluble ingredients in an aqueous vehicle containing suitable suspending and dispersing agents. Dosage uniformity often requires brisk shaking of the suspension to distribute the suspended substance. The size of particles in an ophthalmic suspension must be micronized so that the particles are small enough to not irritate the eye.

Ophthalmic ointments offer the advantage of longer contact time and greater total bioavailability. The amount of solid released in unit time is a function of concentration, solubility in the ointment base, and diffusivity of the substance in the base. Ophthalmic ointments are prepared so that they do not irritate the eye, do permit diffusion of the active ingredient, and do retain the activity of the active ingredient for a reasonable period of time when stored properly. White petrolatum is the base primarily used for ophthalmic ointments. Powders incorporated in the preparation must be micronized and sterilized to ensure that the final product is nongritty and thus nonirritating.

The described invention described hereinabove has both human and veterinary utility. The term “subject” as used herein includes animals of mammalian origin. Preferably, subjects are human.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Use for Restoring Eyelashes

A composition prepared according to the described invention is formulated as a transparent gel. The formulation comprises water, at least one compound according to Formula I, Formula II, or formula III, according to the described invention (concentration about 0.001% to about 0.07% w/w of the composition), optionally an imidazole analog of Formula IV or Formula V (concentration about 0.001% to about 1.5% w/w of the composition), a thickener, optionally a hair stimulating agent; a chelating agent, at least one penetrating agent, and a preservative, and the pH adjusted to 7.2.

The at least one prostaglandin analog is selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, and 6-(3-chlorophenyl)-17,18,19,20-tetranor PGA₁ N-(1,3-dihydroxypropan-2-yl))amide (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoie acid, (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

The prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB_{I} N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB₁ N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)-N-ethyl-7-(2-((R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof

The prostaglandin C analog is selected from the group consisting of 16-phenoxy-17,18,19,20-tetranor PGG2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, and (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-(1R,2R,58)-5-hydroxy-2-((R)-3-hydroxy-5-phenylpentyl)-3-oxocyclopentyl)hept-5-enoate, (Z)-isopropyl 7-((1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (Z)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, and (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof.

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁ N-(1,3-dihydroxypropan-2-yl))amide, (Z)-isopropyl 7-(1R,2R,3R)-3-hydroxy-2-((R)-3-hydroxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (2)-7-((1R,2R,3R)-3-hydroxy-2-((R,E)-3-hydroxy-4-(3-(trifluoromethyl)phenoxy)but-1-enyl)-5-oxocyclopentyl)hept-5-enoie acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoie acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, and (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 1643-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ_{I} N-(1,3-dihydroxypropan-2-yl))amide; 17-phenyl-18,19,20-trinor PGJ₂ N-cyclopropylamide, 17-phenyl-18,19,20-trinor PGJ_{I} N-cyclopropylmethylamide, 17-phenyl-18,19,20-trinor PGJ₂ N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z)-N-ethyl-7-(1S,5R)-5-((R,E)-3-hydroxy-4-(3-(trifluoromethyl)phenoxy)but-1-enyl)-4-oxocyclopent-2-enyl)hept-5-enamide, (Z)-N-ethyl-7-((1S,5R)-5-((S,E)-3-hydroxy-5-phenylpent-1-enyl)-4-oxocyclopent-2-enyl)hept-5-enamide, (Z)-7-((1S,5R)-5-((R,E)-3-hydroxy-4-(3-(trifluoromethyl)phenoxy)but-1-enyl)-4-oxocyclopent-2-enyl)hept-5-enoic acid, (2)-7-(1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-((1S,5R)-5-((R,E)-4-(3-chlorophenoxy)-3-hydroxybut-1-enyl)-4-oxocyclopent-2-enyl)hept-5-enoic acid, (Z)-isopropyl 7-((1S,5R)-5-((R,E)-4-(3-chlorophenoxy)-3-hydroxybut-1-enyl)-4-oxocyclopent-2-enyl)hept-5-enoate, and (Z)-7-((1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof.

The imidazole analog of Formula IV or Formula V (concentration about 0.001% to about 1.5% w/w of the composition), is added. The imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof.

The gel is applied as a fine line at the skin-eyelash border every night on clean dry eyes. The gel is aqueous enough to spread to the hair follicles but thick enough not to drip.

### Example 2

### Use for Restoring Eyebrows

A composition prepared according to the described invention is formulated as a transparent gel. The formulation comprises water, at least one prostaglandin analog of Formula I, Formula II, or Formula III according to the described invention (concentration about 0.001% to about 0.7% w/w of the composition), optionally an imidazole analog of Formula IV or Formula V (concentration about 0.0001% to about 1.5% w/w of the composition), a thickener, optionally a hair stimulating agent; a chelating agent, at least one penetrating agent, and a preservative, and the pH adjusted to 7.2.

The at least one prostaglandin analog is selected from the group consisting of a prostaglandin A analog, a prostaglandin B analog, a prostaglandin C analog, a prostaglandin D analog, a prostaglandin E analog, a prostaglandin F analog, a prostaglandin 1 analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA_{I} N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid, (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

The prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB_{I} N-(1,3-dihydroxypropan-2-yl))amide; 17-phenyl-18,19,20-trinor PGB₂ N-cyclopropylamide, 17-phenyl-18,19,20-trinor PGB_{I} N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, 6-(3-chlorophenyl)-17,18,19,20-tetranor PGB_{I} N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)-N-ethyl-7-(2-((R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

The prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydr oxy 4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD_{I} N-(1,3-dihydroxypropan-2-yl))amide, (Z)-isopropyl 7-((1R,2R,5S)-5-hydroxy-2-((R)-3-hydroxy-5-phenylpentyl)-3-oxocyclopentyl)hept-5-enoate, (Z)-isopropyl 7-((1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (2)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (Z)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁ N-(1,3-dihydroxypropan-2-yl))amide, (Z)-isopropyl 7-((1R,2R,3R)-3-hydroxy-2-((R)-3-hydroxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-(1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF₂, N-cyclopropylamide, 1643-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGD₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGD₂ N-cyclopropylamide, 17-phenyl-18,19,20-trinor PGJ_{I} N-cyclopropylmethylamide, 17-phenyl-18,19,20-trinor PGJ₂ N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ_{I} N-cyclopropylmethylamide, 6-(3-chlorophenyl)-17,18,19,20-tetranor PGJ₁ N-(1,3-dihydroxypropan-2-yl))amide, (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z) —N-ethyl-7-(1S,5R)-5-((R,E)-3-hydroxy-4-(3-(trifluoromethyl)phenoxy)but-1-enyl)-4-oxocyclopent-2-enyl)hept-5-enamide, (Z)-N-ethyl-7-(1S,5R)-5-((S,E)-3-hydroxy-5-phenylpent-1-enyl)-4-oxocyclopent-2-enyl)hept-5-enamide, (Z)-7-((1S,5R)-5-((R,E)-3-hydroxy-4-(3-(trifluoromethyl)phenoxy)but-1-enyl)-4-oxocyclopent-2-enyl)hept-5-enoic acid, (Z)-7-((1S,5R)-54/2,E)-3-hydroxy-4-(3-(trifluoromethyl)phenoxy)but-1-enyl)-4-oxocyclopent-2-enyl)-N-methylhept-5-enamide, (Z)-7-((1S,5R)-5-((R,E)-4-(3-chlorophenoxy)-3-hydroxybut-1-enyl)-4-oxocyclopent-2-enyl)hept-5-enoic acid, (Z)-isopropyl 7-((1S,5R)-5-((R,E)-4-(3-chlorophenoxy)-3-hydroxybut-1-enyl)-4-oxocyclopent-2-enyl)hept-5-enoate, (Z)-7-((1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof.

The imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof.

The gel is applied by brush as a line at the browbone every night on clean, oil-free brows in the absence of makeup. The gel is aqueous enough to spread to the hair follicles but thick enough not to drip.

### Example 3

### Use for Restoring Scalp Hair

A composition prepared according to the described invention is formulated as an aerosol spray, a topical cream, ointment or a solution.

An aerosol containing approximately 0.005% to about 5.0% (w/w) of at least one prostaglandin analog of Formula I, Formula II, or Formula III according to the described invention, and approximately 0.001% to about 1.5% (w/w) of an imidazole analog, is prepared by dissolving the analog in absolute alcohol. The resulting solution is filtered and chilled to about minus 30 degrees C. A chilled mixture of dichlorodifluoromethane and dichlorotetrafluoroethane is added to the solution. Plastic-coated amber bottles are cold filled with the resulting solution and capped. About 1 cc of the formulation is sprayed on the scalp daily at night.

A topical cream is prepared as follows. Tegacid and spermaceti are melted together at a temperature of about 70 degrees C. to about 80 degree C. Methylparaben is dissolved in water and propylene glycol, polysorbate 80, and at least one prostaglandin analog of Formula I, Formula II, or Formula III (about 0.005% to about 5.0% (w/w)) and optionally an imidazole analog of Formula IV or Formula V (about 0.001% to about 1.5% (w/w)) are added in turn, maintaining the temperature at about 75-80 degree C. The methylparaben mixture is added slowly to the tegacid and spermaceti melt with constant stirring for at least 30 minutes, with additional stirring until the temperature has dropped to 40-45 C. Finally, sufficient water is added to bring the final weight to 10000 gm and the preparation stirred to maintain homogeneity until cooled and congealed. The formulation is applied nightly.

A topical ointment containing at least one prostaglandin analog of Formula I, Formula II, or Formula III (about 0.001% to about 5.0% (w/w)) and optionally an imidazole analog of Formula IV or Formula V (about 0.001% to about 1.5.0% (w/w)) is prepared as follows: White petrolatum and wool fat are melted, strained, and liquid petrolatum added. At least one prostaglandin analog of Formula I, Formula II or Formula III of the described invention and optionally an imidazole analog is added; optionally zinc oxide and calamine may be added as well. The mixture is milled until the powders are finely divided and uniformly dispersed. The mixture is stirred into the white petrolatum, melted and cooled with stirring until the ointment congeals. The formulation is applied nightly.

The at least one prostaglandin analog is selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

The prostaglandin B analog is selected from the group consisting of 16-phenoxy-17,18,19,20-tetranor PGB₂ N-cyclopropylamide, 16-phenoxy-17,18,19,20-tetranor PGB_{I} N-cyclopropylmethylamide, 16-phenoxy-17,18,19,20-PGB₁ N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB_{I} N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)-N-ethyl-7-(2-((R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

The prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl)amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydr oxy-4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((1R,2R,5S)-5-hydroxy-2-((R)-3-hydroxy-5-phenylpentyl)-3-oxocyclopentyl)hept-5-enoate, (Z)-isopropyl 7-((1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (Z)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof.

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁ N-(1,3-dihydroxypropan-2-yl))amide, (Z)-isopropyl 7-((1R,2R,3R)-3-hydroxy-2-((R)-3-hydroxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 1643-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ_{I} N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂ N-(1,3-dihydroxypropan-2-yl)amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z) —N-ethyl-7-(1S,5R)-5-((R,E)-3-hydroxy-4-(3-(trifluoromethyl)phenoxy)but-1-enyl)-4-oxocyclopent-2-enyl)hept-5-enamide, (Z)-N-ethyl-7-(1S,5R)-5-((S,E)-3-hydroxy-5-phenylpent-1-enyl)-4-oxocyclopent-2-enyl)hept-5-enamide, (Z)-7-(1S,5R)-5-((R,E)-3-hydroxy-4-(3-(trifluoromethyl)phenoxy)but-1-enyl)-4-oxocyclopent-2-enyl)hept-5-enoic acid, (Z)-7-(1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-((1S,5R)-5-((R,E)-4-(3-chlorophenoxy)-3-hydroxybut-1-enyl)-4-oxocyclopent-2-enyl)hept-5-enoic acid, (Z)-isopropyl 7-((1S,5R)-5-((R,E)-4-(3-chlorophenoxy)-3-hydroxybut-1-enyl)-4-oxocyclopent-2-enyl)hept-5-enoate, (Z)-7-((1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof.

The imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof.

### Example 4

### Use for Restoring Hair Color

A composition prepared according to the described invention is formulated as an aerosol spray, a topical cream, an ointment or a solution. The total volume used is about 1 cc per application. The formulation is applied nightly. The concentration of the prostaglandin analog of Formula I, Formula II, or Formula III in the composition is about 0.001% to about 5.0% (w/w); the concentration of the optionally added imidazole analog of Formula IV or Formula V is about 0.001% to about 1.5% (w/w).

The at least one prostaglandin analog is selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, and 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid, (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

The prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁ N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, 6-(3-chlorophenyl)-17,18,19,20-tetranor PGB₁ N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)-N-ethyl-7-(2-((1R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

The prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate(Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-(1R,2R,5S)-5-hydroxy-2-((R)-3-hydroxy-5-phenylpentyl)-3-oxocyclopentyl)hept-5-enoate, (Z)-isopropyl 7-((1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,58)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (Z)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof.

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁ N-(1,3-dihydroxypropan-2-yl))amide, (Z)-isopropyl 7-((1R,2R,3R)-3-hydroxy-2-((R)-3-hydroxy-5-phenylpentyl)-5-oxocyclopentyl)hept-5-enoate, (Z)-isopropyl 7-(1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl)amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂ N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z) —N-ethyl-7-(1S,5R)-5-((R,E)-3-hydroxy-4-(3-(trifluoromethyl)phenoxy)but-1-enyl)-4-oxocyclopent-2-enyl)hept-5-enamide, (Z)-N-ethyl-7-((1S,5R)-5-((S,E)-3-hydroxy-5-phenylpent-1-enyl)-4-oxocyclopent-2-enyl)hept-5-enamide, (Z)-7-((1S,5R)-5-((R,E)-3-hydroxy-4-(3-(trifluoromethyl)phenoxy)but-1-enyl)-4-oxocyclopent-2-enyl)hept-5-enoic acid, (Z)-7-((1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-((1S,5R)-5-((R,E)-4-(3-chlorophenoxy)-3-hydroxybut-1-enyl)-4-oxocyclopent-2-enyl)hept-5-enoic acid, (Z)-isopropyl 7-((1S,5R)-5-((R,E)-4-(3-chlorophenoxy)-3-hydroxybut-1-enyl)-4-oxocyclopent-2-enyl)hept-5-enoate, (Z)-7-(1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof.

The imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof.

### Mascara Compositions

Prostaglandin analogs of Formula I, Formula II, or Formula III and optionally added imidazole analogs of Formula IV or Formula V are ingredients of a black colored mascara composition according to Table 1.

TABLE 1
Non-limiting mascara formulations.

| | Black | Brown | Blue | Green |
|---|---|---|---|---|
| | Mascara | Mascara | Mascara | Mascara |
| Component | | | | |
| Water | up to | up to | up to 100 g | up to 100 g |
| | 100 g | 100 g | | |
| SD Alcohol 40-B | 4.5 | 4.5 | 4.5 | 4.5 |
| TEA-Carbomer 940 | 3.275 | 3.3 | 3.3 | 2.94 |
| Polyvinyl alcohol | 1 | 1 | 1 | 1 |
| Glycerin | 0.5 | 0.5 | 0.5 | 0.5 |
| Methylparaben | 0.25 | 0.25 | 0.25 | 0.25 |
| Imidazolidinyl urea | 0.01 | 0.01 | 0.01 | 0.01 |
| Trisodium EDTA | 0.05 | 0.05 | 0.05 | 0.05 |
| Hydrolyzed animal | 0.05 | 0.05 | 0.05 | 0.05 |
| protein | | | | |
| lecithin-treated | | | | |
| pigments | | | | |
| Chromium | | | | 4 |
| hydroxide green | | | | |
| Iron oxide black | 2.5 | 0.6 | | |
| Iron oxide yellow | | 0.7 | | |
| Iron oxide red | | 0.7 | | |
| Ultramarine blue | | | 3 | |
| Prostaglandin | 0.001-5.0 | 0.001-5.0 | 0.001-5.0 | 0.001-5.0 |
| Analog | | | | |
| (Formula I, II, | | | | |
| or III) | | | | |
| Imidazole Analog | 0.001-1.5 | 0.001-1.5 | 0.001-1.5 | 0.001-1.5 |
| (Formula IV or V) | | | | |

### Example 5

### Preparation of a Black Colored Mascara Composition

A black colored mascara composition is prepared by charging about 16.9 parts (by weight) water into a steam jacketed first kettle provided with an agitator. Under agitation 1 part polyvinyl alcohol is added to the water in the first kettle, and mixed for about 15 minutes. An additional charge of water equal to that initially charged, about 16.9 parts, then is added to the agitated mass in the first kettle. After the second charge of water is added, the contents of the first kettle is heated to about 135° F. for about 1 hour under agitation. With no visible solids present, 0.05 part of trisodium EDTA and 0.5 part glycerin is added to the contents of the first kettle. The newly added components are added under agitation for about 15 minutes.

After the contents of the first kettle are visibly completely free of solids, they are transferred to a second steam jacketed, agitator-equipped kettle. The contents during transfer are filtered through a fine mesh filter to insure the absence of solids.

In a separate high shear mixer, equipped with a variable speed propeller, are charged 8 parts water and 1 part of a 2.5% solution of Carbomer 940 in water. These components are agitated while 2.5 parts lecithin treated iron oxide black is added thereto. Agitation is continued until the pigment is completely dispersed. When dispersed, the contents of the high shear mixer are transferred into the second kettle.

A 2.5% solution of Carbomer 940 in water (43 parts) is introduced into a tank provided with an agitator, which is activated during introduction of the 2.5% solution. When the ingredients are smooth and lump-free, they are transferred from the tank to the second kettle. Prior to entering the second kettle, the contents of the tank, are strained through a fine mesh. The combined contents of the second kettle are agitated for about 30 minutes at a temperature of about 135° F. to make the contents smooth and uniform. The contents of the kettle are then cooled to about 90° F. under agitation.

In a premix container equipped with a propeller 1 part water, 0.05 part hydrolyzed animal protein and 0.01 part imidazolidinyl urea are mixed. These ingredients are dissolved into a liquid mass under agitation provided by the propeller and then transferred into the second kettle which is stirred after that addition for about 10 to 15 minutes.

In another premix container 4.5 parts of SD Alcohol 40-B and 0.2 part methylparaben are introduced and mixed to form a clear solution. This solution is added to the contents of the second kettle and mixing of the newly added contents continued for about 30 minutes.

In still another premix container 2.2 parts triethanolamine and 2.2 parts water are mixed until they were clear and uniform. When clear and uniform this mixture is added to the agitated contents of the second kettle. A composition comprising a prostaglandin analog of Formula I, Formula II, or Formula III and an imidazole analog of Formula IV or V then are added to the contents of the second kettle, such that the final concentration of the prostaglandin analog is from about 0.001% to about 5% w/w and the final concentration of the imidazole analog is from about 0.001% to about 1.5% w/w. The contents of the second kettle, the black colored mascara composition, are removed to a storage container.

The at least one prostaglandin analog is selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA₁ N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA₂ N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, and 6-(3-chlorophenyl)-17,18,19,20-tetranor PGA₁ N-(1,3-dihydroxypropan-2-yl))amide; (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid, (2)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

The prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁ N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, 6-(3-chlorophenyl)-17,18,19,20-tetranor PGB₁ N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)-N-ethyl-7-(2-((R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

The prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC_{I} N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydr oxy-4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydr oxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD_{I} N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-(1R,2R,5S)-5-hydroxy-2-((R)-3-hydroxy-5-phenylpentyl)-3-oxocyclopentyl)hept-5-enoate, (Z)-isopropyl 7-(1R,2R,5.5)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (2)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof.

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁ N-(1,3-dihydroxypropan-2-yl))amide; (Z)-isopropyl 7-((1R,2R,3R)-3-hydroxy-2-((R)-3-hydroxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF₂, N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 1643-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGD₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂ N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z) —N-ethyl-7-((1S,5R)-5-((R,E)-3-hydroxy-4-(3-(trifluoromethyl)phenoxy)but-1-enyl)-4-oxocyclopent-2-enyl)hept-5-enamide, (Z)-N-ethyl-7-((1S,5R)-5-((S,E)-3-hydroxy-5-phenylpent-1-enyl)-4-oxacyclopent-2-enyl)hept-5-enamide, (Z)-7-((1S,5R)-5-((R,E)-3-hydroxy-4-(3-(trifluoromethyl)phenoxy)but-1-enyl)-4-oxocyclopent-2-enyl)hept-5-enoic acid, (Z)-7-((1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-((1S,5R)-5-((R,E)-4-(3-chlorophenoxy)-3-hydroxybut-1-enyl)-4-oxocyclopent-2-enyl)hept-5-enoic acid, (Z)-isopropyl 7-((1S,5R)-5-((R,E)-4-(3-chlorophenoxy)-3-hydroxybut-1-enyl)-4-oxocyclopent-2-enyl)hept-5-enoate, (Z)-7-((1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof.

The imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof.

### Example 6

### Preparation of a Brown Colored Mascara Composition

A brown colored mascara composition containing TEA-Carbomer® 940 and a lecithin-treated hydrophobic pigment is prepared in accordance with the procedure of Example 5.

### Example 7

### Preparation of a Blue Colored Mascara Composition

A blue colored mascara composition is prepared in accordance with the procedure of Example 5. The composition differs from that of Example 5 in the identity and concentration of the lecithin-treated pigments and the concentration of the TEA-Carbomer 940 and water components is summarized in the Table.

### Example 8

### Preparation of a Green Colored Mascara Composition

A green colored mascara composition is prepared in accordance with the procedure of Example 5. The composition differs from that of Example 5 only in the concentration of the TEA-Carbomer 940 and water components and the identity and concentration of the lecithin-treated hydrophobic pigments The composition of the green colored mascara composition of this example is summarized in the Table.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A topical composition for treating an epithelial-related condition selected from the group consisting of sparse hair growth, short hair growth, thin hair growth, alopecia, and hair depigmentation of a subject in need thereof, wherein the subject in need thereof is a subject refractory to treatment by a composition comprising a compound of Formula I alone, the composition comprising
(a) a first component and a second component,
(i) the first component comprising:
at least one compound of Formula I or a pharmaceutically acceptable salt, hydrate, solvate, prodrug or metabolite of Formula I, 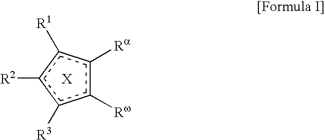
wherein ring X is selected from 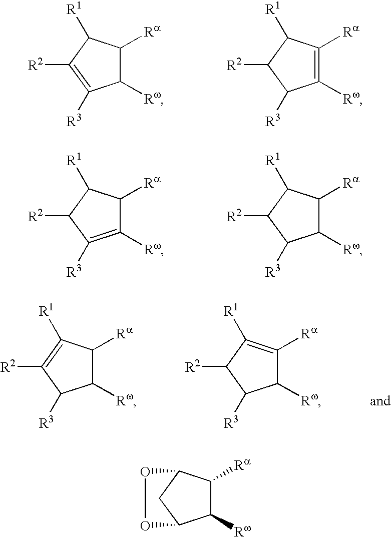
wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each;
wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —PO₂(OH))ₛH where s is 1˜25 or a pharmaceutically acceptable salt thereof; or —P(O)(OH)₂ or a pharmaceutically acceptable salt thereof,
wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁷ groups,
wherein R^{α} is 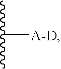
wherein A is a divalent hydrocarbon radical having from two to ten carbon atoms, which may be interrupted by one or more —O— or —S—, zero or one 1,2-phenylene, 1,3-phenylene, or 1,4-phenylene radical, and zero or one 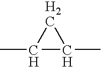
in either cis or trans configuration; the hydrocarbon radical containing zero to four C═C or C≡C bonds and zero to one C═C═C moiety; the hydrocarbon radical having no two heteroatoms adjacent and no heteroatom adjacent to a C—C multiple bond; the hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof,
wherein each R⁷ is independently H, F, or straight chain or branched C₁˜C₅ alkyl
wherein M is C₃˜C₁₀ cycloalkyl containing from one to four rings, C₆˜C₁₀ aryl containing one or two rings or a 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁷ groups
wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃¹; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; —NO₂; —SR¹⁰; —CH═NOR¹⁰; —C(═O)NR¹¹OR¹²; —S(O)₂ NR⁹₂; —NR⁹S(O)₂R¹³; OS(O)²NR⁹₂; —C(O)NHS(O)₂R¹³; —S(O)₂R¹³; —SO₃H; —PO₃H₂; 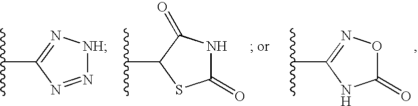
wherein R^{ω} is 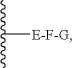
wherein E is a divalent hydrocarbon radical having from two to ten carbon atoms, which may be interrupted by one or more —O— or —S— and zero or one 1,2-phenylene, 1,3-phenylene, or 1,4-phenylene radical; the hydrocarbon radical containing zero to four or C≡C bonds and zero to one C═C═C moiety; the hydrocarbon radical having no two heteroatoms adjacent and no heteroatom adjacent to a non-aromatic C—C multiple bond; the hydrocarbon radical being optionally substituted by one or more —OR⁵, ═O, αS, —O(CO)R⁵ or R⁷ groups; wherein each olefinic moiety may independently be E or Z and each allenic moiety or chiral center may independently possess any relative or absolute stereoconfiguration or any mixture thereof,
wherein F is —CH₂—; —O—; —S—; —S(O)—; —S(O₂); —C(O)—; —C(O)O—; —C(O)S—; —C(O)NR⁹—; —NR⁹—; or a covalent bond,
wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁵ groups,
wherein each R⁸ is independently selected from the group consisting of: 1-1; a pharmaceutically acceptable cation including but not limited to sodium, potassium, magnesium, calcium or an organic cation including but not limited to an ammonium ion; a C₁˜C₂₀ straight chain or branched acyclic hydrocarbon group, which may be interrupted by one or more —O— or —S—, the hydrocarbon group containing zero to four C═C or C≡C bonds wherein each C═C bond independently may be of E or Z configuration, the hydrocarbon group having no two heteroatoms adjacent and no heteroatom adjacent to a non-aromatic C═C or C≡C bond, the hydrocarbon group being substituted with zero to four R'5 groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including but not limited to a lower alkyl ester, a lower acyloxy-alkyl ester (including but not limited to acetoxymethyl, acetoxyethyl, aminocarbonyloxymethyl, pivaloyloxymethyl or pivaloyloxyethyl ester), a lactonyl ester (including but not limited to a phthalidyl or thiophthalidyl ester), a lower alkoxyacyloxyalkyl ester (including but not limited to a methoxycarbonyloxymethyl, ethoxycarbonyloxyethyl or isopropoxycarbonyloxyethyl ester), an alkoxyalkyl ester, choline ester or acylamino alkyl ester (including but not limited to an acetamidomethyl ester); or -J-K, wherein J is a covalent bond or a C₁˜C₁₀ straight chain or branched alkyl and K is C₃˜C₁₀ cycloalkyl containing from one to four rings, C₆—-C₁₀ aryl containing one or two rings or 3˜10 membered heterocycle containing one or two rings and one or more N, O or S atoms, wherein such heterocycle may be aromatic or non-aromatic, the cycloalkyl, aryl or heterocycle being optionally substituted with one to three R¹⁵ groups,
wherein each R⁹ is independently selected from the group consisting of: H; a C₁˜C₂₀ straight chain or branched acyclic hydrocarbon group containing zero to four C═C or C≡C bonds wherein each C═C bond independently may be of E or Z configuration; a C₁˜C₂₀ straight chain or branched acyl group containing zero to four C═C or C≡C bonds wherein each C═C bond independently may be of E or Z configuration; —(CH₂)_{q}OH, —(CH₂)_{q}OR¹⁴, —(CH₂)_{q}OC(O)R₁₄, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁵ groups; —CH₂CH(OH)CH₂OH; —CH(CH₂OH)₂; —CH₂CH(CH₂OH)₂; lower acyloxy-alkyl (including but not limited to acetoxymethyl, acetoxyethyl, aminocarbonyloxymethyl, pivaloyloxymethyl or pivaloyloxyethyl), lactonyl (including but not limited to a phthalidyl or thiophthalidyl), lower alkoxyacyloxyalkyl (including but not limited to a methoxycarbonyloxymethyl, ethoxycarbonyloxyethyl or isopropoxycarbonyloxyethyl), or acylamino alkyl (including but not limited to acetamidomethyl); or -J-K; or —NR⁹₂ may be a cycloamido radical (including but not limited to 1-pyrrolidinyl, 4-morpholinyl, hexahydro-1H-azepin-1-yl, 3-pyrrolin-1-yl, 3,6-dihydro-1(2H)-pyridinyl substituted by one or two R⁹ groups which may be alike or different, or 1-piperazinyl substituted at the 4-position by R⁹, and the like),
wherein each R¹⁰ is independently II; a C₁˜C₂₀ straight chain or branched acyclic hydrocarbon group containing zero to four C═C or C≡C bonds wherein each C═C bond independently may be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO2H, —(CH₂)_{q}OC(O)NH₂, or —(CH₂)_{q}C(O)phenyl, where q is an integer from 1 to 6 inclusive and the phenyl is optionally substituted with one to three R¹⁷ groups; or -L-M, wherein L is a covalent bond or a C₁˜C₁₀ straight chain or branched alkyl,
wherein each R¹¹ is independently H or —C(O)R¹⁶,
wherein each R¹² is independently R¹⁶ or —C(O)R¹⁶,
wherein each R¹³ is independently a C₁˜C₂₀ straight chain or branched acyclic hydrocarbon group, which may be interrupted by one or more —O— or —S—, the hydrocarbon group containing zero to four C═C or C≡C bonds wherein each C═C bond independently may be of E or Z configuration, the hydrocarbon group having no two heteroatoms adjacent and no heteroatom adjacent to a non-aromatic C═C or C═C bond, the hydrocarbon group being substituted with zero to four R¹⁷ groups; —(CH²)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁷ groups; -L-M; or -L-O-M (“O” being oxygen),
wherein each R¹⁴ is independently straight chain or branched C₁˜C₆ alkyl or —CH₂OCH₃,
wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; —CF₃; —C(O)N(R¹⁶)₂; OR10; —N(R¹⁶)C(O)OR¹⁶; —N(R¹⁶)C(O)N(R¹⁶)₂; —OC(O)N(R¹⁶)₂; —N(R¹⁶)C(O)R₅; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(O)NR¹¹OR¹²; —S(O)₂N(R¹⁶)₂; —NR¹⁶S(O)₂R¹³; —C(O)NHS(O)₂R¹³; —S(O)₂R¹³; —SO₃H; —PO₃H₂; 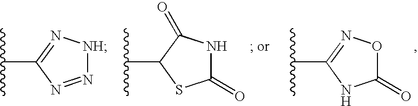
wherein each R¹⁶ is independently H or straight chain or branched C₁˜C₆ alkyl, phenyl or —CH₂OCH₃,
wherein each R¹⁷ is independently straight chain or branched C₁˜C₆ alkyl; —C(O)OR¹⁶; phenyl; F; Cl; Br; I; —CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂ N(R¹⁶)₂; —NR¹⁶S(O)₂R¹³, with the proviso that, if R¹⁷ is —NR¹⁶S(O)₂R¹³, R¹, R², R³, R^{α} and R^{ω} are selected such that the molecular weight of the compound of Formula III does not exceed about 2000 atomic mass units;
(ii) the second component comprising at least one compound of Formula IV or a hydrate, solvate, salt, zwitterion, N-oxide, prodrug, metabolite, or tautomer thereof: 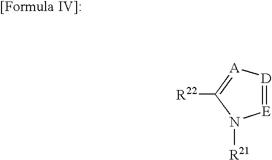
wherein each A is independently N or —NR²⁰;
wherein each D is independently CR²³;
wherein each E is independently N or CR²⁴;
wherein R²³ is H;
wherein R²⁴ is H;
or wherein R²³ and R²⁴ together are —CH═CH—CH═CH—;
wherein R²⁰ is H and the compound of Formula IV is an imidazolium or triazolium salt with a pharmaceutically acceptable counter anion; or
wherein R²⁰ is a moiety that is readily cleaved in vivo, exemplified by, but not limited to, —CH₂OC(O)CH₃, and the compound of Formula IV is a prodrug and an imidazolium or triazolium salt with a pharmaceutically acceptable counter anion;
wherein R²² is H; —CH₃; or 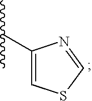
wherein R²¹ is H; —(CR²⁵R²⁶)ₘ—CR²⁷R²⁸-Q-R²⁹;
wherein each m is independently 0 or 1;
wherein R²⁵ is H; —CH₃; or —C≡N;
wherein R²⁶ is H;
wherein R²⁷ is -T-U—V;
wherein R²⁸ is H; OH; or 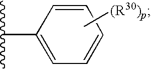
wherein each Q is independently a covalent bond or —S—; wherein R²⁹ is 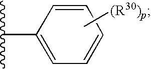
or straight chain or branched C₃˜C₁₀ alkyl optionally substituted with —CO₂H;
wherein each p is independently an integer from 0 to 3 inclusive;
wherein each T is independently a covalent bond; —CH₂CH₂—; —OCH₂—; —CH₂O—; —SCH₂—; —CH₂S—; —OCH₂CH₂O—; —CH(CH₃)—; —CH₂—; or —CF₂—;
wherein each U is independently a covalent bond; 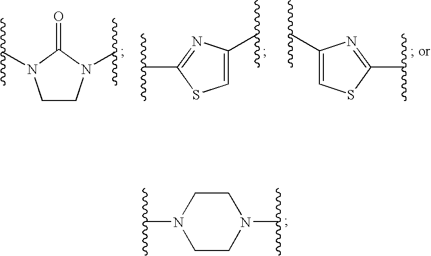
wherein each V is independently H; —S(O)₂CH₃; —C(O)NH₂; —CH₂C≡CH; —CH═CH₂; 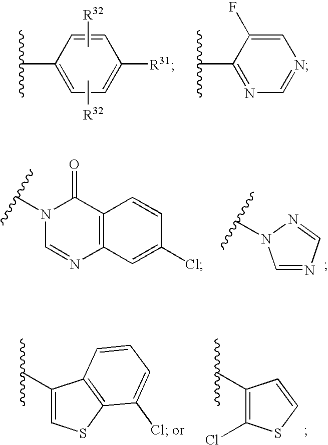
wherein each R³⁰ is independently F, Cl, —CH═CH—CO₂H; 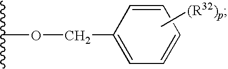
or straight chain or branched C₁˜C₁₀ alkoxy;
wherein each R³¹ is independently H; F; Cl; Br; 1; C≡N; 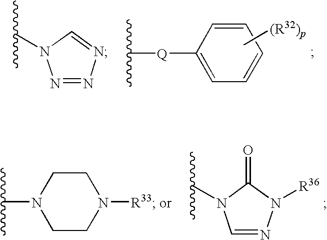
wherein each R³² is independently H; F; Cl; or —OCF₃;
wherein each R³³ is independently straight chain or branched C₁˜C₆ alkyl; —C(O)R³⁴; —C(O)OR³⁴; 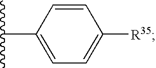
wherein each R³⁴ is independently straight chain or branched C₁˜C₆ alkyl;
wherein each R³⁵ is independently H; 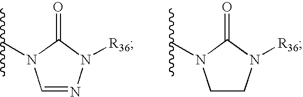
wherein each R³⁶ is independently straight chain or branched C₁˜C₆ alkyl optionally substituted with —OH; or 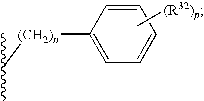
wherein each n is independently 0 or 1;
wherein R²⁶ and R²⁸ together with the two carbon atoms to which they attach may be C═C;
wherein R²⁷ and R²⁸ together may be 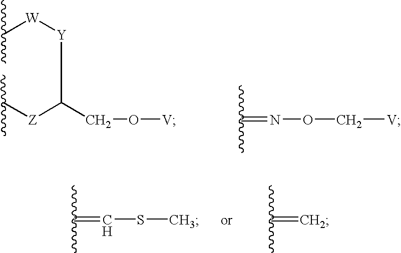
wherein W is —CH₂— and Y is —CH₂—; W is —O— and Y is —CH₂—; or W is —CH₂— and Y is —O—;
wherein Z is —CH₂— or —O—;
wherein R²⁷ together with -Q-R²⁹ may be 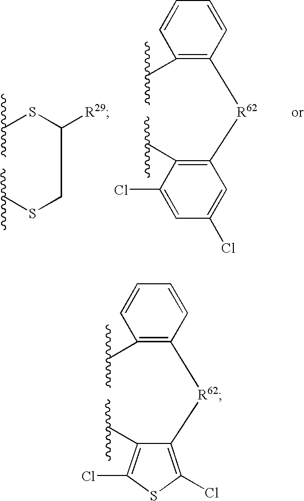
wherein each R⁶² is independently —CH₂CH₂— or —CH═CH—; and
(b) a carrier;
wherein the composition stimulates hair growth on an epithelial surface to which the composition has been applied.

2. The method according to claim 1, wherein the imidazole analog of Formula IV is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a hydrate, solvate, salt, zwitterion, prodrug, metabolite or tautomer thereof.

3. The method according to claim 1, wherein the imidazole analog is miconazole or ketoconazole or a hydrate, solvate, salt, zwitterion, prodrug, metabolite or tautomer thereof.

4. The method according to claim 1, wherein the at least one compound of Formula IV is diastereomerically pure.

5. The method according to claim 1, wherein the at least one compound of Formula IV is a mixture of diastereomers in any ratio.

6. The method according to claim 1, wherein the at least one compound of Formula IV is enantiomerically pure.

7. The method according to claim 1, wherein the at least one compound of Formula IV is a mixture of enantiomers in any ratio, including a racemate.

8. The method according to claim 1, wherein the at least one compound of Formula IV is diastereomerically and enantiomerically pure.

9. The method according to claim 1, wherein the at least one compound of Formula IV is a mixture of diastereomers and enantiomers in any ratio.

10. The method according to claim 1, wherein the at least one compound of Formula IV has one or more hydrogen atoms replaced by deuterium.

11. The method according to claim 1, wherein the at least one compound of Formula I is diastereomerically pure.

12. The method according to claim 1, wherein the at least one compound of Formula I is a mixture of diastereomers in any ratio.

13. The method according to claim 1, wherein the at least one compound of Formula I is enantiomerically pure.

14. The method according to claim 1, wherein the at least one compound of Formula I is a mixture of enantiomers in any ratio, including a racemate.

15. The method according to claim 1, wherein the at least one compound of Formula I is diastereomerically and enantiomerically pure.

16. The method according to claim 1, wherein the at least one compound of Formula I is a mixture of diastereomers and enantiomers in any ratio.

17. The method according to claim 1, wherein the at least one compound of Formula I has one or more hydrogen atoms replaced by deuterium.

18. The method according to claim 1, wherein the at least one compound of Formula I is at least one compound selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

19. The method according to claim 18, wherein the prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 1643-chlorophenyl)-17,18,19,20-tetranor PGA₂ N-cyclopropylamide, 6-(3-chlorophenyl)-17,18,19,20-tetranor PGA_{I} N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoie acid, (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-0,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

20. The method according to claim 18, wherein the prostaglandin B analog is selected from the group consisting of 16-phenoxy-17,18,19,20-tetranor PGB₂ N-cyclopropylamide, 16-phenoxy-17,18,19,20-tetranor PGB_{I} N-cyclopropylmethylamide, 16-phenoxy-17,18,19,20-PGB₁ N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, 6-(3-chlorophenyl)-17,18,19,20-tetranor PGB_{I} N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)-N-ethyl-7-(2-((R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoie acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

21. The method according to claim 18, wherein the prostaglandin C analog is selected from the group consisting of 16-phenoxy-17,18,19,20-tetranor PGC₂ N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

22. The method according to claim 18, wherein the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD_{I} N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD_{I} N-cyclopropylmethylamide, 6-(3-chlorophenyl)-17,18,19,20-tetranor PGD₁ N-(1,3-dihydroxypropan-2-yl))amide, (2)-isopropyl 7-((1R,2R,5S)-5-hydroxy-2-((R)-3-hydroxy-5-phenylpentyl)-3-oxocyclopentyl)hept-5-enoate, (Z)-isopropyl 7-((1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,53)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (Z)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof.

23. The method according to claim 18, wherein the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁ N-(1,3-dihydroxypropan-2-yl))amide, (Z)-isopropyl 7-((1R,2R,3R)-3-hydroxy-2-((R)-3-hydr oxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)bat-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof.

24. The method according to claim 18, wherein the prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

25. The method according to claim 18, wherein the prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ_{I} N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂ N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z)-N-ethyl-7-((1S,5R)-5-((R,E)-3-hydroxy-4-(3-(trifluoromethyl)phenoxy)but-1-enyl)-4-oxocyclopent-2-enyl)hept-5-enamide, (Z)-N-ethyl-7-((1S,5R)-5,E)-3-hydroxy-5-phenylpent-1-enyl)-4-oxocyclopent-2-enyl)hept-5-enamide, (Z)-7-((1S,5R)-5-((R,E)-3-hydroxy-4-(3-(trifluoromethyl)phenoxy)but-1-enyl)-4-oxocyclopent-2-enyl)hept-5-enoic acid, (Z)-7-((1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-((1S,5R)-5-((R,E)-4-(3-chlorophenoxy)-3-hydroxybut-1-enyl)-4-oxocyclopent-2-enyl)hept-5-enoic acid, (2)-isopropyl 7-((1S,5R)-5-((R,E)-4-(3-chlorophenoxy)-3-hydr oxybut-1-enyl)-4-oxocyclopent-2-enyl)hept-5-enoate, (Z)-7-((1S,5R)-5,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof.

26. The topical composition according to claim 1, wherein the topical composition is formulated as a mascara.

27. The topical composition according to claim 1, wherein the topical composition is an ophthalmic composition.

28. The topical composition according to claim 1, wherein the composition restores pigmentation to depigmented hair.

29. The topical composition according to claim 1, wherein the epithelial-related surface onto which the composition is applied topically is an eyelid, at least one eyelash, a face, an eyebrow, a scalp, and above a lip.

30. The topical composition according to claim 1, wherein the composition further comprises at least one additional active ingredient selected from the group consisting of a protective agent, an emollient, an astringent, an irritant, a keratolytic, a sun screening agent, a sun tanning agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent, or a combination thereof.

31. The topical composition according to claim 1, wherein the alopecia is a telogen effluvium type.

32. The topical composition according to claim 31, wherein the telogen effluvium type of alopecia is male pattern baldness.

33. The topical composition according to claim 31, wherein the telogen effluvium type of alopecia is postpartum hair loss.

34. A method for treating an epithelial-related condition selected from the group consisting of sparse hair growth, short hair growth, thin hair growth, alopecia and hair depigmentation, of a subject in need thereof, wherein the subject in need thereof is a subject refractory to treatment by a compound of Formula I alone, the method comprising the steps:
(a) preparing a composition comprising a first component, a second component; and a carrier;
(i) the first component comprising at least one compound of Formula I or a pharmaceutically acceptable salt, hydrate, solvate, prodrug or metabolite thereof, 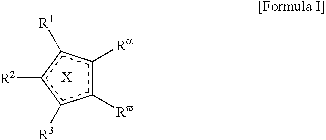
wherein ring X is selected from 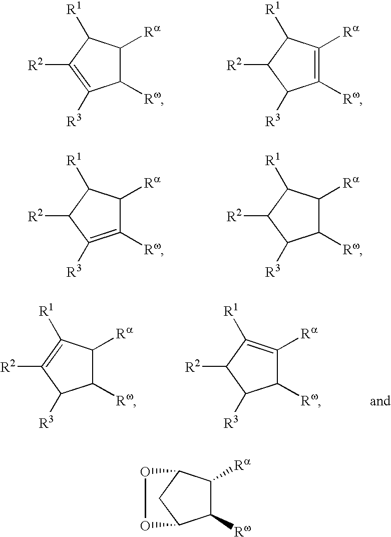
wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each;
wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —PO₂(OH))ₛH where s is 1˜25 or a pharmaceutically acceptable salt thereof or —P(O)(OH)₂ or a pharmaceutically acceptable salt thereof;
wherein each R⁵ is independently H, saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁷ groups;
wherein R^{α} is 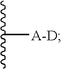
wherein A is a divalent hydrocarbon radical having from two to ten carbon atoms, which may be interrupted by one or more —O— or —S—, zero or one 1,2-phenylene, 1,3-phenylene, or 1,4-phenylene radical, and zero or one 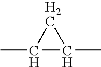
in either cis or trans configuration; the hydrocarbon radical containing zero to four C═C or C≡C bonds and zero to one C═C═C moiety; the hydrocarbon radical having no two heteroatoms adjacent and no heteroatom adjacent to a C—C multiple bond; the hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof;
wherein each R⁷ is independently H, F, or straight chain or branched C₁˜C₅ alkyl;
wherein M is C₃˜C₁₀ cycloalkyl containing from one to four rings, C₆˜C₁₀ aryl containing one or two rings or a 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁷ groups;
wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R'°; —OC(O)R¹⁰; —OR¹⁰; H; —C≡N; —N₃; F; Cl; —CF₃; —CF₂CH₂OH; ; —NO₂; —SR¹⁰; —CH═NOR¹⁰; —C(═O)NR¹¹OR¹²; —S(O)₂ NR⁹₂; —NR⁹S(O)₂R¹³; —OS(O)²NR⁹₂; —C(O)NHS(O)₂R¹³; —S(O)₂R¹³; —SO₃H; —PO₃H₂; 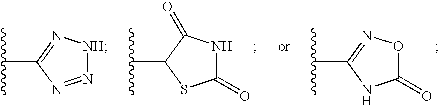
wherein R^{ω} is 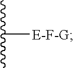
wherein E is a divalent hydrocarbon radical having from two to ten carbon atoms, which may be interrupted by one or more —O— or —S— and zero or one 1,2-phenylene, 1,3-phenylene, or 1,4-phenylene radical; the hydrocarbon radical containing zero to four C═C or C≡C bonds and zero to one C═C═C moiety; the hydrocarbon radical having no two heteroatoms adjacent and no heteroatom adjacent to a non-aromatic C—C multiple bond; the hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof;
wherein F is —CH₂—; —O—; —S—; —S(O)—; —S(O₂)—; —C(O)—; —C(O)O—; —C(O)S—; —C(O)NR⁹—; —NR⁹—; or a covalent bond;
wherein G is H; cycloalkyl; aryl; heterocycle; —CR⁷═N-aryl; —CR⁷═N-heterocycle; wherein cycloalkyl is C₁˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁵ groups;
wherein each R⁸ is independently selected from the group consisting of: H; a pharmaceutically acceptable cation including but not limited to sodium, potassium, magnesium, calcium or an organic cation including but not limited to an ammonium ion; a C₁˜C₂₀ straight chain or branched acyclic hydrocarbon group, which may be interrupted by one or more —O— or —S—, the hydrocarbon group containing zero to four C═C or C≡C bonds wherein each C═C bond independently may be of E or Z configuration, the hydrocarbon group having no two heteroatoms adjacent and no heteroatom adjacent to a non-aromatic C═C or C≡C bond, the hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including but not limited to a lower alkyl ester, a lower acyloxy-alkyl ester (including but not limited to acetoxymethyl, acetoxyethyl, aminocarbonyloxymethyl, pivaloyloxymethyl or pivaloyloxyethyl ester), a lactonyl ester (including but not limited to a phthalidyl or thiophthalidyl ester), a lower alkoxyacyloxyalkyl ester (including but not limited to a methoxycarbonyloxymethyl, ethoxycarbonyloxyethyl or isopropoxycarbonyloxyethyl ester), an alkoxyalkyl ester, choline ester or acylamino alkyl ester (including but not limited to an acetamidomethyl ester); or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle may be aromatic or non-aromatic, the cycloalkyl, aryl or heterocycle being optionally substituted with one to three R¹⁵ groups;
wherein each R⁹ is independently selected from the group consisting of: II; a C₁˜Cₘ straight chain or branched acyclic hydrocarbon group containing zero to four C═C or C≡C bonds wherein each C═C bond independently may be of E or Z configuration; a C₁˜C₂₀ straight chain or branched acyl group containing zero to four C═C or C≡C bonds wherein each C═C bond independently may be of E or Z configuration; —(CH₂)_{q}OH, —(CH₂)_{q}OR¹⁴, —(CH₂)_{q}OC(O)R₁₄, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁵ groups; —CH₂CH(OH)CH₂OH; —CH(CH₂OH)₂; —CH₂CH(CH₂OH)₂; lower acyloxy-alkyl (including but not limited to acetoxymethyl, acetoxyethyl, aminocarbonyloxymethyl, pivaloyloxymethyl or pivaloyloxyethyl), lactonyl (including but not limited to a phthalidyl or thiophthalidyl), lower alkoxyacyloxyalkyl (including but not limited to a methoxycarbonyloxymethyl, ethoxycarbonyloxyethyl or isopropoxycarbonyloxyethyl), or acylamino alkyl (including but not limited to acetamidomethyl); or -J-K; or —NR⁹₂ may be a cycloamido radical (including but not limited to 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which may be alike or different, or 1-piperazinyl substituted at the 4-position by R⁹, and the like);
wherein each R¹⁰ is independently H; a C¹˜C²⁰ straight chain or branched acyclic hydrocarbon group containing zero to four C═C or C≡C bonds wherein each bond independently may be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁷ groups; or -L-M, wherein L is a covalent bond or a C₁˜C₁₀ straight chain or branched alkyl;
wherein each R¹¹ is independently H or —C(O)R¹⁶;
wherein each R¹² is independently R¹⁶ or —C(O)R¹⁶;
wherein each R¹³ is independently a C₁˜C₂₀ straight chain or branched acyclic hydrocarbon group, which may be interrupted by one or more —O— or —S—, the hydrocarbon group containing zero to four C═C or C≡C bonds wherein each C═C bond independently may be of E or Z configuration, the hydrocarbon group having no two heteroatoms adjacent and no heteroatom adjacent to a non-aromatic C═C or C≡C bond, the hydrocarbon group being substituted with zero to four R'⁷ groups; —(CH₂)_{q}OH, —(CH₂)₄OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and the phenyl is optionally substituted with one to three R¹⁷ groups; -L-M; or -L-O-M (“O” being oxygen);
wherein each R¹⁴ is independently straight chain or branched C₁˜C₆ alkyl or —CH₂OCH₃;
wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C1˜C4 alkyl substituted with one, two or three hydroxyl groups; —C(O)OR¹⁶; phenyl; phenyl substituted with one to three R¹⁷; F; Cl; Br; I; —CF₃; —C(O)N(R¹⁶)₂; OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; C(═O)NR¹¹OR¹²; —S(O)₂ N(R¹⁶)₂; —NR¹⁶S(O)₂R¹³; —C(O)NHS(O)₂R¹³; —S(O)₂R¹³; —SO₃H; —PO₃H₂; 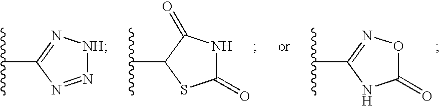
wherein each R¹⁶ is independently H or straight chain or branched C₁˜C₆ alkyl, phenyl or —CH₂OCH₃;
wherein each R¹⁷ is independently straight chain or branched C₁˜C₆ alkyl; —C(O)OR¹⁶; phenyl; F; Cl; Br; I; —CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂N(R¹⁶)₂; —NR¹⁶S(O)₂R¹³, with the proviso that, if R¹⁷ is —NR¹⁶S(O)₂R₁₃, R¹, R², R³, R^{α} and R^{ω} are selected such that the molecular weight of the compound of Formula III does not exceed about 2000 atomic mass units;
(ii) the second component comprising at least one compound of Formula IV or a hydrate, solvate, salt, zwitterion, N-oxide, prodrug, metabolite, or tautomer thereof: 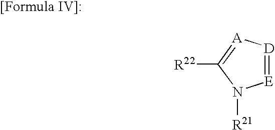
wherein each A is independently N or —NR²⁰;
wherein each D is independently CR²³;
wherein each E is independently N or CR²⁴;
wherein R²³ is H;
wherein R²⁴ is II;
or wherein R²³ and R²⁴ together are —CH═CH—CH═CH—;
wherein R²⁰ is H and the compound of Formula IV is an imidazolium or triazolium salt with a pharmaceutically acceptable counter anion; or
wherein R²⁰ is a moiety that is readily cleaved in vivo, exemplified by, but not limited to, —CH₂OC(O)CH₃, and the compound of Formula IV is a prodrug and an imidazolium or triazolium salt with a pharmaceutically acceptable counter anion; 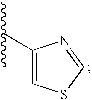
wherein R²² is H; —CH₃; or
wherein R²¹ is H; —(CR²⁵R²⁶)ₘ—CR²⁷R²⁸-Q-R²⁹;
wherein each m is independently 0 or 1;
wherein R²⁵ is H; —CH₃; or —C≡N;
wherein R²⁶ is H;
wherein R²⁷ is -T-U—V;
wherein R²⁸ is H; —OH; or 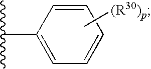
wherein each Q is independently a covalent bond or —S—;
wherein R²⁹ is 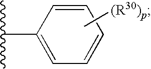
or straight chain or branched C₃˜C₁₀ alkyl optionally substituted with —CO₂H;
wherein each p is independently an integer from 0 to 3 inclusive;
wherein each T is independently a covalent bond; —CH₂CH₂—; —OCH₂—; —CH₂O—; —SCH₂—; —CH₂S—; —OCH₂CH₂O—; —CH(CH₃)—; —CH₂—; or —CF₂—;
wherein each U is independently a covalent bond; 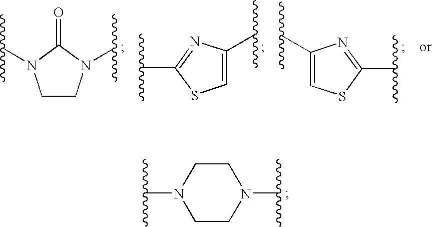
wherein each V is independently H; —S(O)₂CH₃; —C(O)NH₂; —CH₂C═CH; —CH═CH₂; 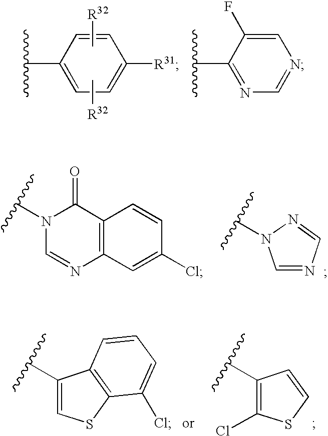
wherein each R³⁰ is independently F, Cl, —CH═CH—CO₂H; 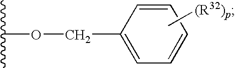
or straight chain or branched C₁˜C₁₀ alkoxy;
wherein each R³¹ is independently H; F; Cl; Br; I; —C≡N; —C≡N 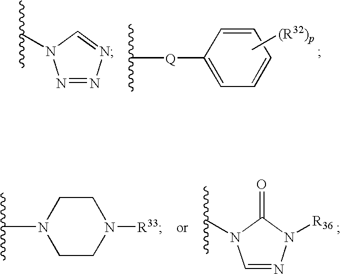
wherein each R³² is independently H; F; Cl; or —OCF₃;
wherein each R³³ is independently straight chain or branched C₁˜C₆ alkyl; —C(O)R³⁴; —C(O)OR³⁴; 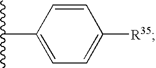
wherein each R³⁴ is independently straight chain or branched C₁˜C₆ alkyl;
wherein each R³⁵ is independently H; 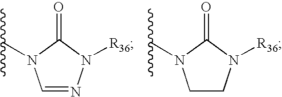
wherein each R³⁶ is independently straight chain or branched C₁˜C₆ alkyl optionally substituted with —OH; or 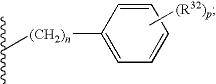
wherein each n is independently 0 or 1;
wherein R²⁶ and R²⁸ together with the two carbon atoms to which they attach may be C═C;
wherein R²⁷ and R²⁸ together may be 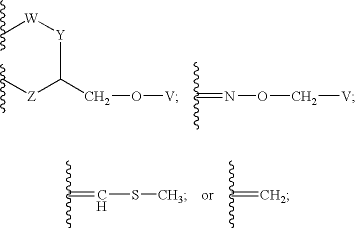
wherein W is —CH₂— and Y is —CH₂—; W is —O— and Y is —CH₂—; or W is —CH₂— and Y is —O—;
wherein Z is —CH₂— or —O—;
wherein R²⁷ together with Q R²⁹ may be 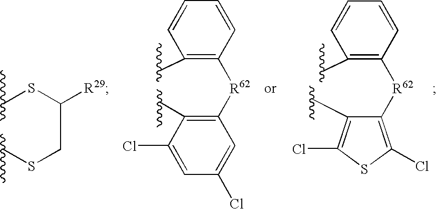
wherein each R⁶² is independently —CH₂CH₂— or —CH═CH—;
wherein each chiral center independently may possess any relative or absolute stereoconfiguration or be any mixture thereof, unless specified otherwise herein;
wherein each olefinic C═C bond that is capable of E/Z isomerism independently may possess either the E or Z stereoconfiguration or be any mixture thereof, unless specified otherwise herein; and
(b) topically applying a cosmetically effective amount of the composition onto an epithelial surface of the subject, and
(c) stimulating hair growth on an epithelial surface to which the composition has been applied.

35. The method according to claim 34, wherein the imidazole analog of Formula IV is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a hydrate, solvate, salt, zwitterion, prodrug, metabolite or tautomer thereof.

36. The method according to claim 34, wherein the imidazole analog is miconazole or ketoconazole or a hydrate, solvate, salt, zwitterion, prodrug, metabolite or tautomer thereof.

37. The method according to claim 34, wherein the at least one compound of Formula IV is diastereomerically pure.

38. The method according to claim 34, wherein the at least one compound of Formula IV is a mixture of diastereomers in any ratio.

39. The method according to claim 34, wherein the at least one compound of Formula IV is enantiomerically pure.

40. The method according to claim 34, wherein the at least one compound of Formula IV is a mixture of enantiomers in any ratio, including a racemate.

41. The method according to claim 34, wherein the at least one compound of Formula IV is diastereomerically and enantiomerically pure.

42. The method according to claim 34, wherein the at least one compound of Formula IV is a mixture of diastereomers and enantiomers in any ratio.

43. The method according to claim 34, wherein the at least one compound of Formula IV has one or more hydrogen atoms replaced by deuterium.

44. The method according to claim 34, wherein the at least one compound of Formula I is diastereomerically pure.

45. The method according to claim 34, wherein the at least one compound of Formula I is a mixture of diastereomers in any ratio.

463. The method according to claim 34, wherein the at least one compound of Formula I is enantiomerically pure.

47. The method according to claim 34, wherein the at least one compound of Formula I is a mixture of enantiomers in any ratio, including a racemate.

48. The method according to claim 34, wherein the at least one compound of Formula I is diastereomerically and enantiomerically pure.

49. The method according to claim 34, wherein the at least one compound of Formula I is a mixture of diastereomers and enantiomers in any ratio.

50. The method according to claim 34, wherein the at least one compound of Formula I has one or more hydrogen atoms replaced by deuterium.

51. The method according to claim 34, wherein the at least one compound of Formula I is at least one compound selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

52. The method according to claim 51, wherein the prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA) N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA₂ N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA) N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid, (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

53. The method according to claim 51, wherein the prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁ N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB_{I} N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)-N-ethyl-7-(2-((R,E)-3-hydroxy-4-(3-(trifluoromethyl)phenoxy)but-1-enyl)-5-oxocyclopent-1-enyl)phept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (2)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, and (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

54. The method according to claim 51, wherein the prostaglandin C analog is selected from the group consisting of 16-phenoxy-17,18,19,20-tetranor PGG2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (2)-isopropyl 7-((R)-2-((R,E)-3-hydroxy-4-(3-(trifluoromethyl)phenoxy)but-1-enyl)-5-oxocyclopent-2-enyl)hept-5-enoate, (Z)-N-ethyl-7-((R)-2-((R,E)-3-hydroxy-4-(3-(trifluoromethyl)phenoxy)but-1-enyl)-5-oxocyclopent-2-enyl)hept-5-enamide, (Z)-N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (2)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

55. The method according to claim 51, wherein the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD_{I} N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (2)-isopropyl 7-((1R,2R,5S)-5-hydroxy-2-((R)-3-hydroxy-5-phenylpentyl)-3-oxocyclopentyl)hept-5-enoate, (Z)-isopropyl 7-(1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (Z)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof.

56. The method according to claim 51, wherein the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁ N-(1,3-dihydroxypropan-2-yl))amide, (Z)-isopropyl 7-((1R,2R,3R)-3-hydroxy-2-((R)-3-hydroxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof.

57. The method according to claim 51, wherein the prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF₂₀, N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

58. The method according to claim 51, wherein the prostaglandin J analog is selected from the group consisting of 16-phenoxy-17,18,19,20-tetranor PGJ₂ N-cyclopropylamide, 16-phenoxy-17,18,19,20-tetranor PGJ_{I} N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂ N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ_{I} N-cyclopropylmethylamide, 6-(3-chlorophenyl)-17,18,19,20-tetranor PGJ₁ N-(1,3-dihydroxypropan-2-yl)amide, (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (Z)-N-ethyl-7-((1S,5R)-5-0,E)-3-hydroxy-4-(3-(trifluoromethyl)phenoxy)but-1-enyl)-4-oxocyclopent-2-enyl)hept-5-enamide, (Z)-N-ethyl-7-((1S,5R)-5-((S,E)-3-hydroxy-5-phenylpent-1-enyl)-4-oxocyclopent-2-enyl)hept-5-enamide, (4-7-((1S,5R)-5-((R,E)-3-hydroxy-4-(3-(trifluoromethyl)phenoxy)but-1-enyl)-4-oxocyclopent-2-enyl)hept-5-enoic acid, (Z)-7-(1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-((1S,5R)-5-((R,E)-4-(3-chlorophenoxy)-3-hydroxybut-1-enyl)-4-oxocyclopent-2-enyl)hept-5-enoic acid, (Z)-isopropyl 7-((1S,5R)-5-((R,E)-4-(3-chlorophenoxy)-3-hydroxybut-1-enyl)-4-oxocyclopent-2-enyl)hept-5-enoate, (Z)-7-((1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof.

59. The method according to claim 1, wherein the topical composition is formulated as a mascara.

60. The method according to claim 34, wherein the topical composition is an ophthalmic composition.

61. The method according to claim 34, wherein the composition restores pigmentation to depigmented hair.

62. The method according to claim 34, wherein the epithelial-related surface onto which the composition is applied topically is an eyelid, at least one eyelash, a face, an eyebrow, a scalp, and above a lip.

63. The method according to claim 34, wherein the composition further comprises at least one additional active ingredient selected from the group consisting of a protective agent, an emollient, an astringent, an irritant, a keratolytic, a sun screening agent, a sun tanning agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent, or a combination thereof.

64. The method according to claim 34, wherein the alopecia is a form of nonscarring alopecia.

65. The method according to claim 63, wherein the nonscarring alopecia is of a telogen effluvium type.

66. The method according to claim 64, wherein the telogen effluvium type of nonscarring alopecia is male pattern baldness.

67. The method according to claim 64, wherein the telogen effluvium type of nonscarring alopecia is postpartum hair loss.

68. A method for treating an epithelial-related condition selected from the group consisting of sparse hair growth, short hair growth, thin hair growth, alopecia and hair depigmentation, the method comprising the steps:
(a) formulating a composition comprising
(i) at least one prostaglandin analog according to Formula IT or a pharmaceutically acceptable salt, hydrate, solvate, prodrug, or metabolite thereof, 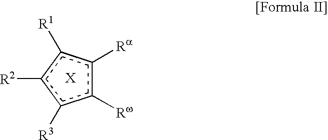
wherein ring X is selected from the group consisting of 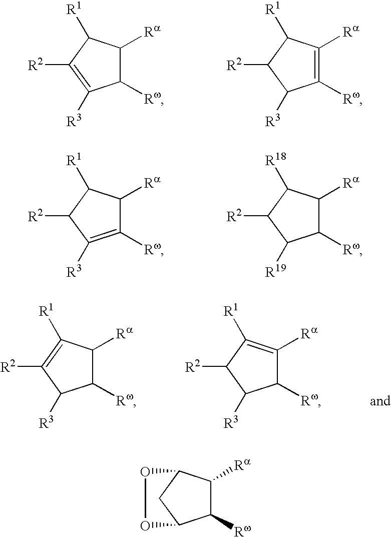
wherein R¹, R², R³, R¹⁸ and R¹⁹ are independently H, —OR⁴, ═O, or —OC(O)R⁵, where the carbon atoms to which R¹, R², R³, R¹⁸ and R¹⁹ attach bear the appropriate number of additional H atoms so as to have exactly 4 bonds each, with the proviso that, when R¹⁸ is —OR⁴ or —OC(O)R⁵ and is cis to R^{α} and trans to R^{ω} with respect to the plane of the cyclopentane ring, then R¹⁹ is H or ═O;
wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; PO₂(OH))ₛH wherein s is 1˜25 or a pharmaceutically acceptable salt thereof; or —P(O)(OH)₂ or a pharmaceutically acceptable salt thereof;
wherein each R⁵ is independently saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups;
wherein R^{α} is 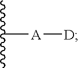
wherein A is a divalent hydrocarbon radical having from two to ten carbon atoms, which can be interrupted by one or more —O— or —S—, zero or one 1,2-phenylene, 1,3-phenylene, or 1,4-phenylene radical, and zero or one 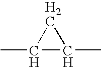
in either cis or trans configuration; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof;
wherein each R⁷ is independently H, F, or straight chain or branched C₁˜C₅ alkyl;
wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups;
wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; —NO₂; —SR¹⁰; —CH═NOR¹⁰; —C(═O)NR¹¹OR¹²; —S(O)₂ NR⁹₂; —NR⁹S(O)₂R¹³; —OS(O)₂ NR⁹₂; —C(O)NHS(O)₂R¹³; —S(O)₂R¹³; —SO₃H; —PO₃H₂; 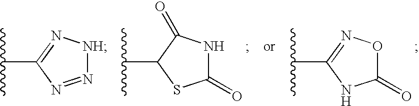
wherein R^{ω} is 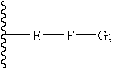
wherein E is a divalent hydrocarbon radical having from two to ten carbon atoms, which can be interrupted by one or more —O— or —S— and zero or one 1,2-phenylene, 1,3-phenylene, or 1,4-phenylene radical; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof;
wherein F is —CH₂—; —S—; —S(O)—; —S(O₂)—; —C(O)—; —C(O)O—; —C(O)S—; —C(O)NR⁹—; —NR⁹—; or a covalent bond;
wherein G is H; cycloalkyl; aryl; heterocycle; —CR^{S}═N-aryl; —CR^{S}═N-heterocycle; wherein cycloalkyl is C₃˜C₁₀ cycloalkyl containing from one to four rings, aryl is C₆—-C₁₀ aryl containing one or two rings, and heterocycle is 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups;
wherein each R⁸ is independently selected from the group consisting of: H; a pharmaceutically acceptable cation including but not limited to sodium, potassium, magnesium, calcium or an organic cation including but not limited to an ammonium ion; a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C═C bond, said hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including but not limited to a lower alkyl ester, a lower acyloxy-alkyl ester (including but not limited to acetoxymethyl, acetoxyethyl, aminocarbonyloxymethyl, pivaloyloxymethyl or pivaloyloxyethyl ester), a lactonyl ester (including but not limited to a phthalidyl or thiophthalidyl ester), a lower alkoxyacyloxyalkyl ester (including but not limited to a methoxycarbonyloxymethyl, ethoxycarbonyloxyethyl or isopropoxycarbonyloxyethyl ester), an alkoxyalkyl ester, choline ester or acylamino alkyl ester (including but not limited to an acetamidomethyl ester); or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups;
wherein each R⁹ is independently selected from the group consisting of: H; a C₁˜C₂₀ straight chain or branched acyclic hydrocarbon group containing zero to four C═C or bonds wherein each C═C bond independently can be of E or Z configuration; a C₁˜C₂₀ straight chain or branched acyl group containing zero to four C═C or bonds wherein each bond independently can be of E or Z configuration; —(CH₂)_{q}OH, —-(CH₂)₄OR¹⁴, —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl (including but not limited to acetoxymethyl, acetoxyethyl, aminocarbonyloxymethyl, pivaloyloxymethyl or pivaloyloxyethyl), lactonyl (including but not limited to a phthalidyl or thiophthalidyl), lower alkoxyacyloxyalkyl (including but not limited to a methoxycarbonyloxymethyl, ethoxycarbonyloxyethyl or isopropoxycarbonyloxyethyl), or acylamino alkyl (including but not limited to acetamidomethyl); or -J-K; or —NR⁹₂ can be a cycloamido radical (including but not limited to 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which can be alike or different, or 1-piperazinyl substituted at the 4-position by R⁹, and the like);
wherein each R¹⁰ is independently H; a C₁˜C₂₀ straight chain or branched acyclic hydrocarbon group containing zero to four C═C or C≡C bonds wherein each C═C bond independently can be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl;
wherein each R¹¹ is independently H or —C(O)R¹⁶;
wherein each R¹² is independently R¹⁶ or —C(O)R¹⁶;
wherein each R¹³ is independently a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C═C bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or -L-O-M (“O” being oxygen);
wherein each R¹⁴ is independently straight chain or branched C₁˜C₆ alkyl or —CH₂OCH₃;
wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three Rⁿ; F; Cl; Br; I; —CF₃; —C(O)N(R¹⁶)₂; —OR¹⁰; —N(R¹⁶)C(O)OR¹⁶; —N(R¹⁶)C(O)N(R¹⁶)₂; —C(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂ N(R¹⁶)₂; —NR¹⁶S(O)₂R¹³; —C(O)NHS(O)₂R¹³; —S(O)₂R¹³; —SO₃H; —PO₃H₂; 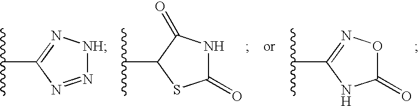
wherein each R¹⁶ is independently H or straight chain or branched C₁˜C₆ alkyl, phenyl or —CH₂OCH₃;
wherein each R¹⁷ is independently straight chain or branched C₁˜C₆ alkyl; —C(O)OR¹⁶; phenyl; F; Cl; Br; I; —CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂ N(R¹⁶)₂; —NR¹⁶S(O)₂R¹³, with the proviso that, if R¹⁷ is —NR¹⁶S(O)₂R¹³, R¹, R², R³, R¹⁸, R¹⁹, R^{α} and R^{ω} are selected such that the molecular weight of the compound of Formula II does not exceed about 2000 atomic mass units; and
wherein not more than four of R⁷ are other than H or F and not more than four of R⁷ are F; and
(ii) a carrier; and
(b) topically applying a cosmetically effective amount of the composition onto an epithelial surface of a subject, including a human, in need thereof.

69. The method according to claim 68, wherein the at least one compound of Formula II is diastereomerically pure.

70. The method according to claim 68, wherein the at least one compound of Formula II is a mixture of diastereomers in any ratio.

71. The method according to claim 68, wherein the at least one compound of Formula II is enantiomerically pure.

72. The method according to claim 68, wherein the at least one compound of Formula II is a mixture of enantiomers in any ratio, including a racemate.

73. The method according to claim 68, wherein the at least one compound of Formula II is diastereomerically and enantiomerically pure.

74. The method according to claim 68, wherein the at least one compound of Formula II is a mixture of diastereomers and enantiomers in any ratio.

75. The method according to claim 68, wherein the at least one compound of Formula II has one or more hydrogen atoms replaced by deuterium.

76. The method according to claim 68, wherein the at least one compound of Formula II is at least one compound selected from the group consisting of a prostaglandin A analog, a prostaglandin B analog, a prostaglandin C analog, a prostaglandin D analog, a prostaglandin E analog, a prostaglandin I analog and a prostaglandin J analog.

77. The method according to claim 76, wherein the prostaglandin A analog is selected from the group consisting of 16-phenoxy-17,18,19,20-tetranor PGA₂ N-cyclopropylamide, 16-phenoxy-17,18,19,20-tetranor PGA_{I} N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, and 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyehept-5-enoate, (Z)-N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)-N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid, (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide, (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

78. The method according to claim 76, wherein the prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB_{I} N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB_{I} N-cyclopropylmethylamide, 6-(3-chlorophenyl)-17,18,19,20-tetranor PGB_{I} N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)-N-ethyl-7-(2-((R,E)-3-hydroxy-4-(3-(trifluoromethyl)phenoxy)but-1-enyl)-5-oxocyclopent-1-enyl)hept-5-enamide, (Z)-N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide
or a pharmaceutically acceptable salt, hydrate, solvate, prodrug or metabolite thereof.

79. The method according to claim 76, wherein the prostaglandin C analog is selected from the group consisting of 16-phenoxy-17,18,19,20-tetranor PGC₂ N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyehept-5-enoate, (Z)-N-ethyl-7-((R)-2-((R,E)-3-hydroxy-4-(3-(trifluoromethyl)phenoxy)but-1-enyl)-5-oxocyclopent-2-enyehept-5-enamide, (Z)-N-ethyl-7-((R)-2-((S,E)-3-hydroxy-S-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyehept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydr oxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydr oxybut-1-enyl)-5-oxocyclopent-2-enyl)-N-methylhept-5-enamide or a pharmaceutically acceptable salt, hydrate, solvate, prodrug or metabolite thereof.

80. The method according to claim 76, wherein the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((1R,2R,5S)-5-hydroxy-2-((R)-3-hydroxy-5-phenylpentyl)-3-oxocyclopentyl)hept-5-enoate, (Z)-isopropyl 7-(1R,2R,5S)-5-hydroxy-2-((R,E)-3-hydroxy-4-(3-(trifluoromethyl)phenoxy)but-1-enyl)-3-oxocyclopentyl)hept-5-enoate, (Z)-N-ethyl-7-((1R,2R,5S)-5-hydroxy-2-((R,E)-3-hydroxy-4-(3-(trifluoromethyl)phenoxy)but-1-enyl)-3-oxocyclopentyl)hept-5-enamide, (Z)-N-ethyl-7-((1R,2R,5S)-5-hydroxy-2-((S,E)-3-hydroxy-5-phenylpent-1-enyl)-3-oxocyclopentyl)hept-5-enamide, (Z)-7-((1R,2R,5S)-5-hydroxy-2-((R,E)-3-hydroxy-4-(3-(trifluoromethyl)phenoxy)but-1-enyl)-3-oxocyclopentyl)hept-5-enoic acid, (Z)-7-((1R,2R,5S)-5-hydroxy-2-((R,E)-3-hydroxy-4-(3-(trifluoromethyl)phenoxy)but-1-enyl)-3-oxocyclopentyl)-N-methylhept-5-enamide, (Z)-7-((1R,2R,5S)-2-((R,E)-4-(3-chlorophenoxy)-3-hydroxybut-1-enyl)-5-hydroxy-3-oxocyclopentyl)hept-5-enoic acid, (Z)-isopropyl 7-((1R,2R,5S)-2-((R,E)-4-(3-chlorophenoxy)-3-hydroxybut-1-enyl)-5-hydroxy-3-oxocyclopentyl)hept-5-enoate, (Z)-7-((1R,2R,5S)-2-((R,E)-4-(3-chlorophenoxy)-3-hydroxybut-1-enyl)-5-hydroxy-3-oxocyclopentyl)-N-methylhept-5-enamide or a pharmaceutically acceptable salt, hydrate, solvate, prodrug or metabolite thereof.

81. The method according to claim 76, wherein the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁ N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁ N-(1,3-dihydroxypropan-2-yl))amide, (Z)-isopropyl 7-((1R,2R,3R)-3-hydroxy-2-((R)-3-hydroxy-5-phenylpentyl)-5-oxocyclopentyl)hept-5-enoate, (Z)-isopropyl 7-((1R,2R,3R)-3-hydroxy-2-((R,E)-3-hydroxy-4-(3-(trifluoromethyl)phenoxy)but-1-enyl)-5-oxocyclopentyl)hept-5-enoate, (Z)-N-ethyl-7-((1R,2R,3R)-3-hydroxy-2-((R,E)-3-hydroxy-4-(3-(trifluoromethyl)phenoxy)but-1-enyl)-5-oxocyclopentyl)hept-5-enamide, (Z)-N-ethyl-7-((1R,2R,3R)-3-hydroxy-2-((S,E)-3-hydroxy-5-phenylpent-1-enyl)-5-oxocyclopentyl)hept-5-enamide, (Z)-7-((1R,2R,3R)-3-hydroxy-2-((R,E)-3-hydroxy-4-(3-(trifluoromethyl)phenoxy)but-1-enyl)-5-oxocyclopentyl)hept-5-enoic acid, (Z)-7-((1R,2R,3R)-3-hydroxy-2-((R,E)-3-hydroxy-4-(3-(trifluoromethyl)phenoxy)but-1-enyl)-5-oxocyclopentyl)-N-methylhept-5-enamide, (Z)-7-((1R,2R,3R)-2-((R,E)-4-(3-chlorophenoxy)-3-hydroxybut-1-enyl)-3-hydroxy-5-oxocyclopentyl)hept-5-enoic acid, (Z)-isopropyl 7-((1R,2R,3R)-2-((R,E)-4-(3-chlorophenoxy)-3-hydroxybut-1-enyl)-3-hydroxy-5-oxocyclopentyl)hept-5-enoate, (Z)-7-((1R,2R,3R)-2-((R,E)-4-(3-chlorophenoxy)-3-hydroxybut-1-enyl)-3-hydroxy-5-oxocyclopentyl)-N-methylhept-5-enamide or a pharmaceutically acceptable salt, hydrate, solvate, prodrug or metabolite thereof.

82. The method according to claim 76, wherein the prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ_{I} N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGD₂ N-(1,3-dihydroxypropan-2-yl)amide; 16-(3-chlorophenyl)-17,18,19,20-tetranor PGJ₂ N-cyclopropylamide, 16-(3-chlorophenyl)-17,18,19,20-tetranor PGJ_{Z} N-cyclopropylmethylamide, 6-(3-chlorophenyl)-17,18,19,20-tetranor PGJ_{I} N-(1,3-dihydroxypropan-2-yl))amide, (Z)-isopropyl 7-((1S,5R)-5-((R)-3-hydroxy-5-phenylpentyl)-4-oxocyclopent-2-enyl)hept-5-enoate, (Z)-isopropyl 7-((1S,5R)-5-((R,E)-3-hydroxy-4-(3-(trifluoromethyl)phenoxy)but-1-enyl)-4-oxocyclopent-2-enyl)hept-5-enoate, (2) —N-ethyl-7-((1S,5R)-5-((R,E)-3-hydroxy-4-(3-(trifluoromethyl)phenoxy)but-1-enyl)-4-oxocyclopent-2-enyl)hept-5-enamide, (Z)-N-ethyl-7-((1S,5R)-5-((S,E)-3-hydroxy-5-phenylpent-1-enyl)-4-oxocyclopent-2-enyl)hept-5-enamide, (Z)-7-((1S,5R)-5-((R,E)-3-hydroxy-4-(3-(trifluoro34-methyl)phenoxy)but-1-enyl)-4-oxocyclopent-2-enyl)hept-5-enoic acid, (Z)-7-(1S,5R)-5-((R,E)-3-hydroxy-4-(3-(trifluoromethyl)phenoxy)but-1-enyl)-4-oxocyclopent-2-enyl)-N-methylhept-5-enamide, (Z)-7-((1S,5R)-5-((R,E)-4-(3-chlorophenoxy)-3-hydroxybut-1-enyl)-4-oxocyclopent-2-enyl)hept-5-enoic acid, (Z)-isopropyl 7-((1S,5R)-5-((R,E)-4-(3-chlorophenoxy)-3-hydroxybut-1-enyl)-4-oxocyclopent-2-enyl)hept-5-enoate, (Z)-7-(1S,5R)-5-((R,E)-4-(3-chlorophenoxy)-3-hydroxybut-1-enyl)-4-oxocyclopent-2-enyl)-N-methylhept-5-enamide or a pharmaceutically acceptable salt, hydrate, solvate, prodrug or metabolite thereof.

83. The method according to claim 68, wherein the topical composition is formulated as a mascara.

84. The method according to claim 68, wherein the topical composition is an ophthalmic composition.

85. The method according to claim 68, wherein the composition restores pigmentation to depigmented hair.

86. The method according to claim 68, wherein the epithelial-related surface onto which the composition is applied topically is an eyelid, at least one eyelash, a face, an eyebrow, a scalp, and above a lip.

87. The method according to claim 68, wherein the composition further comprises at least one additional active ingredient selected from the group consisting of a protective agent, an emollient, an astringent, an irritant, a keratolytic, a sun screening agent, a sun tanning agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent, or a combination thereof.

88. The method according to claim 68, wherein the alopecia is a form of nonscarring alopecia.

89. The method according to claim 88, wherein the nonscarring alopecia is of a telogen effluvium type.

90. The method according to claim 89, wherein the telogen effluvium type of nonscarring alopecia is male pattern baldness.

91. The method according to claim 89, wherein the telogen effluvium type of nonscarring alopecia is postpartum hair loss.
